(19) United States
(12) Patent Application Publication
(10) Pub. No.: US 2009/0004281 A1
(43) Pub. Date: Jan. 1, 2009

(54) MULTIPARTICULATE OSMOTIC DELIVERY SYSTEM

(75) Inventors: Tien Nghiem, Dublin (IE); Graham JACKSON, Co. Kildare (IE)

Correspondence Address: OBLON, SPIVAK, MCCLELLAND MAIER & NEUSTADT, P.C., 1940 DUKE STREET, ALEXANDRIA, VA, 22314, US

(73) Assignee: BIOVAIL LABORATORIES INTERNATIONAL S.R.L., St. Michael (BB)

(21) Appl. No.: 11/768,764

(22) Filed: Jun. 26, 2007

(51) Int. Cl. A61K 9/50; A61K 31/27; A61K 31/428; A61P 25/16; A61P 9/06; A61P 25/28; A61P 25/00; A61K 31/554

(52) U.S. Cl. 424/490; 514/211.07; 514/367; 514/490

(57) ABSTRACT

The present invention relates to a multiparticulate osmotic delivery system. The system is a modified release composition suitable for oral administration. The composition includes a core that includes at least one drug in combination with at least one pharmaceutically acceptable excipient. The composition further includes an osmotic subcoat surrounding the core, and a modified release overcoat surrounding the osmotic subcoated core.

The present invention relates to modified release compositions for, preferably, oral administration of a drug. In particular, the present invention relates to a multiparticulate osmotic delivery system for a modified release of at least one drug.

A preferred dosage regimen for many medications is that by which a therapeutically effective concentration of drug at the site(s) of action is attained and maintained constant for the duration of the treatment. Providing dose size and frequency of administration are correct, therapeutic “steady-state” plasma concentrations of a drug can be achieved and maintained by the repetitive administration of conventional peroral dosage forms. However, there are a number of potential limitations associated with conventional peroral dosage forms. These limitations have led pharmaceutical scientists to consider presenting therapeutically active molecules in “modified release”, e.g. “extended-release” or “controlled-release”, formulations.

Oral ingestion is the traditionally preferred route of drug administration, providing a convenient method of effectively achieving both local and systemic effects. A preferred oral drug delivery system should steadily deliver a measurable and reproducible amount of drug to the target site over a prolonged period. Extended-release (ER) or controlled-release (CR) delivery systems provide a uniform concentration/amount of the drug at the absorption site and thus, after absorption, allow maintenance of plasma concentrations within a therapeutic range over an extended period of time, which can minimize side effects and reduce the frequency of administration. ER or CR dosage forms release drug slowly, so that plasma concentrations are maintained at a therapeutic level for a prolonged period of time. These products can provide benefits compared with immediate-release compositions, such as greater effectiveness in the treatment of chronic conditions, reduced side effects, greater convenience, and/or higher levels of patient compliance due to a simplified dosing schedule. Because of the above advantages, such systems form a major segment of the drug delivery market.

Over the years many drug delivery systems have been developed with the aim of eliminating the cyclical changes in plasma drug concentration seen after the administration of a conventional delivery system.

Generally, modified release pharmaceutical compositions can be characterized as either diffusion controlled delivery systems or osmotic dispensing devices. The conventional osmotic device is based on an internal/external osmotic pressure differential (e.g., osmotic pressure gradient across a water-permeable wall against an external fluid) for controlling drug release. Early osmotic devices were in the form of tablets that included a core composition of drug and an osmotic agent enclosed by an insoluble water-permeable membrane having a release means. In such osmotic devices, aqueous body fluids could enter the osmotic system continuously through the water-permeable membrane and dissolve the solid drug contained within the core. The drug can then be released through an orifice in the membrane, once sufficient pressure is built up, to cause the solution containing the drug to be pushed through an orifice. When the drug present in the core is able to produce a sufficiently high osmotic pressure of its own or when additives are present to increase the osmotic pressure (e.g., osmotic agents), the drug can then be released to achieve the desired therapeutic effect. In these early osmotic devices, the prerequisite for achieving the desired therapeutic effect was a sufficiently high solubility of water-soluble drug such that the amount of water entering the core through the water-permeable membrane was sufficient to dissolve most of the drug in the core. As a result, the drug would be delivered from the tablet in a predominantly soluble form. For drugs that are insoluble or have low-solubility in the fluid environment (e.g., bodily fluids), osmotically controlled delivery of the drug to elicit the desired therapeutic effect would be more difficult with such early osmotic devices. Numerous modifications to the early types of osmotic devices have been described in the art in an effort to improve their release characteristics.

Modified release dosage forms utilizing both diffusion and osmotic mechanisms to control drug release have also been described in the art. Dosage forms in this category include tablets with a mixture of drug and an osmotically effective solute. The tablets are covered with either a rate controlling water permeable coat or a water impermeable coat with an aperture. A disadvantage to this type of system is that the total amount of drug is contained in a single unit, which can lead to complications such as dose dumping.

In contrast to the single unit dosage form, prior art multiparticulate systems have been developed which contain multiple particles each containing a small fraction of the drug. Advantages of multiparticulate systems over single unit dosage forms can include increased bioavailability, reduced risk of systemic toxicity due to dose dumping, reduced risk of local irritation, and reduced variation in gastric transit time. Certain multiparticulate osmotic systems are known to comprise particles that include a core made of the drug mixed with an osmotic agent and other excipients, and a rate-controlling coat surrounding the core. Such multiparticulate systems can include limitations that result from the mixing of the drug with the osmotic agent within the core. Such mixing can lead to unfavorable interactions that can decrease the stability of the osmotic system. For example, in certain known systems the osmotic agent attracts water, which can lead to degradation of the drug because of its close proximity to the hygroscopic osmotic agent. Known systems that use sugar spheres in the core have a limitation in that a sealcoat is required to control i.e., reduce or eliminate) the osmotic effect of the sucrose in the core. Other limitations of known multiparticulate systems relate to the rate-controlling coat that surrounds the core. For example, the rate-controlling coat of certain prior art systems require pore-forming additives in order to provide the desired drug release. The presence of pore-forming additives in the rate-controlling coat can make it difficult to predict the drug release profile of the coated particles. The location, size and density of the pores can be difficult to predict, and can have a direct effect on the drug release profile. A further limitation of many prior art systems is incomplete drug release. This is especially the case with prior art formulations of low solubility drugs and/or low-dose drugs. Other limitations of prior art modified release multiparticulate osmotic systems include; restriction to a single type of modified drug release (e.g. delayed-release, or sustained-release compositions but not both); and difficulty in controlling the osmotic effect.

As such, there remains a need in the art for a stable, more easily controllable multiparticulate osmotic system that can provide increased release of the drug(s) and that can be used in a variety of modified-release compositions and in a variety of dosage forms.

According to an aspect of the present invention, there is provided a modified release pharmaceutical composition for oral administration suitable for once daily dosing comprising: (i) a core including at least one drug; (ii) an osmotic subcoat including at least one osmotic agent and at least one osmotic deposition vehicle, wherein the osmotic subcoat at least partially or fully surrounds the core; and (iii) a modified release coating at least partially or fully surrounding the osmotic subcoat. Thus, the modified release coating could surround from about 1% to about 100% of the core, including about 1%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% of the core, including all values, ranges and subranges therebetween, thus including, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, or from about 95% to about 100% of the osmotic subcoat. Similarly, the osmotic subcoat could surround from about 1% to about 100%, including about 1%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% of the core, including all values, ranges and subranges therebetween, thus including, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, or from about 95% to about 100% of the core. The composition provides modified release and increased release of the at least one drug.

In at least one implementation of the first aspect, the composition provides increased release of the drug(s) and exhibits a dissolution profile such that substantially full release of the drug(s) can be achieved in from about 3 hours to about 24 hours, including about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, including all values, ranges and subranges therebetween, thus including, for example, from about 4 to about 24 hours, from about 5 to about 24 hours, from about 6 to about 24 hours, from about 7 to about 24 hours, from about 8 to about 24 hours, from about 9 to about 24 hours, from about 10 to about 24 hours, from about 11 to about 24 hours, from about 12 to about 24 hours, from about 13 to about 24 hours, from about 14 to about 24 hours, from about 15 to about 24 hours, from about 16 to about 24 hours, from about 17 to about 24 hours, from about 18 to about 24 hours, from about 19 to about 24 hours, from about 20 to about 24 hours, from about 21 to about 24 hours, from about 22 to about 24 hours, or from about 23 to about 24 hours. In at least one implementation of the first aspect, the composition exhibits a dissolution profile such that substantially full release of the drug(s) is achieved in about 24 hours.

In at least one implementation of the first aspect, the composition provides an in vitro dissolution profile such that after about 2 hours from about 0% to about 40% by weight of the drug(s) is released, including all values, ranges and subranges therebetween, thus including, for example, about 5% by weight of the drug(s) is released, about 10% by weight of the drug(s) is released, about 15% by weight of the drug(s) is released, about 20% by weight of the drug(s) is released, about 25% by weight of the drug(s) is released, about 30% by weight of the drug(s) is released, or about 35% by weight of the drug(s) is released; after about 4 hours from about 0% to about 60% by weight of the drug(s) is released, including all values, ranges and subranges therebetween, thus including, for example, about 5% by weight of the drug(s) is released, about 10% by weight of the drug(s) is released, about 15% by weight of the drugs(s) is released, about 20% by weight of the drug(s) is released, about 25% by weight of the drug(s) is released, about 30% by weight of the drug(s) is released, about 35% by weight of the drug(s) is released, about 40% by weight of the drug(s) is released, about 45% by weight of the drug(s) is released, about 50% by weight of the drug(s) is released, or about 55% by weight of the drug(s) is released; after about 6 hours from about 0% to about 80% by weight of the drug(s) is released, including all values, ranges and subranges therebetween, thus including, for example, about 5% by weight of the drug(s) is released, about 10% by weight of the drug(s) is released, about 15% by weight of the drugs(s) is released, about 20% by weight of the drug(s) is released, about 25% by weight of the drug(s) is released, about 30% by weight of the drug(s) is released, about 35% by weight of the drug(s) is released, about 40% by weight of the drug(s) is released, about 45% by weight of the drug(s) is released, about 50% by weight of the drug(s) is released, about 55% by weight of the drug(s) is released, about 60% by weight of the drug(s) is released, about 65% by weight of the drug(s) is released, about 70% by weight of the drug(s) is released, or about 75% by weight of the drug(s) is released; after about 8 hours from about 0% to about 90% by weight of the drug(s) is released, including all values, ranges and subranges therebetween, thus including, for example, about 5% by weight of the drug(s) is released, about 10% by weight of the drug(s) is released, about 15% by weight of the drugs(s) is released, about 20% by weight of the drug(s) is released, about 25% by weight of the drug(s) is released, about 30% by weight of the drug(s) is released, about 35% by weight of the drug(s) is released, about 40% by weight of the drug(s) is released, about 45% by weight of the drug(s) is released, about 50% by weight of the drug(s) is released, about 55% by weight of the drug(s) is released, about 60% by weight of the drug(s) is released, about 65% by weight of the drug(s) is released, about 70% by weight of the drug(s) is released, about 75% by weight of the drug(s) is released, about 80% by weight of the drug(s) is released, or about 85% by weight of the drug(s) is released; and after about 12 hours from about 0% to about 100% by weight of the drug(s) is released, including all values, ranges and subranges therebetween, thus including, for example, about 5% by weight of the drug(s) is released, about 10% by weight of the drug(s) is released, about 15% by weight of the drugs(s) is released, about 20% by weight of the drug(s) is released, about 25% by weight of the drug(s) is released, about 30% by weight of the drug(s) is released, about 35% by weight of the drug(s) is released, about 40% by weight of the drug(s) is released, about 45% by weight of the drug(s) is released, about 50% by weight of the drug(s) is released, about 55% by weight of the drug(s) is released, about 60% by weight of the drug(s) is released, about 65% by weight of the drug(s) is released, about 70% by weight of the drug(s) is released, about 75% by weight of the drug(s) is released, about 80% by weight of the drug(s) is released, about 85% by weight of the drug(s) is released, about 90% by weight of the drug(s) is released, or about 95% by weight of the drug(s) is released.

In at least one implementation of the first aspect, the composition provides an in vitro dissolution profile using a USP II paddle apparatus, pH 6.8 phosphate buffer, and a rotation of 50 rpm, such that after about 2 hours from about 0% to about 8% by weight of the drug(s) is released, including all values, ranges and subranges therebetween, thus including, for example, about 1% by weight of the drug(s) is released, or about 2% by weight of the drug(s) is released, or about 3% by weight of the drug(s) is released, or about 4% by weight of the drug(s) is released, or about 5% by weight of the drug(s) is released, or about 6% by weight of the drug(s) is released, or about 7% by weight of the drug(s) is released; after about 4 hours from about 0% to about 24% by weight of the drug(s) is released, including all values, ranges and subranges therebetween, thus including, for example, about 1% by weight of the drug(s) is released, or about 2% by weight of the drug(s) is released, or about 3% by weight of the drug(s) is released, or about 4% by weight of the drug(s) is released, or about 5% by weight of the drug(s) is released, or about 6% by weight of the drug(s) is released, or about 7% by weight of the drug(s) is released, or about 8% by weight of the drug(s) is released, or about 9% by weight of the drug(s) is released, or about 10% by weight of the drug(s) is released, or about 11% by weight of the drug(s) is released, or about 12% by weight of the drug(s) is released, or about 13% by weight of the drug(s) is released, or about 14% by weight of the drug(s) is released, or about 15% by weight of the drugs(s) is released, or about 16% by weight of the drug(s) is released, or about 17% by weight of the drug(s) is released, or about 18% by weight of the drug(s) is released, or about 19% by weight of the drug(s) is released, or about 20% by weight of the drug(s) is released, or about 21% by weight of the drug(s) is released, or about 22% by weight of the drug(s) is released, or about 23% by weight of the drug(s) is released; after about 6 hours from about 2% to about 47% by weight of the drug(s) is released, including all values, ranges and subranges therebetween, thus including, for example, about 3% by weight of the drug(s) is released, or about 4% by weight of the drug(s) is released, or about 5% by weight of the drug(s) is released, or about 6% by weight of the drug(s) is released, or about 7% by weight of the drug(s) is released, or about 8% by weight of the drug(s) is released, or about 9% by weight of the drug(s) is released, or about 10% by weight of the drug(s) is released, or about 11% by weight of the drug(s) is released, or about 12% by weight of the drug(s) is released, or about 13% by weight of the drug(s) is released, or about 14% by weight of the drug(s) is released, or about 15% by weight of the drugs(s) is released, or about 16% by weight of the drug(s) is released, or about 17% by weight of the drug(s) is released, or about 18% by weight of the drug(s) is released, or about 19% by weight of the drug(s) is released, or about 20% by weight of the drug(s) is released, or about 21% by weight of the drug(s) is released, or about 22% by weight of the drug(s) is released, or about 23% by weight of the drug(s) is released, or about 24% by weight of the drug(s) is released, or about 25% by weight of the drug(s) is released, or about 26% by weight of the drug(s) is released, or about 27% by weight of the drug(s) is released, or about 28% by weight of the drug(s) is released, or about 29% by weight of the drug(s) is released, or about 30% by weight of the drug(s) is released, or about 31% by weight of the drug(s) is released, or about 32% by weight of the drug(s) is released, or about 33% by weight of the drug(s) is released, or about 34% by weight of the drug(s) is released, or about 35% by weight of the drug(s) is released, or about 36% by weight of the drug(s) is released, or about 37% by weight of the drug(s) is released, or about 38% by weight of the drug(s) is released, or about 39% by weight of the drug(s) is released, or about 40% by weight of the drug(s) is released, or about 41% by weight of the drug(s) is released, or about 42% by weight of the drug(s) is released, or about 43% by weight of the drug(s) is released, or about 44% by weight of the drug(s) is released, or about 45% by weight of the drug(s) is released, or about 46% by weight of the drug(s) is released; after about 8 hours from about 2% to about 67% by weight of the drug(s) is released, thus including all values, ranges, and subranges therebetween, thus including, for example, from about 10% to about 67% by weight of the drug(s) is released, or from about 20% to about 67% by weight of the drug is released, or from about 30% to about 67% by weight of the drug(s) is released, or from about 40% to about 67% by weight of the drug(s) is released, or from about 50% to about 67% by weight of the drug(s) is released, or about 51% by weight, about 52% by weight, about 53% by weight, about 54% by weight, about 55% by weight, about 56% by weight, about 57% by weight, about 58% by weight, about 59% by weight, about 60% by weight, about 61% by weight, about 62% by weight, about 63% by weight, about 64% by weight, about 65% by weight, about 66% by weight, or about 67% by weight of the drug(s) is released; after about 12 hours from about 29% to about 86% by weight of the drug(s) is released, thus including all values, ranges and subranges therebetween, for example, from about 30% to about 86% by weight of the drug(s) is released, or from about 40% to about 86% by weight of the drug(s) is released, or from about 50% to about 86% by weight of the drug(s) is released, or from about 60% to about 86% by weight of the drug(s) is released, or from about 70% to about 86% of the drug(s) is released, or from about 80% to about 86% by weight of the drug(s) is released, or about 81% by weight, or about 82% by weight, or about 83% by weight, or about 84% by weight, or about 85% by weight, or about 86% by weight of the drug(s) is released; after about 18 hours from about 78% to about 94% by weight of the drug(s) is released, thus including all values, ranges and subranges therebetween, for example, from about 80% to about 94% by weight of the drug(s) is released, or from about 90% to about 94% by weight of the drug(s) is released, or about 91% by weight, about 92% by weight, about 93% by weight, or about 94% by weight of the drug is released, and after about 24 hours about 95% by weight of the drug(s) is released; or about 96% by weight, or about 97% by weight, or about 98% by weight, or about 99% by weight, or about 100% by weight of the drug(s) is released.

In at least one implementation of the first aspect, the composition provides an in vitro dissolution profile using a USP II paddle apparatus, water, and a rotation of 100 rpm, such that after about 2 hours, from about 0% to about 19% by weight of the drug(s) is released, including about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, thus including all values, ranges and subranges therebetween, for example, from about 5% to about 19% by weight of the drug(s) is released, or from about 10% to about 19% by weight of the drug(s) is released, or from about 15% to about 19% by weight of the drug(s) is released; after about 4 hours, from about 0% to about 69% by weight of the drug(s) is released, thus including all values, ranges, and subranges therebetween, for example, from about 5% to about 69% by weight of the drug(s) is released, or from about 10% to about 69% by weight of the drug(s) is released, or from about 15% to about 69% by weight of the drug(s) is released, of from about 20% to about 69% by weight of the drug(s) is released, or from about 25% to about 69% by weight of the drug(s) is released, or from about 30% to about 69% by weight of the drug(s) is released, or from about 35% to about 69% by weight of the drug(s) is released, or from about 40% to about 69% by weight of the drug(s) is released, or from about 45% to about 69% by weight of the drug(s) is released, or from about 50% to about 69% by weight of the drug(s) is released, or from about 55% to about 69% by weight of the drug(s) is released, or from about 60% to about 69% by weight of the drug(s) is released, or from about 65% to about 69% by weight of the drug(s) is released; after about 8 hours, about 95% by weight of the drug(s) is released, thus including all values, ranges and subranges therebetween, for example, from about 5% to about 95% by weight of the drug(s) is released, or from about 10% to about 95% by weight of the drug(s) is released, or from about 15% to about 95% by weight of the drug(s) is released, of from about 20% to about 95% by weight of the drug(s) is released, or from about 25% to about 95% by weight of the drug(s) is released, or from about 30% to about 95% by weight of the drug(s) is released, or from about 35% to about 95% by weight of the drug(s) is released, or from about 40% to about 95% by weight of the drug(s) is released, or from about 45% to about 95% by weight of the drug(s) is released, or from about 50% to about 95% by weight of the drug(s) is released, or from about 55% to about 95% by weight of the drug(s) is released, or from about 60% to about 95% by weight of the drug(s) is released, or from about 65% to about 95% by weight of the drug(s) is released, or from about 70% to about 95% by weight of the drug is released, or from about 75% to about 95% by weight of the drug(s) is released, or from about 80% to about 95% by weight of the drug(s) is released, or from about 85% to about 95% by weight of the drug(s) is released, or from about 90% to about 95% by weight of the drug(s) is released; and after about 12 hours, at least about 90%, or about 91%, or about 92%, or about 93%, or about 94%, or about 95%, or about 96%, or about 97%, or about 98%, or about 99%, or about 100% by weight of the drug(s) is released.

In at least one implementation of the first aspect, the drug(s) is selected from the group consisting of bupropion, carvedilol, citalopram, diltiazem, fluoxetine, metoprolol, pramipexole, quetiapine, ramipril, rivastigmine, rosiglitazone, sumatriptan, topiramate, tramadol, venlafaxine, zolpidem, memantine, dexmethylphenidate, and mixtures thereof.

In at least one implementation of the first aspect, the core is a LIQUIFLASH® microparticle.

In at least one implementation of the first aspect, the core further comprises at least one spheronising agent. In at least one embodiment, the spheronising agent is present in an amount of from about 5% to about 99.9% by weight of the core, thus including all values, ranges and subranges therebetween, thus including, for example, from about 10% to about 99.9% by weight of the core, from about 20% to about 99.9% by weight of the core, from about 30% to about 99.9% by weight of the core, from about 40% to about 99.9% by weight of the core, from about 50% to about 99.9% by weight of the core, from about 60% to about 99.9% by weight of the core, from about 70% to about 99.9% by weight of the core, from about 80% to about 99.9% by weight of the core, or from about 90% to about 99.9% by weight of the core; and the drug(s) is present in an amount of from about 0.1% to about 95% by weight of the core, including all ranges, values and subranges therebetween, thus including, for example, from about 1% to about 95% by weight of the core, or from about 5% to about 95% by weight of the core, or from about 10% to about 95% by weight of the core, or from about 20% to about 95% by weight of the core, or from about 30% to about 95% by weight of the core, or from about 40% to about 95% by weight of the core, or from about 50% to about 95% by weight of the core, or from about 60% to about 95% by weight of the core, or from about 70% to about 95% by weight of the core, or from about 80% to about 95% by weight of the core, or from about 90% to about 95% by weight of the core. In at least one embodiment, the spheronising agent is glyceryl monostearate.

In at least one implementation of the first aspect, the osmotic agent is soluble in aqueous and biological fluids. In at least one embodiment, the osmotic agent is sodium chloride.

In at least one implementation of the first aspect, the osmotic deposition vehicle is a low molecular weight hydroxypropylmethylcellulose polymer. In at least one embodiment, the osmotic deposition vehicle is PHARMACOAT® 606.

In at least one implementation of the first aspect, the osmotic deposition vehicle is present in an amount of from about 1% to about 99% by weight of the osmotic subcoat, including all values, ranges and subranges therebetween, thus including, for example, from about 5% to about 99% by weight of the osmotic subcoat, or from about 10% to about 99% by weight of the osmotic subcoat, or from about 20% to about 99% by weight of the osmotic subcoat, or from about 30% to about 99% by weight of the osmotic subcoat, or from about 40% to about 99% by weight of the osmotic subcoat, or from about 50% to about 99% by weight of the osmotic subcoat, or from about 60% to about 99% by weight of the osmotic subcoat, or from about 70% to about 99% by weight of the osmotic subcoat, or from about 80% to about 99% by weight of the osmotic subcoat, or from about 90% to about 99% by weight of the osmotic subcoat; and the osmotic agent is present in an amount of from about 1% to about 99% by weight of the osmotic subcoat, including all values, ranges and subranges therebetween, thus including, for example, from about 5% to about 99% by weight of the osmotic subcoat, or from about 10% to about 99% by weight of the osmotic subcoat, or from about 20% to about 99% by weight of the osmotic subcoat, or from about 30% to about 99% by weight of the osmotic subcoat, or from about 40% to about 99% by weight of the osmotic subcoat, or from about 50% to about 99% by weight of the osmotic subcoat, or from about 60% to about 99% by weight of the osmotic subcoat, or from about 70% to about 99% by weight of the osmotic subcoat, or from about 80% to about 99% by weight of the osmotic subcoat, or from about 90% to about 99% by weight of the osmotic subcoat.

In at least one implementation of the first aspect, the drug release is substantially unaffected by any presence of the osmotic deposition vehicle.

In at least one implementation of the first aspect, the drug release is substantially unaffected by compression of the coated cores into a tablet.

In at least one implementation of the first aspect, the modified release coating excludes a pore former.

In at least one implementation of the first aspect, the composition excludes a sealcoat.

In at least one implementation of the first aspect, the modified release coating includes at least one polymer. In at least one embodiment, the polymer is an acrylate dispersion. In at least one embodiment, the polymer in the modified release coating is EUDRAGIT® NE30D.

In at least one implementation of the first aspect, the core is coated with at least one taste-masking coating. In at least one embodiment, the taste-masking coating contains at least one cellulosic polymer.

According to another aspect of the present invention, there is provided a modified release pharmaceutical composition for oral administration suitable for once daily dosing comprising (i) a core including at least one drug; (ii) an osmotic subcoat including at least one osmotic agent and at least one osmotic deposition vehicle, wherein the osmotic subcoat at least partially or fully surrounds the core; (iii) a modified release coating that at least partially or fully surrounds the osmotic subcoat; and (iv) an additional overcoat that includes at least one drug, wherein the additional overcoat at least partially or fully surrounds the modified release coating. Thus, the modified release coating could surround from about 1% to about 100%, including all values, ranges and subranges therebetween, thus including, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, or from about 95% to about 100% of the osmotic subcoat. Similarly, the osmotic subcoat could surround from about 1% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, or from about 95% to about 100% of the core Finally, the additional overcoat could surround from about 1% to about 100%, from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, or from about 95% to about 100% of the modified release coating.

In certain embodiments the modified release dosage form can include an osmotic dosage form, a swellable dosage form, a swellable and erodable dosage form, an erodable dosage form, an insoluble dosage form, a hydrophobic matrix dosage form, a hydrophilic dosage form, a modified release coat, an insoluble coat, a swellable coat, an erodable coat, a swellable and erodable coat, an extended release dosage form, a delayed release dosage form, a sustained release dosage form, a prolonged-release dosage form, a bi-phasic release dosage form, an immediate release matrix core coated with at least one modified release coat, an immediate release matrix core coated with at least one aqueous controlled-release coat, an immediate release matrix core coated with at least one aqueous swellable coat, an immediate release matrix core coated with at least one swellable and erodable coat, an immediate release matrix core coated with at least one erodable coat, or any combination thereof.

In at least one embodiment the modified release dosage form comprises at least one delayed release coat.

In at least one embodiment the modified release dosage form comprises at least one modified release coat, which comprises a material that is soluble or slowly dissolving in intestinal juices, substantially pH neutral or basic fluids or fluids having a pH higher than gastric fluid, but for the most part insoluble in gastric juices or acidic fluids.

In at least one embodiment the modified release dosage form comprises at least one delayed release coat, which comprises at least one water-insoluble water-permeable film-forming polymer and at least one water-soluble polymer.

In at least one embodiment the modified release dosage form comprises at least one modified release coat, which comprises at least one water-insoluble water-permeable film-forming polymer, at least one water-soluble polymer, and at least one plasticizer.

In at least one embodiment the modified release dosage form comprises at least one modified release coat, which coat comprises at least one enteric polymer.

In at least one embodiment the modified release coat provides extended release of the drug(s).

In at least one embodiment the modified release coat provides sustained release of the drug(s).

In at least one embodiment the modified release coat provides delayed release of the drug(s).

In at least one embodiment the modified release dosage form comprises at least one modified release coat, which comprises at least one aqueous dispersion of a neutral ester copolymer without any functional groups, a poly glycol having a melting point greater than about 55° C., and one or more pharmaceutically acceptable excipients and is cured at a temperature at least equal to or greater than the melting point of the poly glycol.

In at least one embodiment the modified release dosage form comprises at least one delayed release coat, which coat comprises at least one pH dependent polymer.

In at least one embodiment the modified release dosage form comprises two or more coats, wherein one coat comprises a modified release coat.

In at least one embodiment the modified release dosage form comprises two or more coats, wherein one coat comprises a delayed-release coat.

In at least one embodiment the modified release dosage form comprises at least one non-functional soluble coat.

In at least one embodiment the modified release dosage form comprises at least one insoluble coat.

In at least one embodiment the modified release dosage form comprises at least one swellable coat.

In at least one embodiment the modified release dosage form comprises at least one erodable coat.

In at least one embodiment the modified release dosage form comprises at least one swellable and erodable coat.

In at least one embodiment the modified release dosage form comprises at least one means for the exit of drug from the modified release dosage form.

In at least one embodiment the modified release dosage form comprises at least one means for increasing the hydrostatic pressure of the modified release dosage form.

In at least one embodiment the modified release dosage form comprises at least one means for forcibly dispensing the drug from the modified release dosage form.

In at least one embodiment the modified release dosage form is in the form of a tablet.

In at least one embodiment the modified release dosage form is in the form of a capsule.

In at least one embodiment the modified release dosage form is in the form of a microparticle.

In at least one embodiment the modified release dosage form is in the form of a plurality of microparticles, wherein each microparticle comprises an osmotic subcoat.

In at least one embodiment the modified release dosage form is in the form of a plurality of microparticles, wherein each microparticle comprises an osmotic subcoat and a modified release overcoat.

In at least one embodiment the modified release dosage form is in the form of a plurality of microparticles, wherein each microparticle comprises an insoluble polymer.

In at least one embodiment the modified release dosage form comprises at least one modified release matrix core.

In at least one embodiment the modified release dosage form comprises at least one swellable matrix core.

In at least one embodiment the modified release dosage form comprises at least one erodable matrix core.

In at least one embodiment the modified release dosage form comprises at least one swellable and erodable matrix core.

In at least one embodiment the modified release dosage form comprises at least one insoluble matrix core.

In at least one embodiment the modified release dosage form comprises at least one insoluble polymer matrix core.

In at least one embodiment the modified release dosage form comprises at least one hydrophobic matrix core.

In at least one embodiment the modified release dosage form comprises at least one hydrophilic matrix core.

In at least one embodiment the modified release dosage form comprises at least one combination of a hydrophobic and hydrophilic matrix core.

In at least one embodiment the modified release dosage form comprises at least one immediate release matrix core coated with at least one modified release coat.

In at least one embodiment the modified release dosage form comprises at least one immediate release matrix core coated with at least one aqueous controlled-release coat.

In at least one embodiment the modified release dosage form comprises at least one lipid or wax dosage form.

In at least one embodiment the modified release dosage form comprises at least one unitary core.

In at least one embodiment the modified release dosage form comprises at least one delayed release dosage form.

In at least one embodiment the modified release dosage form comprises at least one extended release dosage form.

The present invention will be further understood from the following detailed description with reference to the following figures in which:

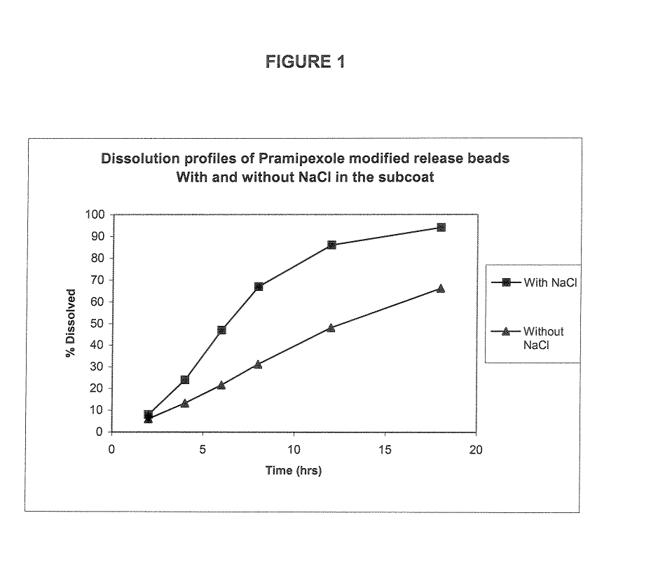

FIG. 1 illustrates the effect of the osmotic subcoat on rate and extent of drug release from Pramipexole modified release microparticles.

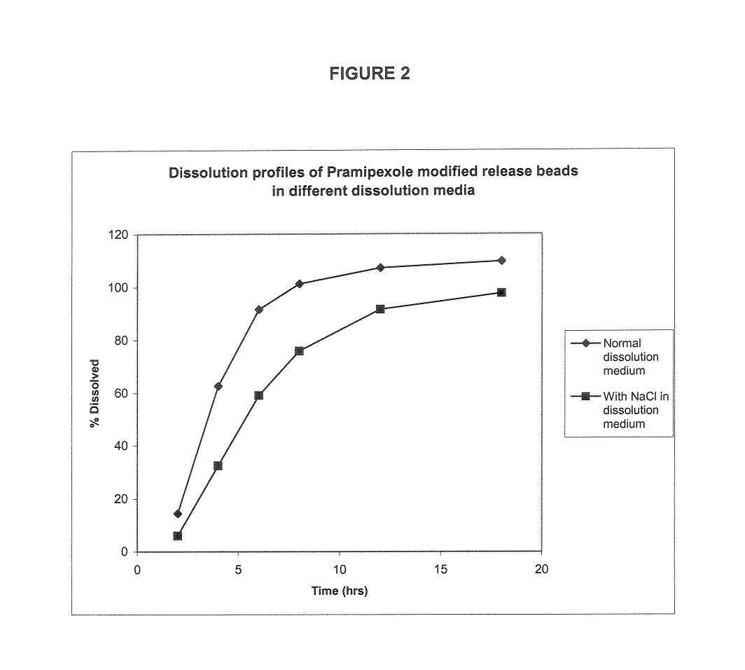

FIG. 2 compares release profiles of Pramipexole modified release microparticles in different dissolution media.

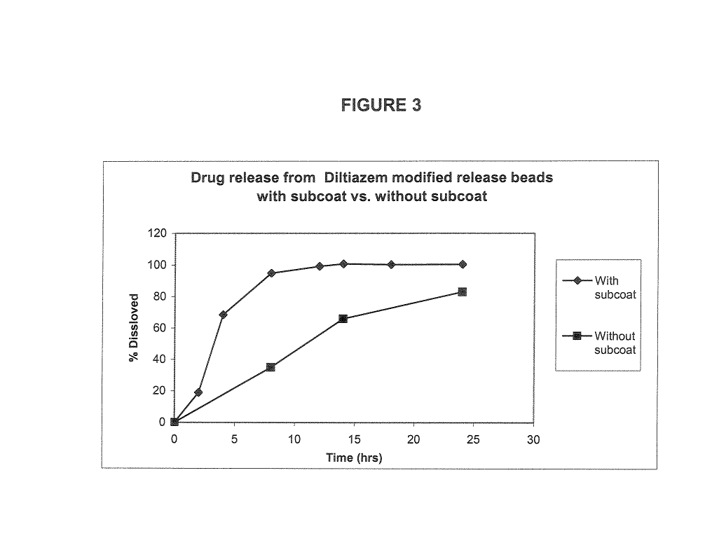

FIG. 3 illustrates the effect of the osmotic subcoat on rate and extent of drug release from Diltiazem modified release microparticles.

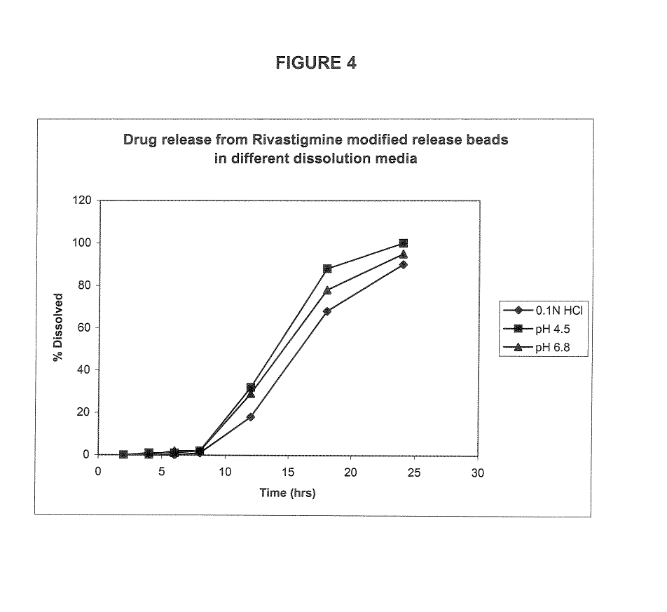

FIG. 4 illustrates lagtimes achieved from Rivastigmine delayed release microparticles in different dissolution media.

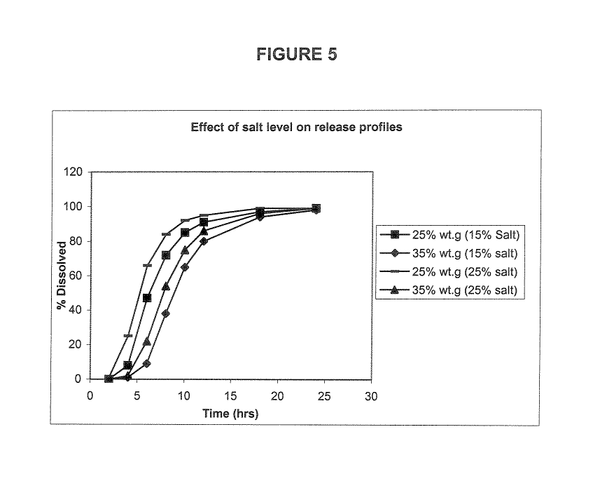

FIG. 5 illustrates the effect of the level of osmotic agent on release profiles of Rivastigmine modified release microparticles.

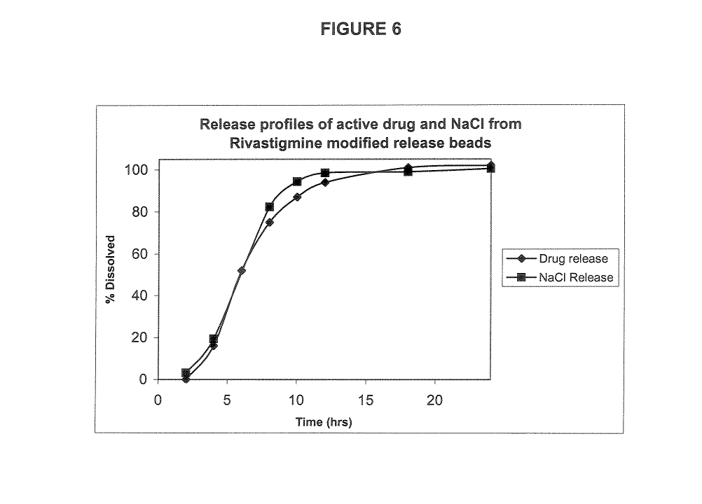

FIG. 6 compares release profiles of osmotic agent and Rivastigmine from Rivastigmine delayed release microparticles.

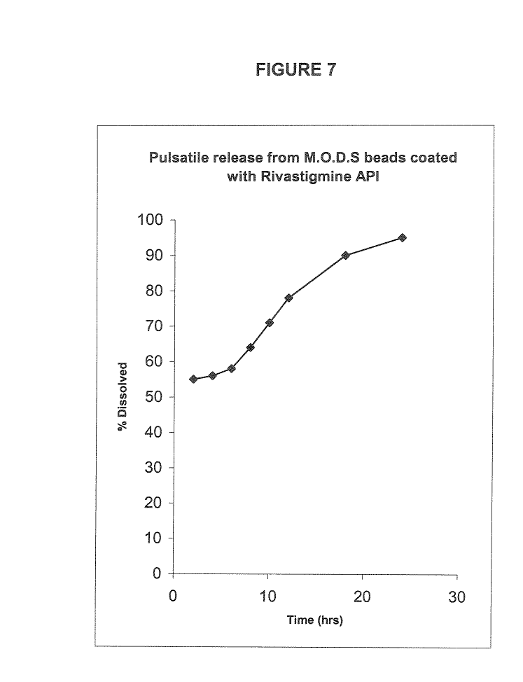

FIG. 7 illustrates pulsatile drug release from Rivastigmine delayed release microparticles coated with an additional overcoat containing Rivastigmine.

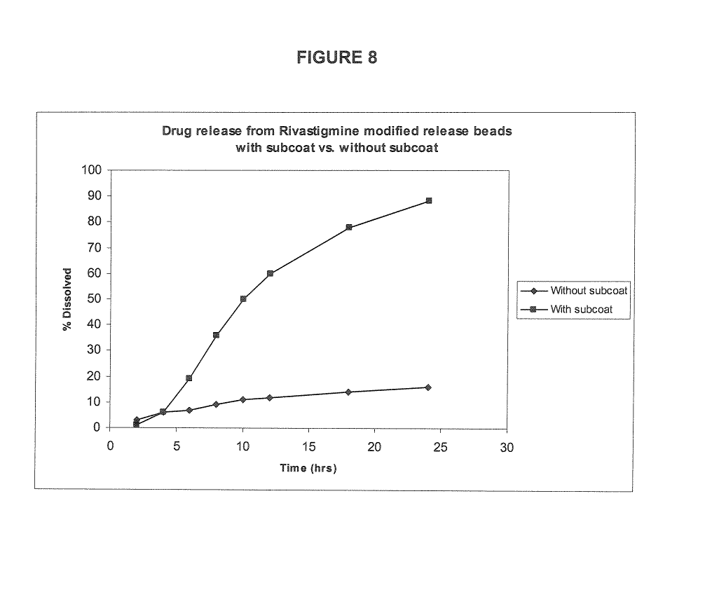

FIG. 8 illustrates the effect of the osmotic subcoat on rate and extent of drug release from Rivastigmine modified release microparticles (The modified release overcoat was obtained by applying SURELEASE® dispersion onto Rivastigmine microparticles)

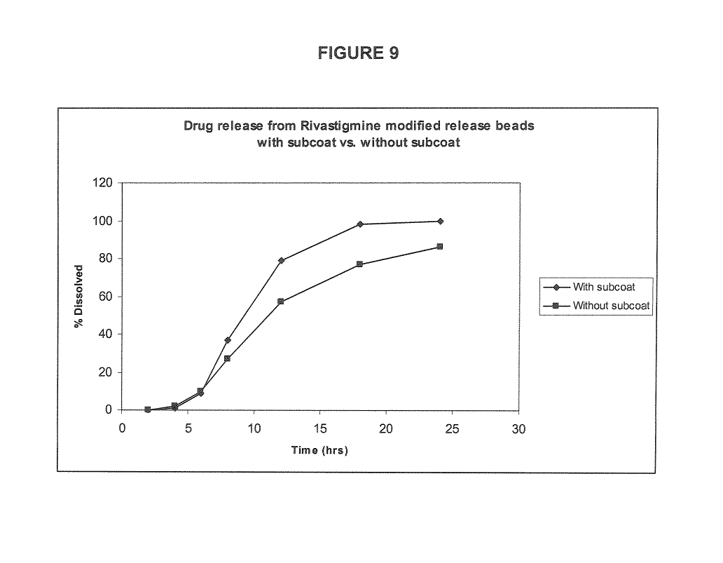

FIG. 9 illustrates the effect of the osmotic subcoat on rate and extent of drug release from Rivastigmine delayed release microparticles.

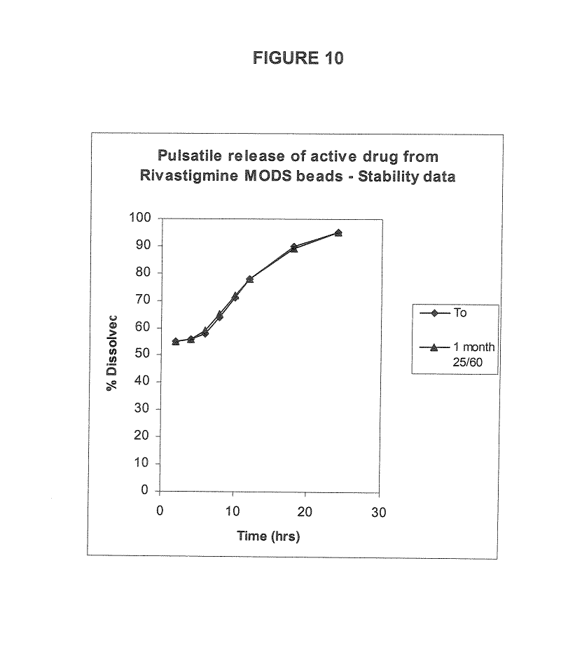

FIG. 10 illustrates pulsatile drug release from Rivastigmine delayed release microparticles having an additional overcoat containing Rivastigmine, and the stability of the composition after storage at 1 month at 25° C. and 60% RH.

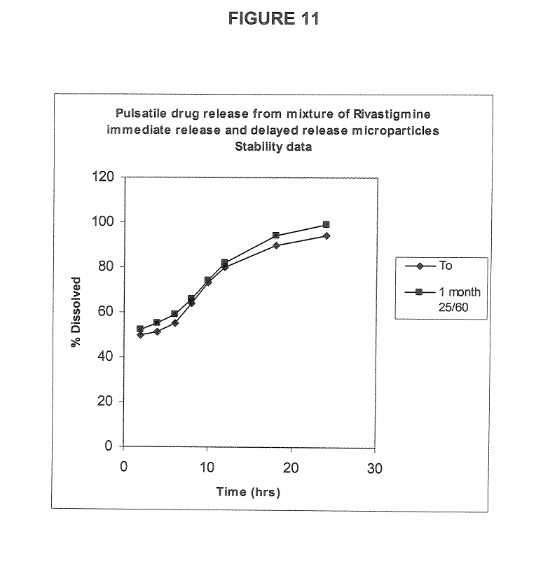

FIG. 11 illustrates pulsatile drug release from a dosage form containing a mixture of Rivastigmine immediate release microparticles and Rivastigmine delayed release microparticles, and the stability of the composition after storage at 1 month at 25° C. and 60% RH.

The following definitions are provided in order to more specifically describe the invention. Otherwise all terms are to be accorded their ordinary meaning as they would be construed by one of ordinary skill in the art, i.e. pharmaceutical drug formulations.

The terms “drug”, “active drug”, “active”, “active agent” or “active pharmaceutical agent” as used herein are used interchangeably in this application, and are defined to mean a molecule or ion, including those appended portions of the molecule or ion that cause the molecule or ion to be an ester, and/or a salt (including a salt with hydrogen or coordination bonds); and further encompasses any active pharmaceutical ingredient (“API”), including its pharmaceutically acceptable organic and/or inorganic salts (non limiting examples of which include the hydrochloride salts, the hydrobromide salts, the hydroiodide salts, and the saccharinate salts), as well as the anhydrous, hydrated, solvated forms and/or isoforms of the API, prodrugs of the active pharmaceutical ingredient, and individually optically active enantiomers of the API, as well as mixtures of these enantiomers, including racemic mixtures, and where appropriate, diastereomers and/or mixtures of diastereomers. A prodrug is a molecule or ion whose biological activity is altered and/or increased after being introduced into the body, for example, by alteration of the molecule or ion, for example by oxidation and/or amination and/or deamination and/or reduction and/or conjugation and/or acylation and/or alkylation and/or hydrolysis and/or dealkylation, and/or rearrangement of the molecule or ion, and/or or removal of some part of the molecule or ion.

The term “high-solubility drug” as used herein refers to drugs wherein about ten parts of water or less can be used to dissolve one part of drug.

The term “low-solubility drug,” as used herein refers to drugs wherein about thirty parts of water or more is used to dissolve one part of drug.

The term “normal-solubility drug” as used herein refers to drugs wherein from more than about ten parts of water to less than about thirty parts of water can be used to dissolve one part of drug.

The term “high-dose drug” as used herein refers to drugs that are dosed at about 200 mg or more per dosage form including about 200 mg, about 300 mg, about 400 mg, about 500 mg, about 600 mg, about 700 mg, about 800 mg, about 900 mg, about 1000 mg, and even higher doses, including all values, ranges and subranges between about 200 mg and about 1000 mg, and including all values, ranges and subranges between about 200 mg and about 10,000 mg.

The term “low-dose drug” as used herein refers to drugs that are dosed at about 20 mg or less per dosage form including about 20 mg, about 19 mg, about 18 mg, about 17 mg, about 16 mg, about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, about 11 mg, about 0.5 mg, about 0.1 mg, or even a lower dose, including all values, ranges and subranges therebetween.

The term “normal-dose drug” as used herein refers to drugs that are dosed at from more than about 20 mg to less than about 200 mg per dosage form, including more than about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, and about 200 mg, including all ranges, subranges and values therebetween, thus including, for example, from about 30 mg to less than about 200 mg, or from about 40 mg to less than about 200 mg, or from about 50 mg to less than about 200 mg, or from about 60 mg to less than about 200 mg, or from about 70 mg to less than about 200 mg, or from about 80 mg to less than about 200 mg, or from about 90 mg to less than about 200 mg, or from about 100 mg to less than about 200 mg, or from about 110 mg to less than about 200 mg, or from about 120 mg to less than about 200 mg, or from about 130 mg to less than about 200 mg, or from about 140 mg to less than about 200 mg, or from about 150 mg to less than about 200 mg, or from about 160 mg to less than about 200 mg, or from about 170 mg to less than about 200 mg, or from about 180 mg to less than about 200 mg, or from about 190 mg to less than about 200 mg.

The term “formulation” or “composition” as used herein refers to the drug in combination with at least one pharmaceutically acceptable carrier and/or at least one additional inert ingredient.

The term “microparticle”, as used herein is defined to mean a drug formulation in discrete particulate form, and is interchangeable with the terms “microspheres”, “spherical particles”, “microcapsules”, “particles”, “multiparticulates”, “granules”, “spheroids”, beads”, spherules and “pellets”.

The term “tablet” as used herein refers to a single dosage form, e.g. the single entity containing at least one drug that is administered to a subject, or a subject in need thereof. Preferably, the subject or subject in need thereof is a warm blooded animal. More preferably, the subject or subject in need thereof is a human. The term “tablet” also includes a tablet that may be the combination of one or more “microparticles”.

The term “dosage form” as used herein is defined to mean a pharmaceutical preparation or system in which dose(s) of drug(s) are included. A dosage form can comprise, for example, at least one modified release dosage form, at least one osmotic dosage form, at least one erosion modified release dosage form, at least one dissolution modified release dosage form, at least one diffusion modified release dosage form, at least one modified release matrix core, at least one modified release matrix core coated with at least one modified release coat, at least one enteric coated dosage form, at least one dosage form surrounded by at least one osmotic subcoat, capsules, minitablets, caplets, uncoated microparticles, microparticles coated with at least one modified release coat, or any combination thereof.

“Modified release dosage forms” as used herein is defined (e.g. as by the USP) as those whose drug release characteristics of time course and/or location are chosen to accomplish therapeutic or convenience objectives not offered by conventional, immediate release dosage forms. The rate of release of the drug from a modified release dosage form is controlled by features of the dosage form and/or in combination with physiologic or environmental conditions rather than by physiologic or environmental conditions alone. The modified release dosage forms of certain embodiments of the invention can be contrasted to conventional immediate release dosage forms which typically produce large maximum/minimum plasma drug concentrations (Cₘₐₓ/Cₘᵢₙ) due to rapid absorption of the drug into the body (i.e., in-vivo, relative to the drug's therapeutic index; i.e., the ratio of the maximum drug concentration needed to produce and maintain a desirable pharmacological response). In conventional, immediate release dosage forms, the drug content is released into the gastrointestinal tract within a short period of time, and plasma drug levels peak shortly after dosing. The design of conventional immediate release dosage forms is generally based on getting the fastest possible rate of drug release, and therefore absorbed, often at the risk of creating undesirable dose related side effects. The modified release dosage forms of certain embodiments of the invention, on the other hand, improve the therapeutic value of the drug by reducing the ratio of the maximum/minimum plasma drug concentration (Cₘₐₓ/Cₘᵢₙ) while maintaining drug plasma levels within the therapeutic window. The modified release dosage forms of certain embodiments of the invention attempt to deliver therapeutically effective amounts of the drug as a dose administered at least once-daily so that the ratio Cₘₐₓ/Cₘᵢₙ in the plasma at steady state is less than the therapeutic index, and to maintain drug levels at constant effective levels to provide therapeutic benefit over a period of time (e.g. 24-hour period). The modified release dosage forms of certain embodiments of the invention, therefore, avoid large peak-to-trough fluctuations normally seen with conventional or immediate release dosage forms and can provide a substantially flat serum concentration curve throughout the therapeutic period. Modified-release dosage forms of certain embodiments can be designed to provide a quick increase in the plasma concentration of the drug which remains substantially constant within the therapeutic range of the drug for a period of time (e.g. at least a 24-hour period). Alternatively, modified-release dosage forms of certain embodiments can be designed to provide a quick increase in the plasma concentration of the drug, which although may not remain constant, declines at rate such that the plasma concentration remains within the therapeutic range for a period of time (e.g. at least a 24-hour period). The modified release dosage forms of certain embodiments of the invention can be constructed in many forms. The USP considers that the terms controlled release, prolonged release and sustained release are interchangeable. Accordingly, the terms “modified-release”, controlled-release”, “control-releasing”, “rate-controlled release”, “prolonged-release”, and “sustained-release” are used interchangeably herein. For the discussion herein, the definition of the term “modified-release” encompasses the scope of the definitions for the terms “extended release”, “enhanced-absorption”, “controlled release”, “sustained release”, and “delayed release”.

“Controlled release dosage forms”, “control-releasing dosage forms”, “rate-controlled release dosage forms”, or dosage forms which exhibit a “controlled release” of the drug as used herein are used interchangeably in this application and are defined to mean a dosage form, which releases the drug gradually or in a controlled manner per unit time in-vivo. The rate of release of the drug from a controlled release dosage form is controlled by features of the dosage form and/or in combination with physiologic or environmental conditions rather than by physiologic or environmental conditions alone. The controlled release dosage forms of certain embodiments of the invention can be contrasted to immediate release dosage forms which typically produce large maximum/minimum plasma drug concentrations (Cₘₐₓ/Cₘᵢₙ) due to rapid absorption of the drug into the body i.e., in-vivo, relative to the drug's therapeutic index i.e., the ratio of the maximum drug concentration needed to produce and maintain a desirable pharmacological response. In immediate release dosage forms, the drug content is released into the gastrointestinal tract within a short period of time, and plasma drug levels peak shortly after dosing. The design of immediate release dosage forms is generally based on getting the fastest possible rate of drug release, and therefore absorbed, often at the risk of creating undesirable dose related side effects. The controlled release dosage forms of certain embodiments of the invention, on the other hand, improve the therapeutic value of the drug by reducing the ratio of the maximum/minimum plasma drug concentration (Cₘₐₓ/Cₘᵢₙ) while maintaining drug plasma levels within the therapeutic window. The controlled release dosage forms of certain embodiments of the invention attempt to deliver therapeutically effective amounts of the drug, racemic mixtures thereof, enantiomers thereof, pharmaceutically acceptable salts thereof, and combinations thereof as a dose administered at least once-daily so that the ratio Cₘₐₓ/Cₘᵢₙ in the plasma at steady state is less than the therapeutic index, and to maintain drug levels at constant effective levels to provide therapeutic benefit over a period of time (e.g. a 24-hour period). The controlled release dosage forms of certain embodiments of the invention, therefore, avoid large peak-to-trough fluctuations normally seen with immediate release dosage forms and provide a substantially flat serum concentration curve throughout the therapeutic period. The controlled release dosage forms of certain embodiments of the invention can be constructed in many forms known to one of ordinary skill in the drug delivery arts and described in the prior art such as for example, osmotic dosage forms, multiparticulate dosage forms, and gastric retention dosage forms.

“Sustained-release dosage forms” or dosage forms which exhibit a “sustained-release” of the drug as used herein is defined to mean dosage forms administered at least once-daily that provide a release of the drug sufficient to provide a therapeutic dose soon after administration, and then a gradual release over an extended period of time such that the sustained-release dosage form provides therapeutic benefit over a period of time (e.g. a 24-hour period, or over a period of time ranging from about 1 to about 24 hours).

“Extended-release dosage forms” or dosage forms which exhibit an “extended release” of the drug as used herein is defined to mean dosage forms administered at least once-daily that release the drug slowly, so that plasma concentrations of the drug are maintained at a therapeutic level for an extended period of time such that the sustained-release dosage form provides therapeutic benefit over a period of time (e.g. a 24-hour period, or over a period or time ranging from about 1 to about 24 hours).

“Delayed-release dosage forms” or dosage forms which exhibit a “delayed release” of the drug as used herein is defined to mean dosage forms administered at least once-daily that do not effectively release drug immediately following administration but at a later time. Delayed-release dosage forms provide a time delay prior to the commencement of drug-absorption. This time delay can be referred to as “lag time” and should not be confused with “onset time” which represents latency, that is, the time required for the drug to reach minimum effective concentration.

The terms “osmotic dosage form”, “osmotic delivery device”, “modified release osmotic dosage form” or “controlled release osmotic dosage form” as used herein are used interchangeably in this application, and are defined to mean dosage forms which dispense the drug(s) all or in part as a result of the presence of an osmotic agent in the osmotic subcoat driving solvent (e.g. water, dissolution media, gastric fluid, intestinal fluid, or mixtures thereof) into the core, which subsequently facilitates the release of drug from the core. Osmotic dosage forms encompass dosage forms that include an osmotic subcoat, and dosage forms that can be coated with a modified release overcoat.

The term “osmosis” as used herein refers to the flow of a solvent through a selectively-permeable membrane (e.g. modified release overcoat) from a region of high solvent potential to a region of low solvent potential. The selectively-permeable membrane is permeable to the solvent, but not to the solute, resulting in a pressure gradient across the membrane. Non-limiting examples of selectively-permeable membranes include semipermeable membranes, and microporous, asymmetric membranes (which can be permeable, semipermeable, perforated, or unperforated) and can deliver the drug(s) by osmotic pumping, diffusion or the combined mechanisms of diffusion and osmotic pumping. Thus, in principle, osmosis controlled release of the drug(s) involves osmotic transport of an aqueous media into the osmotic dosage form followed by dissolution of the drug(s) and the subsequent transport of the saturated solution of the drug by osmotic pumping of the solution through at least one passageway in the selectively-permeable membrane and/or by diffusion through the selectively-permeable membrane.

“Osmotic pressure gradient” as used herein is defined to mean the difference in hydrostatic pressure produced by a solution in a space divided by a selectively-permeable membrane due to a differential in the concentrations of solute.

The terms “osmotic agent”, “osmagent”, “osmotically effective solute”, “osmotic enhancer” “osmotically effective compounds”, “osmotic solutes”, “osmotically active agent”, “osmopolymer” and “osmotic fluid imbibing agents” as used herein are used interchangeably, and define any material that is soluble (i.e. can be partially or totally solubilized) or swellable in a solvent (e.g. water) that enters the composition, and which exhibits an osmotic pressure gradient across the selectively-permeable membrane (e.g. modified release overcoat), thus increasing the hydrostatic pressure inside the osmotic dosage form.

The term “osmotic deposition vehicle” as used herein is defined to mean a carrier for the osmotic agent in the osmotic subcoat. The osmotic deposition vehicle can be any type of hydrophilic polymer.

The term “osmotic subcoat” as used herein is defined to mean a functional coat that comprises at least one osmotic agent and at least one osmotic deposition vehicle in amounts sufficient to achieve an osmotic pressure gradient across one or more outer coats (e.g. modified release overcoat and/or additional overcoat) for the transport of solvent or aqueous fluid (e.g. water, dissolution media, gastric, intestinal fluid, or mixtures thereof) from the external environment of use into the core, and the transport of drug solution from the core out into the external environment of use. The osmotic subcoat can modify the rate and/or extent of release of drug from the core. For example, the osmotic subcoat can provide increased release and/or substantially full release of at least one drug from the core. The osmotic subcoat at least partially surrounds the core, and is in turn at least partially surrounded by at least one outer coat (e.g. modified release overcoat and/or additional overcoat). The osmotic subcoat can optionally comprise additional materials that can alter the functionality of the osmotic subcoat.

The terms “modified release overcoat”, “controlled release coat”, “control releasing coat”, and “rate-controlling coat” as used herein are used interchangeably in this application, and are defined to mean a functional coat which comprises at least one modified release polymer. Non-limiting examples of modified release polymers include pH independent polymers, pH dependent polymers (such as for example enteric or reverse enteric types), soluble polymers, insoluble polymers, lipids, lipidic materials, and mixtures thereof. When applied onto a dosage form, the modified release overcoat can modify (e.g. slow) the rate of release of the drug. For example, the modified release overcoat can be designed such that when the modified release overcoat is applied onto a dosage form, the dosage form in conjunction with the modified release overcoat, exhibits a “modified-release”, “controlled-release”, “sustained-release”, “extended-release” and/or “delayed-release” profile. Combinations thereof are permissible. The “modified release overcoat” can optionally comprise additional materials that can alter the functionality of the modified release overcoat. The term “modified release” is interchangeable with the terms “controlled release”, “control releasing” and “rate controlling”. The term “overcoat” is interchangeable with the terms “coat” and “coating”.

The terms “moisture barrier” and “moisture barrier coat” as used herein are used interchangeably and are defined to mean a coating which impedes or retards the absorption of moisture. Some drugs are susceptible to decomposition over time under high humidity conditions. The proportion of the components of the moisture barrier and the amount of the moisture barrier applied onto the modified release overcoat is such that the moisture barrier does not fall within the USP definition and requirement for an enteric coat. Suitably, the moisture barrier is comprised of an enteric and/or acrylic polymer, suitably an acrylic polymer, optionally a plasticizer, and a permeation enhancer. The permeation enhancer is a hydrophilic substance, which allows water to enter without physical disruption of the coating. The moisture barrier can additionally contain other conventional inert excipients, which may improve processing of the extended-release formulation described herein.

The term “enteric coat” as used herein is defined to mean a coating or barrier applied to a dosage form that can control the location in the digestive system where the drug is absorbed. For example, an enteric coating can be used to: (i) protect the drug from the destructive action of the enzymes or low pH environment of the stomach; (ii) prevent nausea or bleeding associated with the irritation of the gastric mucosa by the drug; and/or (iii) deliver the drug in an undiluted form in the intestine. Based on these criteria, in certain embodiments, the enteric coated dosage form can be regarded as a type of delayed release dosage form. They differ from sustained release dosage forms in that with sustained release dosage forms, the drug release is extended over a period of time to maintain therapeutic blood levels and to decrease the incidence of side effects caused by a rapid release; whereas, with enteric coatings, the primary objective is to confine the release of the drug to a predetermined region of the gastrointestinal tract. Enteric coatings work by presenting a surface that is substantially stable at acidic pH, but breaks down at higher pH to allow release of the drug in the intestine.

The term “enteric polymer” as used herein is defined to mean a polymeric substance that when used in an enteric coat formulation, is substantially insoluble and/or substantially stable under acidic conditions exhibiting a pH of less than about 5 to about 1, including a pH of about 4, or a pH of about 3, or a pH of about 2, or a pH of about 1, including all values, ranges and subranges therebetween, and which are substantially soluble or can decompose under conditions exhibiting a pH of about 5 or more, or about 5 to about 14, including a pH of about 6, or a pH of about 7, or a pH of about 8, or a pH of about 9, or a pH of about 10, or a pH of about 11, or a pH of about 12, or a pH of about 13, or a pH of about 14, including all values, ranges and subranges therebetween. Non-limiting examples of such enteric polymers include carboxymethylethylcellulose, cellulose acetate phthalate, cellulose acetate succinate, methylcellulose phthalate, hydroxymethylethylcellulose phthalate, hydroxypropylmethylcellulose phthalate, hydroxypropylmethylcellulose acetate succinate, polyvinyl alcohol phthalate, polyvinyl butyrate phthalate, polyvinyl acetal phthalate, a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate, a copolymer of methacrylic acid/methyl methacrylate and mixtures thereof. Enteric polymers can be used individually or in combination with other hydrophobic or hydrophilic polymers in an enteric coat, a normal release matrix core, a controlled release matrix core, and/or in a modified release coat. Enteric polymers can be combined with other pharmaceutically acceptable excipients to either facilitate processing of a coat comprising the enteric polymer or to alter the functionality of the coat.

The term “functional coat” as used herein is defined to mean a coating that affects the rate of release in vitro or in vivo of the drug.

The term “non-functional coat” as used herein is defined to mean a coating that does not substantially affect the rate of release in-vitro or in-vivo of the drug, but can enhance the chemical, biological, physical stability characteristics, or the physical appearance of the modified release dosage form.

The term “outer coat” as used herein in this application is defined to mean a functional coat that at least partially surrounds (i.e. is located outside of) the osmotic subcoat. Non-limiting examples include a modified release overcoat, and an additional overcoat.

The terms “coat”, “coating” and “membrane” as used herein are used interchangeably and are defined to mean a functional coat or a non-functional coat.

“Sealcoat” as used herein refers to a coat that separates at least two different compartments (e.g. separates a drug-containing core and a functional coat) in a drug delivery system, and controls, reduces, minimizes and/or eliminates unfavourable interactions between ingredients of the at least two different compartments, and/or controls, reduces, minimizes and/or eliminates unfavourable interactions between the ingredients of the core and the dissolution medium which may have an undesirable effect upon the rate and extent of drug release from the dosage form. A non-limiting example of a sealcoat includes a polymer coat that separates a hygroscopic core and a drug-containing coat, wherein the sealcoat reduces any hygroscopic effect of the hygroscopic core on the stability of drug in the drug-containing coat. A further non-limiting example of a sealcoat includes a polymer coat that separates a microparticle core and a drug-containing coat, wherein the sealcoat reduces undesirable effects that the ingredients of the microparticle core can have on the rate and extent of drug release upon contact with the dissolution medium.

The term “core” as used herein is defined to mean a solid vehicle in which at least one drug is uniformly or non-uniformly dispersed. The core can be formed by methods and materials well known in the art, such as for example by compressing, fusing, or extruding the drug together with at least one pharmaceutically acceptable excipient. The core can be manufactured into, for example, a homogenous or non-homogenous unitary core, a multiparticle, or a plurality of microparticles compressed into a unitary core. Non-limiting examples of cores include microparticle cores, matrix cores, and osmotic cores. The core(s) can be coated with at least one osmotic subcoat, modified release overcoat, additional overcoat, semi-permeable coat or membrane, non-functional coat, or any combination thereof.

The terms “modified release matrix core” and “controlled release matrix core” as used herein are used interchangeably, and are defined to mean a core in which at least one drug is dispersed within a matrix which controls or delays the release of the drug over a 24-hour period so as to allow a composition comprising the modified release matrix core to be administered as a once-a-day composition. The release rate of the drug from the modified release matrix core can be modified by the porosity and tortuosity of the matrix, (i.e. its pore structure). The addition of pore-forming hydrophilic salts, solutes, or wicking agents can influence the release rate, as can the manipulation of processing parameters. For example, the compression force used in the manufacture of the modified release matrix core can alter the porosity of the matrix core and hence the rate of release of the drug. It will be understood by one of ordinary skill in the art of drug delivery that a more rigid matrix will be less porous and hence release the drug more slowly compared to a less rigid modified release matrix core. The modified release matrix core can comprise insoluble or inert matrix dosage forms, swellable matrix dosage forms, swellable and erodable matrix dosage form, hydrophobic matrix dosage forms, hydrophilic matrix dosage forms, erodable matrix dosage forms, reservoir dosage forms, or any combination thereof. The modified release matrix core can comprise at least one insoluble matrix, at least one swellable matrix, at least one swellable and erodable matrix, at least one hydrophobic matrix, at least one hydrophilic matrix, at least one erodable matrix, or a combination thereof in which the rate of release is slower than that of uncoated immediate-release dosage forms. Modified release matrix cores can be coated with at least one modified release coat to further slow the release of the drug from the modified release matrix core. Such coated modified release matrix cores can exhibit modified-release, controlled-release, sustained-release, extended-release, prolonged-release, bi-phasic release, delayed-release or combinations thereof of the drug. Modified release matrix cores can also be coated with a non-functional soluble coat.

The term “immediate-release matrix core” or “normal release matrix core”, as used herein are used interchangeably, and are defined to mean a core in which at least one drug is dispersed within a matrix, which matrix can be insoluble, soluble, swellable, erodable, or combinations thereof. The immediate-release matrix cores of certain embodiments do not comprise starch derivatives and water-soluble materials such as, for example, gelatin, polyvinylpyrrolidone, polyvinyl alcohol, hydroxypropylmethylcellulose, hydroxypropylcellulose, xanthan gum, carbomers, and caragheen. Immediate release matrix cores can be manufactured such that the release of the drug mimics the release rate of an uncoated non-matrix or immediate-release dosage form comprising the drug. The release rate from immediate release matrix cores can be slowed down, controlled, delayed or modified in conjunction with a modified release coat. In the absence of the modified release coat, the release of drug from a normal release matrix core is substantially immediate.

The term “plasticizer” as used herein includes any compounds capable of plasticizing or softening a polymer or a binder used in the present invention. The use of plasticizers is optional, and can be included in the dosage form to modify the properties and characteristics of the polymers used in the coat(s) or core of the dosage form for convenient processing during manufacture of the coat(s) and/or the core of the dosage form. Once the coat(s) and/or core have been manufactured, certain plasticizers can function to increase the hydrophilicity of the coat(s) and/or the core of the dosage form in the environment of use. During manufacture of the coat(s) and/or core, the plasticizer can lower the melting temperature or glass transition temperature (softening point temperature) of the polymer or binder. Plasticizers can broaden the average molecular weight of a polymer in which they are included thereby lowering its glass transition temperature or softening point. Plasticizers also can reduce the viscosity of a polymer. Plasticizers can impart some particularly advantageous physical properties to the dosage forms of the invention.

The terms “pore former”, “pore forming agent”, and “pore forming additive” as used herein are used interchangeably in this application, and are defined to mean an excipient that can be added to a coating (e.g. the modified release overcoat), wherein upon exposure to fluids in the environment of use, the pore former dissolves or leaches from the coating to form pores, channels or paths in the coating, that can fill with the environmental fluid and allow the fluid to enter the core and dissolve the drug, and modify the release characteristics of the formulation. The pore formers can be inorganic or organic, and include materials that can be dissolved, extracted or leached from the coating in the environment of use.

The term “pharmaceutically acceptable” as used herein refers to compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The term “subject” or “patient” as used herein means all members of the animal kingdom, in particular, humans.

The term “effective amount” as used herein means a “pharmaceutically effective amount”. A “pharmaceutically effective amount” is the amount or quantity of a drug which is sufficient to elicit an appreciable biological response when administered to a patient, or patient in need thereof. It will be appreciated that the precise therapeutic dose will depend on the age and condition of the patient, or patient in need thereof, and the nature of the condition to be treated and will be at the ultimate discretion of the attendant physician.

The compositions and methods of the present invention can be used for at least one of the following: to treat a disease, to decrease or alleviate one or more symptoms of a disease, to diminish the extent of a disease, to stabilize (i.e., prevent worsening) of a disease, to slow the spread of a disease, to prevent a disease, to prevent one or more symptoms of a disease, to cause a disease to go into remission, either partially or fully, and to prolong survival as compared to expected survival if not receiving treatment, in a patient or patient in need thereof.

“Disease” as used herein refers to an impairment of health or a condition of abnormal functioning in the body or mind of a subject. Non limiting examples of a disease include:

AIDS, allergies, Alzheimer's disease, anxiety disorders, arthritis, asthma, astigmatism autoimmune diseases, benign prostate hyperplasia (BPH), bipolar disorder (manic-depressive), brain cancer, breast cancer, cancer, candidiasis, cataracts, celiac disease, cervical cancer, chicken pox, chlamydia, chronic fatigue syndrome (CFS), chronic illness, cluster headache, cold sores, colon cancer, constipation, common cold, chronic obstructive pulmonary disease (COPD), cough, Crohn's disease, cystic fibrosis, dementia, diabetes, diarrhea, depression, eczema, endometriosis, eye disorders, fibroids, fibromyalgia, flu (influenza), food poisoning, Gallstones, genital herpes, gonorrhea, Graves' disease, Hashimoto's thyroiditis, hay fever, headache, heart disease, hemochromatosis, hepatitis, herpes, high cholesterol, HIV, Hodgkin's disease, HPV (human papilloma virus), hypertension, impotence, insomnia, irritable bowel syndrome, jaundice, kidney disease, lactose intolerance, leukemia, liver cancer, liver disease, lung cancer, lupus, Lyme disease, lymphoma, meningitis, meningococcal disease, menopause, mental illness, myopia (short-sightedness), migraine, multiple sclerosis, muscular dystrophy, narcolepsy, Non-Hodgkin's lymphoma, obesity, osteoporosis, otitis media (middle ear infection), ovarian cancer, overweight, pain, Parkinson's disease, pelvic inflammatory disease, pertussis, pregnancy, premenstrual syndrome (PMS), prostate cancer, prostate disorders, Raynaud's Phenomenon, restless leg syndrome, SARS, sexually transmitted diseases, sleep disorders, smoking, stroke, thrush, thyroid disorders and whooping cough.

The term external environment of use refers to an environment that drug can be released into from an embodiment of the present invention. Non-limiting examples of an external environment of use include a solvent, a solution, the oral cavity, the interior of the stomach, preferably the interior of a human stomach, the interior of a small intestine, preferably the interior of a human small intestine, including the duodenum, the jejunum, and the ileum, the interior of a large intestine, preferably the interior of a human large intestine, including the ascending colon, the traverse colon, the descending colon, the sigmoid colon, the rectum, and the anal canal, and any combination thereof.

Further non-limiting examples of an external environment of use can include a dissolution medium,

wherein the temperature of the dissolution medium is preferably 37° C.+/−0.5° C., and can range, for example, from greater than about 0° C. to about 10° C., including all values, ranges, and subranges therein,
wherein the volume of the dissolution medium is preferably selected from the group consisting of 500 ml and 900 ml, and can range, for example from 1 ml to 10,000 litres, including all values, ranges, and subranges therein,
wherein the dissolution medium is preferably selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl solution containing added sodium chloride in an amount of 14 g/litre of the solution,
wherein the dissolution medium is preferably stirred by a paddle, preferably a USP type II paddle, at preferably 50 rotations per minute or 100 rotations per minute, wherein the stirring rate by the paddle can range, for example, from about greater than 0 rotations per minute to about 5000 rotations per minute, including all values, ranges, and subranges therein, and
wherein the pressure of the atmosphere on the dissolution medium is preferably 1 atmosphere, and can range, for example, from 0.5 atmospheres to 10 atmospheres, including all values, ranges, and subranges therein.

The pH of the dissolution medium, can, for example range from about −1 to about 14, including all values, ranges, and subranges therein.

“Unfavorable interaction” as used herein refers to any interaction between drug and pharmaceutical excipient (e.g. osmotic agent and/or modified-release polymer) that results in a decrease in drug product quality and/or decrease in release of drug from the composition. A non-limiting example of an unfavorable interaction includes the interaction between a drug and a hygroscopic osmotic agent located in the same compartment, wherein the drug is degraded by water that is drawn in by the hygroscopic osmotic agent.

“Weight gain” as used herein refers to the increase in weight of the core after being coated with at least one coating, over the weight of the core before coating.

The term “increased stability”, “greater stability” or “enhanced stability” as used herein are used interchangeably in this application, and mean that the composition and/or the drug contained in the composition, shows at least not more degradation, and in certain embodiments shows less degradation, than an otherwise similar or identical composition without an osmotic subcoat, when exposed to similar or identical conditions. Degradation can be determined, for example, by the difference in dissolution profiles (i.e. difference in rate and/or extent of drug release into the dissolution medium), or any measurable difference in the retention of drug potency, after a certain time period of storage under similar or identical conditions (e.g. 1 day, one week, 1 month, or one year storage at 25° C./60% RH).

The term “increased release” as used herein means that the rate and/or extent of drug release into the dissolution medium by a composition of the present invention containing an osmotic subcoat, is greater than the rate and/or extent of drug release of an otherwise similar or identical composition that does not contain an osmotic subcoat, under similar or identical conditions (e.g. similar or identical dissolution media).

The term “substantially full release” as used herein refers to the extent of drug released into the dissolution medium whereby at least about 90% of the total amount of drug is released during the dissolution period, or at least about 91%, or at least about 92%, or at least about 93%, or at least about 94%, or at least about 95%, or at least about 96%, or at least about 97%, or at least about 98%, or at least about 99%, or at least about 100%, or at least about 90% to about 100% of the drug is released during the dissolution period.

The term “dissolution profile” or “release profile” as used herein are used interchangeably in this application, and means a quality control test conducted according to instructions found in the United States Pharmacopoeia (“USP”), i.e. using a USP apparatus design with a dissolution medium as found in the USP. Dissolution tests in-vitro measure the rate and extent of dissolution of the drug in an aqueous dissolution medium. The dissolution rate or in-vitro release rates of drug from the modified release dosage forms of the present invention can be measured using one of many USP apparatus designs and dissolution media; non-limiting examples of which include a USP Type 1 apparatus design or USP Type 2 apparatus design, with a dissolution medium selected from water; 0.1N HCl; 0.1N HCl with added Sodium Chloride (e.g. 15.7 g NaCl/Litre); 0.1N HCl with added 0.1% Cetrimide; USP Buffer pH 1.5; Acetate Buffer pH 4.5; Phosphate Buffer pH 6.5; Phosphate Buffer pH 6.8; and Phosphate Buffer pH 7.4.

The term “steady state” as used herein means that the blood plasma concentration curve for a given drug does not substantially fluctuate after repeated doses to dose of the formulation.

The term “dose dumping” as used herein in respect of “alcohol induced dose dumping” or “food induced dose dumping” is defined to mean the unintended rapid release of a drug from a modified release dosage form. The term “alcohol induced dose dumping” as used herein is defined to mean the unintended rapid release of a drug under certain solvent conditions (e.g. dissolution medium containing about 40% ethanol, or about 39% ethanol, or about 38% ethanol, or about 37% ethanol, or about 41% ethanol, or about 42% ethanol, or about 43% ethanol). In certain embodiments “alcohol induced dose dumping” is the rapid release of a drug from the modified release dosage form over a period of about 2 hours when dissolution is tested in 900 ml of Alcohol USP comprising dissolution media using USP Apparatus 1 at 75 rpm at 37° C. In certain embodiments the term “Alcohol USP comprising dissolution media” means any dissolution media comprising from about 5 to about 40% (v/v) of Alcohol USP, or from about 10% to about 40%, or from about 15% to about 40%, or from about 20% to about 40%, or from about 25% to about 40%, or from about 30% to about 40%, or from about 35% to about 40% (v/v) of Alcohol USP, including all values, ranges, and subranges therebetween.

“AUC” as used herein means area under the plasma concentration-time curve, as calculated by the trapezoidal rule over about a 24-hour interval; and signifies the extent of the absorption of a drug.

“Cₘₐₓ” as used herein means the highest plasma concentration of the drug attained within the dosing interval, e.g., about 24 hours.

“Cₘᵢₙ” as used herein means the minimum plasma concentration of the drug attained within the dosing interval, e.g., about 24 hours.

“C_{avg}” as used herein means the plasma concentration of the drug within the dosing interval, e.g., about 24-hours, and is calculated as AUC/dosing interval.

“Tₘₐₓ” as used herein means the time period which elapses after administration of the dosage form at which the plasma concentration of the drug attains the highest plasma concentration of drug attained within the dosing interval (e.g., about 24 hours).

“Bioequivalence” as used herein is defined as there being a 90% or greater probability that the bioavailability (AUC) of the drug as determined by standard methods is 80% to 125% of the second orally administrable dosage form comprising the same dose of the drug and that there is 90% or greater probability that the maximum blood plasma concentration (Cₘₐₓ) of the drug as measured by standard methods is 80% to 125% of the second orally administrable dosage form. For example, the reader is referred to the final version of the guidance approved by the US Food and Drug Administration at the time of filing of this patent application i.e., the March 2003 Guidance for Industry Bioavailability and Bioequivalence Studies for Orally Administered Drug Products General Considerations, U.S. Department of Health and Human Services, Food and Drug Administration, Center for Drug Evaluation and Research (CDER), for a detailed discussion on bioequivalence.

The term “first administration” as used herein refers to the first single dose of the composition administered to a patient, or patient in need thereof, or the first dose administered to a patient, or patient in need thereof, after a suitable washout period.

The term “medicament” as used herein refers to all possible oral dosage forms, including but not limited to, all modified release dosage forms, osmosis controlled release systems, erosion controlled release systems, dissolution controlled release systems, diffusion controlled release systems, enteric coated tablets, single and double coated tablets, capsules, minitablets, caplets, coated beads, granules, spheroids, pellets, microparticles and suspensions.

The terms “free of”, “not include”, “without”, or “exclude” as used herein are used interchangeably and mean “lacking an effective amount of”.

The term “a” or “an” as used herein means “one” or “one or more”.

The numerical parameters set forth in the following specification and attached claims that are modified by the term “about”, are approximations that can vary depending upon the technological properties of the particular case. For example, the term “about” can mean within an acceptable range for the particular value as determined by one of ordinary skill in the art, which will depend in part on how the value is measured or determined, e.g., the limitations of the measurement system. At the very least, and not as an attempt to limit the application of the doctrine of equivalents to the scope of the claims, each numerical parameter modified by the term “about” should at least be construed in light of the number of reported significant digits and by applying ordinary rounding techniques. The terms “about” and “approximately” as used herein are used interchangeably.

Other terms are defined as they appear in the following description and should be construed in context with which they appear.

The present invention is directed to a modified release osmotic dosage form that can provide modified release of at least one drug. The present invention can be used with compositions having at least one drug, non-limiting examples of which include low-solubility drugs, low-dose drugs, high-solubility drugs, high-dose drugs, normal-solubility drugs, normal-dose drugs, and/or any mixtures thereof. The osmotic dosage form comprises at least one means for providing increased release of the at least one drug, said means including at least one osmotic subcoat. In at least one embodiment, the osmotic dosage form comprises at least one means for providing full release of the at least one drug, said means including at least one osmotic subcoat.

In at least one embodiment the osmotic dosage form provides a sustained release of at least one drug. In at least one embodiment the osmotic dosage form provides an extended release of at least one drug. In at least one embodiment the osmotic dosage form provides a delayed release of at least one drug, wherein the release profile includes a predetermined lag time. In at least one embodiment the osmotic dosage form provides a delayed release of at least one drug, wherein the predetermined lag time is substantially independent of the pH of the dissolution medium. In at least one embodiment the osmotic dosage form provides a release profile that does not include a lag time.

In certain embodiments, the osmotic dosage form provides increased release of the at least one drug without requiring the use of a pore-former in the modified release overcoat. In at least one embodiment the osmotic dosage form provides increased release of the at least one drug without a pore-former in the modified release overcoat. In at least one embodiment the osmotic dosage form provides full release of the at least one drug without a pore-former in the modified release overcoat. In at least one embodiment the osmotic dosage form provides full release of the at least one drug in from about 16 hours to about 24 hours, without a pore-former in the modified release overcoat.

In certain embodiments, the osmotic dosage form provides the skilled artisan with the flexibility to control the release profile by manipulating the level of the osmotic agent and/or the level of the osmotic deposition vehicle in the formulation, without requiring the use of a sealcoat around the core. In at least one embodiment, the osmotic dosage form provides the skilled artisan with the flexibility to control the release profile without a sealcoat surrounding the core.

In certain embodiments the osmotic delivery system is a multiparticulate system comprising a plurality of microparticle cores that are each surrounded by an osmotic subcoat, which in turn is surrounded by a functional coat (e.g. modified-release overcoat). In at least one embodiment the microparticle cores include at least one drug. In at least one embodiment the microparticle cores can be further surrounded by at least one non-functional coat. In at least one embodiment an additional overcoat containing at least one drug can surround the modified-release overcoat of each microparticle. In at least one embodiment the coated microparticles (e.g. coated with at least one osmotic subcoat and at least one modified release overcoat) can be compressed into a tablet using suitable excipients. In at least one embodiment an additional overcoat containing at least one drug can surround the tablet. In at least one embodiment the coated microparticles (e.g. coated with at least one osmotic subcoat and at least one modified release overcoat) can be filled into a capsule. In at least one embodiment an additional overcoat containing at least one drug can surround the capsule.

In certain embodiments the osmotic delivery system is a multiparticulate system comprising a plurality of microparticle cores that are each surrounded by an osmotic subcoat. In at least one embodiment the microparticle cores include at least one drug. In at least one embodiment the microparticle cores can be surrounded by at least one functional coat and/or non-functional coat. In at least one embodiment an additional overcoat containing at least one drug can surround the osmotic subcoat of each microparticle. In at least one embodiment the coated microparticles (e.g. coated with at least one osmotic subcoat) can be compressed into a tablet using suitable excipients; wherein such tablet can be surrounded by a modified release overcoat. In at least one embodiment an additional overcoat containing at least one drug can surround the modified release overcoat of the tablet. In at least one embodiment the coated microparticles (e.g. coated with at least an osmotic subcoat) can be filled into a capsule; wherein such capsule can be surrounded by a modified release overcoat. [e.g. L. A. Felton and J. W. McGinity, Enteric Coating of Soft Gelatin Capsules, Drug Development and Technology, 3 (6), 34-39, 2003.] In at least one embodiment an additional overcoat containing at least one drug can surround the modified release overcoat of the capsule.

In certain embodiments the osmotic delivery system is a tablet comprising a plurality of microparticle cores. In such embodiments the microparticle cores are compressed into a tablet using suitable excipients; wherein such tablet is surrounded by an osmotic subcoat, which in turn is surrounded by a modified release overcoat. In at least one embodiment the microparticle cores can be surrounded by at least one functional coat and/or non-functional coat. In at least one embodiment the microparticle cores include at least one drug. In at least one embodiment an additional overcoat containing at least one drug can surround the modified release overcoat of the tablet.

In certain embodiments the osmotic delivery system is a capsule comprising a plurality of microparticle cores. In such embodiments the microparticle cores are filled into a capsule; wherein such capsule is surrounded by an osmotic subcoat, which in turn is surrounded by a modified release overcoat. In at least one embodiment the microparticle cores can be surrounded by at least one functional coat and/or non-functional coat. In at least one embodiment the microparticle cores include at least one drug. In at least one embodiment an additional overcoat containing at least one drug can surround the modified release overcoat of the capsule.

In certain embodiments the osmotic delivery system comprises a matrix core that is surrounded by an osmotic subcoat. In at least one embodiment the matrix core is a modified release matrix core that includes at least one drug, which cores can be surrounded by an osmotic subcoat, which in turn can be surrounded by at least one functional coat and/or non-functional coat. In at least one embodiment the matrix core is an immediate release matrix core that includes at least one drug, which cores can be surrounded by an osmotic subcoat, which in turn can be surrounded by at least one functional coat (e.g. modified release overcoat) and/or non-functional coat. In at least one embodiment an additional overcoat containing at least one drug can surround the modified-release overcoat of the matrix core.

The osmotic subcoat includes an osmotic deposition vehicle and an osmotic agent in an amount sufficient to achieve an osmotic effect across one or more outer coating(s) (e.g. a modified-release overcoat and/or an optional additional overcoat). The modified-release overcoat includes a modified release polymer, and in certain embodiments, does not include a hydrophilic pore former. The coated microparticles of certain embodiments can be filled into capsules or can be compressed into tablets using suitable excipients. In certain embodiments the dissolution profile of the multiparticulates and the effect of the osmotic agent are substantially unaffected by the compression of the multiparticulates into a tablet.

In addition to tablets and capsules, the skilled person will appreciate that the present invention also encompasses other orally and non-orally administerable medicaments such as microparticles, suppositories, sachets, troches, and lozenges, as well as liquid suspensions and elixirs. The present invention can be administered via any route known for administration. Non-limiting routes of administration include administering orally, intra-nasally, rectally, intra-muscularly, intra-venously, sub-cutaneously, trans-dermally, intra-occularly, topically, via inhalation into the lungs, sub-lingually, intra-vaginally, or through the ear canal.

The multiparticulate osmotic delivery system of certain embodiments of the present invention can utilize both diffusion and osmosis to control drug release, and can be incorporated into a modified-release dosage form. The osmotic subcoat and the modified-release polymer overcoat are versatile in that they can be used to coat a variety of drug cores, and can each be manipulated to obtain various desired drug release profiles. Certain embodiments of the present invention provide for increased release of at least one drug from the dosage form without requiring the use of pore forming additives in the functional coat (e.g. modified-release polymer overcoat). In at least one embodiment the composition provides increased release of the drug(s) without a pore former in the modified release overcoat. In at least one embodiment the modified release overcoat is free of pore formers. In certain embodiments, increased release of the drug is achieved in about 24 hours or less without the use of pore forming additives in the modified-release polymer overcoat. In at least one embodiment, substantially full release of the drug can be achieved in about 24 hours or less without the use of pore forming additives in the modified-release polymer overcoat. For example, in at least one embodiment, at least about 90%, or about 91%, or about 92%, or about 93%, or about 94%, or about 95%, or about 96%, or about 97%, or about 98%, or about 99%, or about 100%, or about 90% to about 100%, including all values, ranges and subranges of the total amount of the drug (e.g. rivastigmine) is released within about 24 hours without the use of pore forming additives. In certain embodiments, increased release of a low-dose drug is achieved in about 24 hours or less without the use of a pore forming additive in a functional coat (e.g. the modified-release overcoat) or non-functional coat (e.g. taste-masking coat). In at least one embodiment, substantially full release of a low-dose drug, for example, at least about 90%, or about 91%, or about 92%, or about 93%, or about 94%, or about 95%, or about 96%, or about 97%, or about 98%, or about 99%, or about 100%, or about 90% to about 100% release, including all values, ranges and subranges therebetween, of the low-dose drug release can be achieved in about 24 hours or less without the use of pore forming additives in the modified-release polymer overcoat. In certain embodiments, increased release of a low-solubility drug is achieved in about 24 hours or less without the use of pore forming additives in the modified-release polymer overcoat. In at least one embodiment, substantially full release of a low-solubility drug, for example, at least about 90%, or about 91%, or about 92%, or about 93%, or about 94%, or about 95%, or about 96%, or about 97%, or about 98%, or about 99%, or about 100% release, or about 90% to about 100% release, including all values, ranges and subranges therebetween, of the low-solubility drug can be achieved in about 24 hours or less without the use of pore forming additives in the modified-release polymer overcoat. In certain embodiments, increased release of a high-dose drug is achieved in about 24 hours or less without the use of pore forming additives in the modified-release polymer overcoat. In at least one embodiment, substantially full release of a high-dose drug, for example, in at least one embodiment, at least about 90%, or about 91%, or about 92%, or about 93%, or about 94%, or about 95%, or about 96%, or about 97%, or about 98%, or about 99%, or about 100% release, or about 90% to about 100% release, including all values, ranges and subranges therebetween, of the high-dose drug, can be achieved in about 24 hours or less without the use of pore forming additives in the modified-release polymer overcoat. In certain embodiments, increased release of a high-solubility drug is achieved in about 24 hours or less without the use of pore forming additives in the modified-release polymer overcoat. In at least one embodiment, substantially full release of a high-solubility drug, for example, at least about 90%, or about 91%, or about 92%, or about 93%, or about 94%, or about 95%, or about 96%, or about 97%, or about 98%, or about 99%, or about 100% release, or about 90% to about 100% release, including all values, ranges and subranges therebetween, of the high-solubility drug, can be achieved in about 24 hours or less without the use of pore forming additives in the modified-release polymer overcoat. In certain embodiments, increased release of a normal-dose drug is achieved in about 24 hours or less without the use of pore forming additives in the modified-release polymer overcoat. In at least one embodiment, substantially full release of a normal-dose drug, for example, at least about 90%, or about 91%, or about 92%, or about 93%, or about 94%, or about 95%, or about 96%, or about 97%, or about 98%, or about 99%, or about 100% release, or about 90% to about 100% release, including all values, ranges and subranges therebetween, of the normal-dose drug, can be achieved in about 24 hours or less without the use of pore forming additives in the modified-release polymer overcoat. In certain embodiments, increased release of a normal-solubility drug is achieved in about 24 hours or less without the use of pore forming additives in the modified-release polymer overcoat. In at least one embodiment, substantially full release of a normal-solubility drug, for example, at least about 90%, or about 91%, or about 92%, or about 93%, or about 94%, or about 95%, or about 96%, or about 97%, or about 98%, or about 99%, or about 100%, or about 90% to about 100%, including all values, ranges and subranges, of the normal-solubility drug, can be achieved in about 24 hours or less without the use of pore forming additives in the modified-release polymer overcoat.

The microparticles of certain embodiments can be coated with one or more functional or non-functional coatings. Non-limiting examples of functional coatings include osmotic subcoats, modified release polymeric coatings (i.e. modified release overcoat), moisture barrier coatings, and enteric polymeric coatings. Non-functional coatings are coatings that do not substantially affect drug release, but which affect other properties such as the chemical, biological or physical stability, the taste, and the physical appearance of the formulation. Non-limiting examples of non-functional coatings include taste-masking coatings.

The modified release overcoat can be used to provide modified-release of the drug(s). For example, in certain embodiments a modified release dosage form is provided with increased release of the drug(s). In certain embodiments a composition is provided with increased release of the drug(s), and exhibits a dissolution profile such that substantially full release of the drug(s) can be achieved in from about 3 hours to at least about 16 hours. In at least one embodiment the composition exhibits a dissolution profile such that substantially full release of the drug(s) is achieved in about 24 hours or less, or from about 24 hours to about 1 hour. Certain embodiments include once-daily modified release dosage forms with substantially full release of the drug being achieved in from about 3 hours to at least about 24 hours. In addition, delayed release dosage forms with a pre-determined lagtime can be made wherein the lagtime is independent of the pH of the dissolution medium. In certain embodiments a delayed release composition is provided with a lagtime of from about 1 hour to at least about 8 hours. In certain embodiments delayed release compositions are provided with a pre-determined lagtime, and increased release of the drug(s). In at least one embodiment a delayed release composition is provided with a pre-determined lag time and substantially full release of the drug(s) being achieved in from about 12 hours to at least about 24 hours.

Certain embodiments exhibit pulsatile release of the drug(s) by providing an additional overcoat to substantially surround the modified-release polymer overcoat. This additional overcoat can be an immediate release overcoat that includes at least one drug and at least one low viscosity hydrophilic polymer. As such, certain of these embodiments provide an immediate release of at least one drug from the additional overcoat in a first phase of drug release, and then a subsequent modified release (e.g. delayed release) of the drug from the core in a second phase of drug release. The combination of immediate release of a drug from the additional overcoat, coupled with subsequent modified release of a drug from the core, constitutes pulsatile release in certain embodiments. In certain embodiments the drug(s) released from the additional overcoat in the first phase of drug release is different from the drug(s) that is released from the core in the second phase of drug release.

In certain embodiments, pulsatile release of the drug(s) can be provided by combining a first group of microparticles each having for example, immediate release properties, with a second group of microparticles each having for example, modified release properties (e.g. each having a modified release overcoat). In certain other embodiments, pulsatile release of the drug(s) is provided by combining a first group of microparticles having a modified release profile, with a second group of microparticles having a modified release profile different from that of the first group of microparticles. For example, certain embodiments can include a first group of microparticles each having a modified release overcoat with an extended release profile, combined with a second group of microparticles each having a different modified release overcoat with a delayed release profile. As such, these embodiments could provide an extended release of the at least one drug from the first group of microparticles in a first phase of drug release, and then a subsequent delayed release of the at least one drug or at least one other drug from the second group of microparticles in a second phase of drug release.

Without wishing to be bound to any particular theory, it is believed that in certain embodiments, when the composition is in the presence of an aqueous medium, the modified release overcoat acts as a permeable membrane and a flux of aqueous medium/solvent (e.g. water) into the composition is established. This in turn dissolves the osmotic agent in the osmotic subcoat and the drug in the core. As the osmotic agent dissolves, it creates an increased osmotic pressure gradient for the transport of drug solution from the core out into the external environment of use. The rate at which water is able to pass in through the modified release overcoat, osmotic subcoat and into the core, and how quickly the drug solution can pass out of the system, govern the rate of release. The rate and/or extent of drug release can be altered, for example, by changing the thickness and composition (e.g. level of osmotic agent) of the osmotic subcoat, and/or the thickness and composition of the modified release overcoat.

The composition of certain embodiments of the present invention provides for the substantial separation of the drug in the core from the osmotic agent in the osmotic subcoat. While not wishing to be bound to any particular theory, it is believed that due to the substantial separation of the drug from the osmotic agent into at least two separate compartments, interactions between the drug and the osmotic agent and/or other components in the coatings of the composition (e.g. osmotic subcoat, modified release overcoat, and/or any additional overcoats) can be reduced, and consequently the stability of the composition can be increased. In at least one embodiment the composition comprising an osmotic subcoat can have increased stability.

The delayed release characteristics of certain embodiments of the invention do not depend upon enteric coatings or other pH-dependent release-modifying coatings. In at least one embodiment a delayed release dosage forms is provided with a pre-determined lagtime that is independent of the pH of the dissolution medium. While not wishing to be bound to any particular theory, it is believed that due to the delay in the release of the drug from modified release compositions of certain embodiments of the present invention, and the slow but gradual rise in the plasma concentrations of the drug after first administration of the composition, the incidence of adverse events seen in individuals administered with the compositions of the invention is less than that of immediate release formulations. By formulating a formulation with a pre-determined lagtime that is independent of the pH of the dissolution medium, it is possible to more easily target a specific segment of the gastro-intestinal tract. For example, in certain embodiments an acid labile drug can be prevented from being released in the stomach, or can be released in the colon (i.e. colonic delivery).

In certain embodiments wherein the drug(s) include pramipexole, a modified release dosage form is provided that includes at least one means for the modified release of pramipexole such that after about 2 hours from about 0% to about 25% by weight of pramipexole can be released, or from about 5% to about 25% by weight of pramipexole can be released, including all values, ranges and subranges therebetween, including, for example, from about 10% to about 25% by weight of pramipexole can be released, or from about 15% to about 25% by weight of parmipexole can be released, or from about 20% to about 25% by weight of pramipexole can be released; after about 4 hours from about 15% to about 70% by weight of pramipexole can be released, including all values, ranges and subranges therebetween, including, for example, from about 20% to about 70% by weight of pramipexole can be released, from about 30% to about 70% by weight of pramipexole can be released, or from about 40% to about 70% by weight of pramipexole can be released, or from about 50% to about 70% by weight of pramipexole can be released, or from about 60% to about 70% by weight of pramipexole can be released; after about 6 hours from about 30% to about 95% by weight of pramipexole can be released, including all values, ranges and subranges therebetween, including, for example, from about 40% to about 95% by weight of pramipexole can be released, or from about 50% to about 95% by weight of pramipexole can be released, or from about 60% to about 95% by weight of pramipexole can be released, or from about 70% to about 95% by weight of parmipexole can be released, or from about 80% to about 95% by weight of parmipexole can be released, or from about 90% to about 95% by weight of pramipexole can be released; and after about 8 hours more than about 50% by weight of pramipexole can be released, for example about 60% by weight, about 70% by weight, about 80% by weight, about 90% by weight, or about 100% by weight of the pramipexole can be released.

In certain embodiments wherein the drug(s) include diltiazem, a modified release dosage form is provided that includes at least one means for the modified release of diltiazem such that after about 2 hours from about 0% to about 30% by weight of diltiazem can be released, including all values, ranges and subranges therebetween, including, for example, from about 10% to about 30% by weight of diltiazem can be released, or from about 20% to about 30% by weight of diltiazem can be released; after about 4 hours from about 50% to about 80% by weight of diltiazem can be released, including all values, ranges and subranges therebetween, including, for example, from about 60% to about 80% by weight of diltiazem can be released, or from about 70% to about 80% by weight of diltiazem can be released; and after about 8 hours more than about 80% by weight of diltiazem can be released, including all values, ranges and subranges therebetween, including, for example about 85% by weight of diltiazem can be released, for example about 90% by weight of diltiazem can be released, or for example about 95% by weight of diltiazem can be released.

In certain embodiments wherein the drug(s) include rivastigmine, a modified release dosage form is provided that includes at least one means for the modified release of rivastigmine such that after about 2 hours from about 0% to about 5% by weight of rivastigmine can be released, or about 1% or about 2% or about 3% or about 4% by weight of rivastigmine can be released; after about 4 hours from about 0% to about 20% by weight of rivastigmine can be released, including all values, ranges and subranges therebetween, including, for example, from about 5% to about 20% by weight of rivastigmine can be released, or from about 10% to about 20% by weight of rivastigmine can be released; after about 6 hours from about 10% to about 60% by weight of rivastigmine can be released, including all values, ranges, and subranges therebetween, including, for example, from about 20% to about 60% by weight of rivastigmine can be released, or from about 30% to about 60% by weight of rivastigmine can be released, or from about 40% to about 60% by weight of rivastigmine can be released, or from about 50% to about 60% by weight of rivastigmine can be released; after about 8 hours from about 30% to about 80% by weight of rivastigmine can be released, including all values, ranges and subranges therebetween, including, for example, from about 40% to about 80% by weight of rivastigmine can be released, or from about 50% to about 80% by weight of rivastigmine can be released, or from about 60% to about 80% by weight of rivastigmine can be released, or from about 70% to about 80% by weight of rivastigmine can be released; after about 10 hours from about 40% to about 95% by weight of rivastigmine can be released, including all values, ranges and subranges therebetween, including, for example, from about 50% to about 95% by weight of rivastigmine can be released, or from about 60% to about 95% by weight of rivastigmine can be released, or from about 70% to about 95% by weight of rivastigmine can be released; and after about 12 hours more than about 50% by weight of rivastigmine can be released, or about 60%, or about 70%, or about 80%, or about 90%, or about 100% by weight of rivastigmine can be released.

In certain embodiments wherein the drug(s) include rivastigmine, a delayed release dosage form is provided that includes at least one means for the delayed release of rivastigmine such that after about 6 hours from about 0% to about 5% by weight, including all values, ranges and subranges therebetween, including for example, about 1% by weight, or about 2% by weight, or about 3% by weight, or about 4% by weight of rivastigmine can be released; after about 8 hours from about 0% to about 10% by weight, including all values, ranges and subranges therebetween, including, for example, about 1% by weight, or about 2% by weight, or about 3% by weight, or about 4% by weight, or about 5% by weight, or about 6% by weight, or about 7% by weight, or about 8% by weight, or about 9% by weight of rivastigmine can be released; after about 12 hours from about 10% to about 40% by weight, including all values, ranges and subranges therebetween, including for example, about 15% by weight, or about 20% by weight, or about 25% by weight, or about 30% by weight, or about 35% by weight of rivastigmine can be released; after about 18 hours from about 40% to about 80% by weight, including all values, ranges and subranges therebetween, including for example, about 50% by weight, or about 60% by weight, or about 70% by weight of rivastigmine can be released; and after about 24 hours more than about 80% by weight, or about 90% by weight, or about 95% by weight, or about 100% by weight of rivastigmine can be released.

In certain embodiments wherein the drug(s) includes dexmethylphenidate, a modified release dosage form is provided that includes at least one means for the modified release of dexmethylphenidate such that after about 2 hours from about 0% to about 5% by weight, including all values, ranges and subranges therebetween, including for example, about 1% by weight, or about 2% by weight, or about 3% by weight, or about 4% by weight of dexmethylphenidate can be released; after about 4 hours from about 0% to about 20% by weight, including all values, ranges and subranges therebetween, including for example, about 5% by weight, or about 10% by weight, or about 15% by weight of dexmethylphenidate can be released; after about 6 hours from about 10% to about 60% by weight, including all values, ranges and subranges therebetween, including, for example, about 20% by weight, or about 30% by weight, or about 40% by weight, or about 50% by weight of dexmethylphenidate can be released; after about 8 hours from about 30% to about 80% by weight, including all values, ranges and subranges therebetween, including for example, about 40% by weight, or about 50% by weight, or about 60% by weight, or about 70% by weight of dexmethylphenidate can be released; after about 10 hours from about 40% to about 95% by weight, including all values, ranges and subranges therebetween, including for example, about 50% by weight, or about 60% by weight, or about 70% by weight, or about 80% by weight, or about 90% by weight of dexmethylphenidate can be released; and after about 12 hours more than about 50% by weight, or about 60% by weight, or about 70% by weight, or about 80% by weight, or about 90% by weight, or about 100% by weight of dexmethylphenidate can be released.

In certain embodiments wherein the drug(s) includes dexmethylphenidate, a delayed release dosage form is provided that includes at least one means for the delayed release of dexmethylphenidate such that after about 6 hours from about 0% to about 5% by weight, including all values, ranges and subranges therebetween, including for example, about 1% by weight, or about 2% by weight, or about 3% by weight, or about 4% by weight of dexmethylphenidate can be released; after about 8 hours from about 0% to about 10% by weight, including all values, ranges and subranges therebetween, including for example, about 1% by weight, or about 2% by weight, or about 3% by weight, or about 4% by weight, or about 5% by weight, or about 6% by weight, or about 7% by weight, or about 8% by weight, or about 9% by weight of dexmethylphenidate can be released; after about 12 hours from about 10% to about 40% by weight, including all values, ranges and subranges therebetween, including for example, about 20% by weight, or about 30% by weight of dexmethylphenidate can be released; after about 18 hours from about 40% to about 80% by weight, or about 50% by weight, or about 60% by weight, or about 70% by weight of dexmethylphenidate can be released; and after about 24 hours more than about 80% by weight, or about 90% by weight, or about 100% by weight of dexmethylphenidate can be released.

The term “microparticles” as used herein is interchangeable with the terms “microspheres”, “spherical particles”, “microcapsules”, “particles”, “multiparticulates”, “granules”, “spheroids”, beads” and “pellets”.

The microparticles of the present invention include an effective amount of at least one drug and at least one pharmaceutically acceptable excipient. The microparticles can be contained within a capsule, or can be compressed into a matrix or tablet, that upon ingestion dissolves into multiple units (e.g. pellets), wherein the sub-units or pellets possess the desired modified release properties of the dosage form. The microparticles or the multiple unit dosage forms can include one or more functional coatings and/or non-functional coatings. Non-limiting examples of such coatings include polymeric controlled release coatings, delayed release coatings, enteric coatings, immediate release coatings, taste-masking coatings, extended release coatings, and seal or barrier coatings.

Drug(s) include those that are sparingly soluble and whose dissolution and release properties in-vivo can be enhanced by an osmotic system (i.e. low-solubility drugs). Drug(s) also include those that are high in potency, or dosed in low concentrations (i.e. low-dose drugs). In addition, a wide range of other types of drug(s) can be used in the present invention, including high-solubility drugs, normal-solubility drugs, high-dose drugs, normal-dose drugs, and mixtures thereof. Non-limiting examples of drugs include bupropion, carvedilol, citalopram, diltiazem, fluoxetine, metoprolol, pramipexole, quetiapine, ramipril, rivastigmine, rosiglitazone, sumatriptan, topiramate, tramadol, venlafaxine, zolpidem, memantine, meloxicam, dexmethylphenidate, their pharmaceutically acceptable salts, and mixtures thereof.

The term “low-solubility drug,” refers to drug(s) wherein at least about 30 parts of water is used to dissolve one part of drug. For example, low-solubility drugs can include practically insoluble drugs, meaning that at least about 10,000 parts of water is used to dissolve one part of drug; very slightly soluble drugs, meaning that from about 1,000 parts to about 10,000 parts of water is used to dissolve one part of drug; slightly soluble drugs, meaning that from about 100 parts to about 1,000 parts of water is used to dissolve one part of drug; or sparingly soluble drugs, meaning that from about 30 parts to about 100 parts of water is used to dissolve one part of drug.

The term “low-dose drug” refers to drugs that are dosed at about 20 mg or less per dosage form.

In certain embodiments the composition provides increased release of a low-dose drug without the use of a pore forming additive in the modified-release polymer overcoat. In at least one embodiment the composition provides substantially full release of a low-dose drug without the use of a pore forming additive in the modified-release polymer overcoat. In certain embodiments the composition provides increased release of a low-solubility drug without the use of a pore forming additive in the modified-release polymer overcoat. In at least one embodiment the composition provides substantially full release of a low-solubility drug without the use of a pore forming additive in the modified-release polymer overcoat.

Drug(s) that can be used herein are selected from a large group of therapeutic agents. Respective classes include but are not limited to those in the following therapeutic categories: ace-inhibitors; alkaloids; anabolic agents; analgesics; antacids; anti-allergy agents; anti-Alzheimer's Disease agents; anti-anginal drugs; antianxiety agents; anti-arrhythmia agents; antiasthmatics; antibacterial and antifungal agents; antibiotics; anticholesterolemics; anticlotting agents; anticonvulsants; anticoagulants; antidepressants; antidiarrheal preparations; anti-emetics; antihistamines; antihyperglycemic agents; antihypertensives; anti-impotence agents; anti-infectives; anti-inflammatories; antilipid agents; antimanics; anti-migraine agents; antinauseants; antineoplastics; antiobesity agents; antiparasitics; anti-Parkinsonism agents; antipsychotics; antipyretics; antispasmodics; antistroke agents; antithrombotics; antithyroid preparations; antitumor agents; antitussives; antiulcer agents; anti-uricemic agents; antiviral agents; anxiolytic agents; appetite stimulants; appetite suppressants; autoimmune disorders agents; barbiturates; beta-blocking agents; blood glucose-lowering agents; bronchodilators; cardiovascular agents; cerebral dilators; chelating agents; cholecystekinin antagonists; chemotherapeutic agents; cholesterol-reducing agents; cognition activators; cognitive enhancers; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotic agents; hypoglycemic agents; ion-exchange resins; laxatives; migraine treatments; mineral supplements; mucolytics, narcotics; neuroleptics; neuromuscular drugs; non-steroidal anti-inflammatories (NSAIDs); nutritional additives; peripheral vasodilators; polypeptides; prostaglandins; psychotropics; renin inhibitors; respiratory stimulants; sedatives; steroids; stimulants; sympatholytics; thyroid preparations; tranquilizers; uterine relaxants; vaginal preparations; vasoconstrictors; vasodilators; vertigo agents; vitamins; wound healing agents; and others.

Drugs which can be used in the present invention include but are not limited to: acetazolamide; acetaminophen; acetic acid; acetohexamide; acetylsalicylic acid, including its buffered forms; acrivastine; acyclovir; albuterol and its sulfate; alcohol; alfaxalone; alkaline phosphatase; allantoin; aloe; alprostadil; aluminum acetate, carbonate, chlorohydrate and hydroxide; alprozolam; amino acids; aminobenzoic acid; amlodipine besylate; amoxicillin; ampicillin; amsacrine; amsalog; anethole; apomorphine; ascorbic acid; aspartame; aspirin; astemizole; atenolol; atorvastatin calcium; azatidine and its maleate; azithromycin; bacitracin; balsam peru; BCNU (carmustine); becampicillin hydrochloride; beclomethasone diproprionate; benzalkonium chloride; benzocaine; benzoic acid; benzophenones; benzoyl peroxide; benzquinamide and its hydrochloride; betamethasone; bethanechol; biotin; bisacodyl; bismuth subsalicylate; bomyl acetate; bromopheniramine and its maleate; bupropion; buspirone; caffeine; calamine; calcium carbonate, casinate and hydroxide; camphor; captopril; carbenicillin indanyl sodium; carvedilol; cascara sagrada; castor oil; cefaclor; cefadroxil; celicoxib; cephalexin; centrizine and its hydrochloride; cetirizine; cetyl alcohol; cetylpyridinium chloride; chelated minerals; chlorambucil; chloramphenicol; chlorcyclizine hydrochloride; chlordiazepoxide; chlorhexidine gluconate; chloroxylenol; chloropentostatin; chlorpheniramine and its maleates and tannates; chlorpromazine; chlorpropamide; chlorthalidone; chlorzolamide; cholestyramine resin; choline bitartrate; chondrogenic stimulating protein; cimetidine and its hydrochloride; cinnamedrine hydrochloride; cinnarizine; cisapride; citalopram; citric acid; clarithromycin; clemastine and its flumarate; clonidine and its hydrochloride salt; clorfibrate; cocoa butter; cod liver oil; codeine and its fumarate and phosphate; cortisone acetate; cotrimoxazole; ciprofloxacin HCl; cyanocobalamin; cyclizine hydrochloride; cyproheptadine and its hydrochloride; dexmethylphenidate; danthron; dexbromopheniramine maleate; dextromethorphan and its hydrohalides; diazepam; dibucaine; dichloralphenazone; diclofen and its alkali metal sales; diclofenac sodium; dicumarol; digitoxin; digoxin; dihydroergotamine and its hydrogenates/mesylates; diltiazem; dimenhydrinate; dimethicone; dioxybenzone; diphenhydramine and its citrate; diphenhydramine and its hydrochloride; divalproex and its alkali metal salts; docusate calcium, potassium, and sodium; donepezil; doxazosin; doxepin and its hydrochloride salt; doxycycline hydrate; doxylamine succinate; dronabinol; echinomycin; econazole; efaroxan; enalapril; enalaprilic acid; enoxacin; ephedrine; epinephrine bitartrate; ergotamine and its tartrate; erythromycin; erythropoietin; estropipate; ethinyl estradiol; etomidate; eucalyptol; famotidine; fenoprofen and its metal salts; ferrous fumarate, gluconate and sulfate; fluconazole; fluoxetine; fluoxymesterone; folic acid; fosphenytoin; 5-fluorouracil (5-FU); fluoxetine and its hydrochloride; flurbiprofen; fluspirilene; furosemide; gabapentan; gentamicin; gemfibrozil; glipizide; glycerine; glyceryl stearate; granisetron and its hydrochloride; griseofulvin; guafenesin; hexylresorcinol; hydrochlorothiazide; hydrocodone and its tartrates; hydrocortisone and its acetate; 8-hydroxyquinoline sulfate; hydroxyzine and its pamoate and hydrochloride salts; ibuprofen; indomethacin; inositol; insulin; iodine; ipecac; iron; iroxicam; isosorbide and its monoand dinitrates; isoxicam; kaolin; ketamine; ketanserin; ketoprofen; lactic acid; lanolin; L-DOPA; lecithin; leuprolide acetate; levocabastine; lidocaine and its hydrochloride salt; lifinopril; liotrix; lisinopril; lomustine; loperamide; loratadine; lovastatin; magnesium carbonate, hydroxide, salicylate, and trisilicate; meclizine and its hydrochloride; mefenamic acid; meclofenamic acid; meclofenamate sodium; medroxyprogesterone acetate; meloxicam; memantine; methenamine mandelate; menthol; meperidine hydrochloride; metaproterenol sulfate; methanstenolone; methscopolamine and its nitrates; methsergide and its maleate; methyl nicotinate; methyl salicylate; methyl cellulose; methsuximide; 17-methyltestosterone; metoclopramide and its halides/hydrates; metronidazole and its hydrochloride; metoprolol; metoprotol tartrate; mianserin; miconazole nitrate; mineral oil; minocycline; minoxidil; mioflazine; morphine; nadolol; naproxen and its alkali metal sodium salts; nifedipine; neomycin sulfate; niacin; niacinamide; nicotine; nicotinamide; nimesulide; nitroglycerine; nonoxynol-9; norethindrone and its acetate; nystatin; octoxynol; octoxynol-9; octyl dimethyl PABA; octyl methoxycinnamate; omega-3 polyunsaturated fatty acids; omeprazole; ondansetron and its hydrochloride; oxfendazole; oxolinic acid; oxybenzone; oxtriphylline; para-aminobenzoic acid (PABA); padimate-O; paramethadione; paroxetine; penfluridole; penicillin G; pentastatin; peppermint oil; pentaerythritol tetranitrate; pentobarbital sodium; perphenazine; phenelzine sulfate; phenindamine and its tartrate; pheniramine maleate; phenobarbital; phenol; phenolphthalein; phenylephrine and its tannates and hydrochlorides; phenylpropanolamine and its hydrochloride salt; phenyloin; pirmenol; piroxicam and its salts; polymicin B sulfate; potassium chloride and nitrate; pramipexole; pramiracetin; pramoxine and its hydrochloride salt; prazepam; prazosin; prednisolone; procainamide hydrochloride; procaterol; promethazine and its hydrochloride; propoxyphene and its hydrochloride and napsylate; prochlorperazine and its maleate; propanolol and its hydrochloride; promethazine and its hydrochloride; propanolol; prostacyclin; pseudoephedrine and its sulfates and hydrochlorides; pyridoxine; pyrolamine and its hydrochlorides and tannates; quetiapine; quinapril; quinidine gluconate and sulfate; quinestrol; ralitoline; ramipril; ranitadine; resorcinol; retinol; riboflavin; rivastigmine; rosiglitazone; salicylic acid; scopolamine; sertraline; sesame oil; shark liver oil; sildenafil citrate; simethicone; sodium bicarbonate, citrate, and fluoride; sodium monofluorophosphate; spiramycin; spironolactone; sucralfate; sulfanethoxazole; sulfasalazine; sulfur; sulpiride; sumatriptan and its succinate; tacrine and its hydrochloride; terconazole; terfenadine; testosterone; tetracycline; tetracycline hydrochloride; THA; theophylline; thiabendazole; thiethylperazine and its maleate; thioperidone; thiothixene hydrochloride; timolol and its maleate; tolmetin; tolnaftate; topiramate; tramadol; tretinoin; triazolam; trimetrexate; trimazosin; triclosan; trimethobenzamide and its hydrochloride; tripelennamine and its hydrochloride; tripolidine hydrochloride; troleandomycin; tubulazole; undecylenic acid; valdecoxib; vancomycin; venlafaxine; verapamil HCl; vidaribine phosphate; virazole; vitamins A, C, D, B1, B2, B3, B4, B5, B6, B7, B9 and B12, E, and K; witch hazel; xylometazoline hydrochloride; zinc; zinc sulfate; zinc undecylenate; ziprasidone; and zolpidem. The invention also contemplates combinations of the above drugs.

In certain embodiments comprising microparticle dosage forms, the drug(s) can be present in an effective amount from about 0.1% to about 99% by weight of the microparticles. For example, in certain embodiments the drug is present in the microparticles in an amount of from about 0.1% to about 90%, in other embodiments from about 5% to about 90%, in still other embodiments from about 10% to about 80%, and in even still other embodiments from about 25% to about 80% by weight, or from about 10% to about 99% by weight, or from about 20% to about 99% by weight, or from about 30% to about 99% by weight, or from about 40% to about 99% by weight, or from about 50% to about 99% by weight, or from about 60% to about 99% by weight, or from about 70% to about 99% by weight, or from about 80% to about 99% by weight, or from about 90% to about 99% by weight of the microparticle. In certain embodiments wherein the microparticles are manufactured using a spheronization process, the drug can be present in the microparticles in an amount of from about 0.1% to about 60%; in other such embodiments from about 5% to about 50%; and in still other such embodiments from about 10% to about 40% by weight of the microparticle.

The effective amount of drug(s) present is dependent at least in part on the drug(s), the desired modified release profile, and the strength of the desired dosage form. The term “effective amount” as used herein means that a “pharmaceutically effective amount” is contemplated. A “pharmaceutically effective amount” is the amount or quantity of the at least one drug in a dosage form of the invention sufficient to elicit an appreciable clinical or therapeutic response when administered, in single or multiple doses, to a patient, or a patient in need thereof.

In addition to the at least one drug, the microparticles of certain embodiments also include at least one pharmaceutically acceptable excipient. Excipients can be added to facilitate in the preparation, patient acceptability and functioning of the dosage form as a drug delivery system. Non-limiting examples of excipients include spheronization aids, solubility enhancers, disintegrating agents, diluents, lubricants, binders, fillers, glidants, suspending agents, emulsifying agents, anti-foaming agents, flavouring agents, colouring agents, chemical stabilizers, pH modifiers, swelling agents, and mixtures thereof. Depending on the intended main function, the excipients that can be used in formulating the microparticles are subcategorized into different groups. However, certain excipients can affect the properties of a microparticle composition in a series of ways and can thus be described as being multifunctional.

The microparticles of certain embodiments of the present invention can be manufactured using standard techniques known to those of skill in the art. Useful microparticles include drug-containing microparticles.

Drug-containing microparticles can be prepared by a number of different procedures. For example: In a spray drying process, an aqueous solution of core material and hot solution of polymer is atomized into hot air, the water then evaporates, and the dry solid is separated in the form of pellets, for example by air suspension. A spray-drying process can produce hollow pellets when the liquid evaporates at a rate that is faster than the diffusion of the dissolved substances back into the droplet interior, or if due to capillary action the dissolved substance migrates out with the liquid to the droplet surface, leaving behind a void. Another example is a spray congealing process, where a slurry of drug(s) that is insoluble in a molten mass is spray congealed to obtain discrete particles of the insoluble materials coated with the congealed substance. A further example is a fluidized bed based granulation/pelletization process, where a dry drug is suspended in a stream of hot air to form a constantly agitated fluidized bed. An amount of binder or granulating liquid is then introduced in a finely dispersed form to cause pelletization.

Another method of manufacturing drug-containing microparticles of certain embodiments of the present invention is the spheronization process. A non-limiting example is the applicant's proprietary CEFORM™ (Centrifugally Extruded & Formed Microspheres) technology, which is the simultaneous use of flash heat and centrifugal force, using proprietary designed equipment, to convert dry powder systems into microparticles of uniform size and shape. The production of microparticles containing a drug using the CEFORM™ technology is described for example in U.S. Pat. No. 5,683,720. This reference deals with the use of LIQUIFLASH® processing to spheronize compositions containing one or more drugs to form LIQUIFLASH® microparticles.

With the CEFORM™ technology, the processing of the drug-containing microparticles of certain embodiments of the present invention is carried out in a continuous fashion, whereby a pre-blend of drug and excipients is fed into a spinning “microsphere head”, also termed as a “spheronizing head”. The microsphere head, which is a multi-aperture production unit, spins on its axis and is heated by electrical power. The drug and excipient(s) pre-blend is fed into the center of the head with an automated feeder. The material moves, via centrifugal force, to the outer rim where the heaters, located in the rim of the head, heat the material. Microparticles are formed when the molten material exits the head, which are then cooled by convection as they fall to the bottom of the microparticle chamber. The product is then collected and stored in suitable product containers. Careful selection of the types and levels of excipient(s) control microparticle properties such as, for example, sphericity, surface morphology, and dissolution rate. One example of an advantage of such a process is that the microparticles of certain embodiments are produced and collected from a dry feedstock without requiring the use of any solvents. In at least one embodiment the drug-containing microparticles are manufactured using the CEFORM process without the use of any solvents.

There are at least two approaches that can be used to produce drug-containing microparticles using the CEFORM process: (i) the encapsulation approach and (ii) the co-melt approach. In the encapsulation approach, the process is conducted below the melting point of the drug. Therefore, the excipients are designed to melt and entrain the drug particles on passing through the apertures to form microparticles. The resulting microparticles contain the drug in its native state, essentially enveloped by or as an intimate matrix with the resolidified excipients. In the co-melt approach, the process is conducted above the melting point of the drug. In this case, the drug and the excipients melt or become fluid simultaneously upon exposure to the heat. The molten mixture exits the head and forms microparticles, which cool as they fall to the bottom of the collection bin where they are collected.

In at least one embodiment the microparticles are manufactured using the encapsulation approach. In the encapsulation approach the excipient(s) have a lower melting point than the drug with which they will be combined. Therefore the spheronizing process can be performed at lower temperatures than the melting point of the drug. As a result, this can reduce the risk of polymeric interconversion, which can occur when using processing temperatures close to the melting point.

In a prophetic example of certain embodiments of the present invention, the manufacturing process for the drug-containing microparticles can be as follows: A spheronization aid is screened through a 425 μm screen. In at least one embodiment, the spheronization aid is distilled glyceryl monostearate (e.g. DMG-03VF). 50% of the spheronization aid is added to a bowl in a high shear mixer. In at least one embodiment, the bowl is a 6 litre bowl and the high shear mixer is a Diosna P1-6 high speed mixer granulator. The drug is then added to the bowl of the mixer, and then the remainder of the spheronization aid is added. The material is then blended in the mixer for a time from about 1 minute to about 30 minutes; preferably from about 3 minutes to about 10 minutes; and more preferably about 6 minutes. The mixer motor speed is from about 50 rpm to about 2000 rpm; preferably from about 200 rpm to about 500 rpm; and more preferably about 300 rpm. The chopper motor speed is from about 50 rpm to about 2000 rpm; preferably from about 200 rpm to about 500 rpm; and more preferably about 400 rpm. The blended material is then spheronized in a CEFORM™ spheronizing head. The spheronizing head speed is from about 5 Hz to about 60 Hz; preferably from about 10 Hz to about 30 Hz; and more preferably about 15 Hz. In at least one embodiment the CEFORM™ spheronizing head is a 5 inch head. The spheronizing head temperature is maintained at a temperature from about 70° C. to about 130° C.; preferably from about 90° C. to about 110° C.; and more preferably about 100° C. The microparticles obtained from the spinning process are then screened through a screen that is from about 150 μm to about 800 μm, more preferably from about 425 μm to about 800 μm.

For microparticles manufactured using a spheronization process such as the CEFORM™ process, the microparticles include, in addition to the drug, at least one spheronization aid (also known as “spheronization agent”). Spheronization aids can assist the drug-containing mix to form robust durable spherical particles. Some examples of materials useful as spheronization aids include, but are not limited to distilled monoglycerides, glyceryl monostearate, glyceryl behenate, glyceryl dibehenate, glyceryl palmitostearate, hydrogenated oils such as hydrogenated castor oil marketed under the name Cutina HR, fatty acid salts such as magnesium or calcium stearate, polyols such as mannitol, sorbitol, xylitol, stearic acid, palmitic acid, sodium lauryl sulfate, polyoxyethylene ethers, esterified polyoxyethylenes such as PEG-32 distearate, PEG-150 distearate, cetostearyl alcohol, waxes (e.g. carnauba wax, white wax, paraffin wax), wax-like materials, and mixtures thereof. Certain thermo-plastic or thermo-softening polymers can also function as spheronization aids. Some non-limiting examples of such thermo-plastic or thermo-softening polymers include Povidone, cellulose ethers and polyvinylalcohols. Combinations of spheronization aids can be used. In at least one embodiment, the spheronization aid is glyceryl monostearate (e.g. DMG-03VF). The spheronization aid can be present in an amount of from about 0.1% to about 99.9% by weight of the microparticle. For example, in certain embodiments the spheronization aid is present in an amount of from about 5% to about 90%; in other embodiments from about 10% to about 80%; in still other embodiments from about 20% to about 70%; and in even still other embodiments from about 30% to about 60% by weight of the microparticle.

For example, in certain embodiments that include pramipexole (e.g. pramipexole dihydrochloride) as a drug, the spheronization aid(s) can be present in an amount from about 5% to about 99%, in other embodiments from about 40% to about 99%, in still other embodiments from about 90% to about 99%, and in even still other embodiments from about 95% to about 98% by weight of the microparticle. In at least one embodiment the spheronization aid is present in an amount of about 97.5% by weight of the microparticle. In certain embodiments pramipexole can be present in an amount of from about 0.1% to about 80%, in other embodiments from about 0.1% to about 60%, in still other embodiments from about 0.1% to about 10%, and in even still other embodiments from about 1% to about 5% by weight of the microparticle. In at least one embodiment, pramipexole dihydrochloride is present in an amount of about 2.5% by weight of the microparticle. In at least one embodiment, the microparticles include about 50 grams of pramipexole dihydrochloride and about 1950 grams of the spheronization aid(s).

For example, in certain embodiments that include diltiazem (e.g. diltiazem hydrochloride) as a drug, the spheronization aid(s) can be present in an amount of from about 5% to about 99%, in other embodiments from about 10% to about 70%, in still other embodiments from about 30% to about 50%, and in even still other embodiments from about 35% to about 45% by weight of the microparticle. In at least one embodiment the spheronization aid is present in an amount of about 40% by weight of the microparticle. In certain embodiments diltiazem can be present in an amount of from about 1% to about 90%, in other embodiments from about 40% to about 80%, in still other embodiments from about 50% to about 70%, and in even still other embodiments from about 55% to about 65% by weight of the microparticle. In at least one embodiment diltiazem hydrochloride is present in an amount of about 60% by weight of the microparticle. In at least one embodiment, the microparticles include about 1200 grams of diltiazem hydrochloride and about 800 grams of the spheronization aid(s).

For example, in certain embodiments that include rivastigmine (e.g. rivastigmine tartrate) as a drug, the spheronization aid(s) can be present in an amount of from about 5% to about 99%, in other embodiments from about 10% to about 99%, in still other embodiments from about 80% to about 95%, and in even still other embodiments from about 85% to about 95% by weight of the microparticle. In at least one embodiment the spheronization aid is present in an amount of about 90% by weight of the microparticle. In certain embodiments rivastigmine can be present in an amount of from about 0.1% to about 90%, in other embodiments from about 1% to about 40%, in still other embodiments from about 2% to about 20%, and in even still other embodiments from about 5% to about 15% by weight of the microparticle. In at least one embodiment rivastigmine tartrate is present in an amount of about 10% by weight of the microparticle. In at least one embodiment, the microparticles include about 60 grams of rivastigmine tartrate and about 540 grams of the spheronization aid(s).

For example, in certain embodiments that include dexmethylphenidate (e.g. dexmethylphenidate hydrochloride) as a drug, the spheronization aid(s) can be present in an amount of from about 5% to about 99%. In certain embodiments within this example, the spheronization aid(s) can be present in an amount from about 10% to about 99%, in still other embodiments from about 80% to about 95%, and in even still other embodiments from about 85% to about 95% by weight of the microparticle. In at least one embodiment the spheronization aid is present in an amount of about 90% by weight of the microparticle. In certain embodiments dexmethylphenidate can be present in an amount of from about 0.1% to about 90%, in other embodiments from about 1% to about 40%, in still other embodiments from about 2% to about 20%, and in even still other embodiments from about 5% to about 15% by weight of the microparticle. In at least one embodiment dexmethylphenidate is present in an amount of about 10% by weight of the microparticle. In at least one embodiment, the microparticles include about 60 grams of dexmethylphenidate and about 540 grams of the spheronization aid(s).

In certain embodiments, each microparticle can also include at least one solubility enhancer. Certain other embodiments do not include a solubility enhancer. Solubility enhancers can be surfactants. Certain embodiments of the invention include a solubility enhancer that is a hydrophilic surfactant. Hydrophilic surfactants can be used to provide any of several advantageous characteristics to the compositions, including: increased solubility of the drug in the microparticle; improved dissolution of the drug; improved solubilization of the drug upon dissolution; enhanced absorption and/or bioavailability of the drug. The ability of a surfactant to reduce the solid/liquid interfacial tension can permit fluids to wet the solid more effectively and thus aid the penetration of fluids into the drug-surfactant mass to increase the dissolution and absorption of the drug. The hydrophilic surfactant can be a single hydrophilic surfactant or a mixture of hydrophilic surfactants, and can be ionic or non-ionic.

Likewise, various other embodiments of the invention include a lipophilic component, which can be a lipophilic surfactant, including a mixture of lipophilic surfactants, a triglyceride, or a mixture thereof. The lipophilic surfactant can provide any of the advantageous characteristics listed above for hydrophilic surfactants, as well as further enhancing the function of the surfactants. These various embodiments are described in more detail below.

As is well known in the art, the terms “hydrophilic” and “lipophilic” are relative terms. To function as a surfactant, a compound includes polar or charged hydrophilic moieties as well as non-polar hydrophobic (lipophilic) moieties; i.e., a surfactant compound is amphiphilic. An empirical parameter commonly used to characterize the relative hydrophilicity and lipophilicity of non-ionic amphiphilic compounds is the hydrophilic-lipophilic balance (the “HLB” value). Surfactants with lower HLB values are more lipophilic, and can have greater solubility in oils, whereas surfactants with higher HLB values are more hydrophilic, and can have greater solubility in aqueous solutions.

Using HLB values as a rough guide, hydrophilic surfactants can generally be considered to be those compounds having an HLB value greater than 10, for example 11, 12, 13, 14, 15, 16, 17, 18, 19 or 20, as well as anionic, cationic, or zwitterionic compounds for which the HLB scale is not generally applicable. Similarly, lipophilic surfactants can be compounds having an HLB value less than 10, for example 9, 8, 7, 6, 5, 4, 3, 2, 1 or 0.

It should be appreciated that the HLB value of a surfactant is merely a rough guide generally used to enable formulation of industrial, pharmaceutical and cosmetic emulsions. For many surfactants, including several polyethoxylated surfactants, it has been reported that HLB values can differ by as much as 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC® surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or lipophilicity for use in certain embodiments of the present invention.

Solubility enhancers can be any surfactant suitable for use in pharmaceutical compositions. Surfactants suitable for certain embodiments of the present invention can be anionic, cationic, zwitterionic or non-ionic. Examples of such surfactants can be grouped into the following general chemical classes detailed in Tables 1-18 herein. The HLB values given in Tables 1-18 below generally represent the HLB value as reported by the manufacturer of the corresponding commercial product. In cases where more than one commercial product is listed, the HLB value in the noted Tables is the value as reported for one of the commercial products, a rough average of the reported values, or a value that, in the judgment of the present inventors, is more reliable.

It should be emphasized that the surfactants of certain embodiments of the present invention are not limited to the surfactants listed in Tables 1-18, which show a list of non-exclusive examples of available surfactants. In addition, refined, distilled or fractionated surfactants, purified fractions thereof, or re-esterified fractions, are also within the scope of certain embodiments of the invention, although not specifically listed in the Tables.

Although polyethylene glycol (PEG) itself does not function as a surfactant, a variety of PEG-fatty acid esters have useful surfactant properties. Examples of polyethoxylated fatty acid monoester surfactants commercially available are shown in Table 1.

Polyethylene glycol (PEG) fatty acid diesters are also suitable for use as surfactants in the compositions of certain embodiments of the present invention. Representative PEG-fatty acid diesters are shown in Table 2.

In general, mixtures of surfactants are also useful in certain embodiments of the present invention, including mixtures of two or more commercial surfactant products. Several PEG-fatty acid esters are marketed commercially as mixtures or mono- and diesters. Representative surfactant mixtures are shown in Table 3.

Suitable PEG glycerol fatty acid esters are shown in Table 4.

A large number of surfactants of different degrees of lipophilicity or hydrophilicity can be prepared by reaction of alcohols or polyalcohols with a variety of natural and/or hydrogenated oils. In certain embodiments, the oils used are castor oil or hydrogenated castor oil or an edible vegetable oil such as corn oil, olive oil, peanut oil, palm kernel oil, apricot kernel oil, or almond oil. Non-limiting examples of alcohols include glycerol, propylene glycol, ethylene glycol, polyethylene glycol, sorbitol, and pentaerythritol. Representative surfactants of this class suitable for use in certain embodiments are shown in Table 5.

Polyglycerol esters of fatty acids are also suitable surfactants for certain embodiments of the present invention. Examples of suitable polyglyceryl esters are shown in Table 6.

Esters of propylene glycol and fatty acids are suitable surfactants for use in certain embodiments of the present invention. Examples of surfactants of this class are given in Table 7.

In general, mixtures of surfactants are also suitable for use in certain embodiments of the present invention. In particular, mixtures of propylene glycol fatty acid esters and glycerol fatty acid esters are suitable and are commercially available. Examples of these surfactants are shown in Table 8.

Another class of surfactants is the class of mono- and diglycerides. These surfactants are generally lipophilic. Examples of these surfactants are given in Table 9.

Sterols and derivatives of sterols are suitable surfactants for use in certain embodiments of the present invention. These surfactants can be hydrophilic or lipophilic. Examples of surfactants of this class are shown in Table 10.

A variety of PEG-sorbitan fatty acid esters are available and are suitable for use as surfactants in certain embodiments of the present invention. In general, these surfactants are hydrophilic, although several lipophilic surfactants of this class can be used. Examples of these surfactants are shown in Table 11.

Ethers of polyethylene glycol and alkyl alcohols are suitable surfactants for use in certain embodiments of the present invention. Examples of these surfactants are shown in Table 12.

Esters of sugars are suitable surfactants for use in certain embodiments of the present invention. Examples of such surfactants are shown in Table 13.

Several hydrophilic PEG-alkyl phenol surfactants are available, and are suitable for use in certain embodiments of the present invention. Examples of these surfactants are shown in Table 14.

The POE-POP block copolymers are a unique class of polymeric surfactants. The unique structure of the surfactants, with hydrophilic POE and lipophilic POP moieties in well-defined ratios and positions, provides a wide variety of surfactants suitable for use in certain embodiments of the present invention. These surfactants are available under various trade names, including SYNPERONIC™ PE series (ICI); PLURONIC® series (BASF), EMKALYX™, LUTROL™ (BASF), SUPRONIC™, MONOLAN™, PLURACARE™, and PLURODAC™. The generic term for these polymers is “poloxamer” (CAS 9003-11-6). These polymers have the formula:

HO(C₂H₄O)ₐ(C₃H₆O)_{b}(C₂H₄O)ₐH

where “a” and “b” denote the number of polyoxyethylene and polyoxypropylene units, respectively. In certain non-limiting examples, “a” can range from 0-130, 10-130, 20-130, 30-130, 40-130, 50-130, 60-130, 70-130, 80-130, 90-130, 100-130, 110-130, or 120-130, and “b” can range from 0-100, 10-100, 20-100, 30-100, 40-100, 50-100, 60-100, 70-100, 80-100, or 90-100. Examples of suitable surfactants of this class are shown in Table 15.

Sorbitan esters of fatty acids are suitable surfactants for use in certain embodiments of the present invention. Examples of these surfactants are shown in Table 16.

Esters of lower alcohols (C₂ to C₄, including C₃) and fatty acids (C₈ to C₁₈, including C₉, C₁₀, C₁₁, C₁₂, C₁₃, C₁₄, C₁₅, C₁₆, and C₁₇) are suitable surfactants for use in certain embodiments of the present invention. Examples of these surfactants are shown in Table 17.

Ionic surfactants, including cationic, anionic and zwitterionic surfactants, are suitable hydrophilic surfactants for use in certain embodiments of the present invention. In at least one embodiment the surfactant is an anionic surfactant such as a fatty acid salt, a bile salt, or a combination thereof. In other embodiments the surfactant is a cationic surfactant such as a carnitine. Non-limiting examples of ionic surfactants include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Examples of such surfactants are shown in Table 18.

Ionizable surfactants, when present in their unionized (neutral, non-salt) form, are lipophilic surfactants suitable for use in the compositions of certain embodiments of the present invention. Particular examples of such surfactants include free fatty acids, particularly C₆-C₂₂ fatty acids, including C₇, C₈, C₉, C₁₀, C₁₁, C₁₂, C₁₃, C₁₄, C₁₅, C₁₆, C₁₇, C₁₈, C₁₉, C₂₀, and C₂₁ fatty acids, and bile acids. More specifically, suitable unionized ionizable surfactants include the free fatty acid and bile acid forms of any of the fatty acid salts and bile salts shown in Table 18.

Derivatives, analogs, homologs, esters, amides, inorganic and organic salts, etc., of oil-soluble vitamins, such as vitamins A, D, E, K, etc., are also useful surfactants for the compositions of certain embodiments of the present invention. An example of such a derivative is tocopheryl PEG-1000 succinate (TPGS, available from Eastman).

In certain embodiments, surfactants or mixtures of surfactants that solidify at ambient room temperature are used. In other embodiments, surfactants or mixtures of surfactants that solidify at ambient room temperature in combination with particular lipophilic components, such as triglycerides, or with addition of appropriate additives, such as viscosity modifiers, binders, thickeners, and the like, are used.

Non-limiting examples of non-ionic hydrophilic surfactants include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated invention can be used in the microparticles, non-limiting examples of which include a lubricant, a binder, a pH modifier, a filler and/or a glidant.

In at least one embodiment the microparticle core comprises at least one of the following excipients: glyceryl monostearate, glyceryl behenate, glyceryl palmitostearate, carnauba wax, microcrystalline cellulose, lactose, and mixtures thereof.

The process for manufacturing the drug-containing microparticles of certain embodiments of the present invention is not limited to the CEFORM™ technology, and any other technology resulting in the formation of the drug-containing microparticles consistent with the present invention can also be used. For example, microparticles of other embodiments of the present invention can also be manufactured by extrusion/spheronization, granulation or pelletization.

Extrusion/spheronization is a multi-step process used to make uniformly sized spherical particles. The technique offers the ability to incorporate high levels of ingredients (e.g., drugs) without producing excessively large particles. The main steps in the process include: (i) Dry-mixing of ingredients to achieve a homogenous powder dispersion; (ii) Wet massing using for example a high-shear wet granulator to form rod shaped particles of uniform diameter; (iii) Extrusion to form rod-shaped particles of uniform diameter; (iv) Spheronization to round off the rods into spherical particles; and (v) Screening to achieve the desired narrow particle size distribution.

The mixing vessel used for dry-mixing can be of any size and shape compatible with the size of the formulation to be produced. For example, commercially available mixing devices such as planetary mixers, high shear mixers, or twin cone blenders can be used. If relatively small quantities of formulation are to be prepared, a simple mortar and pestle can be sufficient to mix the ingredients. The type of mixing vessel would be apparent to one skilled in the pharmaceutical art. The moistened mass formed by wet-massing in conventional granulation equipment is extruded through a perforated mesh in order to produce cylindrical filaments. The port of the meshes can determine the diameter of the filaments. A port ranging from about 0.2 mm to about 3 mm can be used in this process. In at least one embodiment lysophospholipids and derivatives thereof; salts of alkylsulfates; salts of fatty acids; sodium docusate; acyl lactylates; mono-,diacetylated tartaric acid esters of mono-,diglycerides; succinylated monoglycerides; citric acid esters of mono-diglycerides; carnitines; and mixtures thereof.

Non-limiting examples of ionic surfactants include lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

In certain embodiments, ionic surfactants used include lecithin, lysolecithin, phosphatidylcholine, phosphatidylethanolamine, phosphatidylglycerol, lysophosphatidylcholine, PEG-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, glycodeoxycholate, cholylsarcosine, caproate, caprylate, caprate, laurate, oleate, lauryl sulfate, docusate, and salts and mixtures thereof. In at least one embodiment, the ionic surfactant is selected from lecithin, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, taurocholate, caprylate, caprate, oleate, lauryl sulfate, docusate, and salts and mixtures thereof.

Non-limiting examples of lipophilic surfactants include alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; and mixtures thereof.

As with the hydrophilic surfactants, lipophilic surfactants can be reaction mixtures of polyols and fatty acids, glycerides, vegetable oils, hydrogenated vegetable oils, and sterols.

In certain embodiments, the lipophilic surfactants include one or more selected from the group consisting of fatty acids; lower alcohol fatty acid esters; polyethylene glycol glycerol fatty acid esters; polypropylene glycol fatty acid esters; polyoxyethylene glycerides; glycerol fatty acid esters; acetylated glycerol fatty acid esters; lactic acid derivatives of mono/diglycerides; sorbitan fatty acid esters; polyoxyethylene sorbitan fatty acid esters; polyoxyethylene-polyoxypropylene block copolymers; polyoxyethylene vegetable oils; polyoxyethylene hydrogenated vegetable oils; and reaction mixtures of polyols and fatty acids, glycerides, vegetable oils, hydrogenated vegetable oils, sterols, and mixtures thereof.

In certain other embodiments, the lipophilic surfactants include one or more selected from the group consisting of lower alcohol fatty acids esters; polypropylene glycol fatty acid esters; propylene glycol fatty acid esters; glycerol fatty acid esters; acetylated glycerol fatty acid esters; lactic acid derivatives of mono/diglycerides; sorbitan fatty acid esters; polyoxyethylene vegetable oils; and mixtures thereof. Among the glycerol fatty acid esters, the esters can be mono- or diglycerides, or mixtures of mono- and diglycerides, where the fatty acid is a C₆ to C₂₂ fatty acid.

Other embodiments include lipophilic surfactants which are the reaction mixture of polyols and fatty acids, glycerides, vegetable oils, hydrogenated vegetable oils, and sterols. Examples of polyols are polyethylene glycol, sorbitol, propylene glycol, pentaerythritol, and mixtures thereof. In at least one embodiment the surfactant used in the microparticle includes a polyethylene-polypropylene glycol that has an average molecular weight of from about 9,840 to about 14,600, from about 10,000 to about 14,600, from about 11,000 to about 14,600, from about 12,000 to about 14,600, from about 13,000 to about 14,600, or from about 14,000 to about 14,600. In at least one embodiment the surfactant used in the microparticle includes a polyethylene glycol that has an average molecular weight of from about 3,000 to about 4,800, from about 3,200 to about 4,800, from about 3,400 to about 4,800, from about 3,600 to about 4,800, from about 3,800 to about 4,800, from about 3,900 to about 4,800, from about 4,000 to about 4,800, from about 4,100 to about 4,800, from about 4,200 to about 4,800, from about 4,300 to about 4,800, from about 4,400 to about 4,800, from about 4,500 to about 4,800, from about 4,600 to about 4,800, or from about 4,700 to about 4,800.

Combinations of solubility enhancers (i.e. surfactants) can be used. Non-limiting examples of macrogol fatty acid esters useful as solubility enhancers in certain embodiments include GELUCIRE 50/13® and GELUCIRE 44/14®. In at least one embodiment the solubility enhancer is GELUCIRE 50/13®. In at least one embodiment the macrogol fatty acid esters used in the microparticle has a melting point of about 50° C., and a hydrophilic-lipophilic balance value of about 13.

The solubility enhancer can be present in an amount of from about 0% to about 90% by weight of the microparticle. For example, in certain embodiments, the solubility enhancer is present in an amount of from about 0.1% to about 50%, or in an amount of about 5%, about 10%, or about 20%; in other embodiments from about 30% to about 40%; and in still other embodiments about 35% by weight of the microparticle.

It is contemplated that in some embodiments, one or more other pharmaceutically acceptable excipients consistent with the objects of the present invention can be used in the microparticles, non-limiting examples of which include a lubricant, a binder, a pH modifier, a filler and/or a glidant.

In at least one embodiment the microparticle core comprises at least one of the following excipients: glyceryl monostearate, glyceryl behenate, glyceryl palmitostearate, carnauba wax, microcrystalline cellulose, lactose, and mixtures thereof.

The process for manufacturing the drug-containing microparticles of certain embodiments of the present invention is not limited to the CEFORM™ technology, and any other technology resulting in the formation of the drug-containing microparticles consistent with the present invention can also be used. For example, microparticles of other embodiments of the present invention can also be manufactured by extrusion/spheronization, granulation or pelletization.

Extrusion/spheronization is a multi-step process used to make uniformly sized spherical particles. The technique offers the ability to incorporate high levels of ingredients (e.g., drugs) without producing excessively large particles. The main steps in the process include: (i) Dry-mixing of ingredients to achieve a homogenous powder dispersion; (ii) Wet massing using for example a high-shear wet granulator to form rod shaped particles of uniform diameter; (iii) Extrusion to form rod-shaped particles of uniform diameter; (iv) Spheronization to round off the rods into spherical particles; and (v) Screening to achieve the desired narrow particle size distribution.

The mixing vessel used for dry-mixing can be of any size and shape compatible with the size of the formulation to be produced. For example, commercially available mixing devices such as planetary mixers, high shear mixers, or twin cone blenders can be used. If relatively small quantities of formulation are to be prepared, a simple mortar and pestle can be sufficient to mix the ingredients. The type of mixing vessel would be apparent to one skilled in the pharmaceutical art. The moistened mass formed by wet-massing in conventional granulation equipment is extruded through a perforated mesh in order to produce cylindrical filaments. The port of the meshes can determine the diameter of the filaments. A port ranging from about 0.2 mm to about 3 mm can be used in this process. In at least one embodiment utilizing this process, the port ranges from about 0.4 mm to about 2 mm. The extrusion can be carried out using screw, double screw, “sieve and basket” kind, “roll extruder”, “ram extruder” extruders or any other pharmaceutically acceptable means to produce cylindrical filaments. In certain embodiments utilizing this extrusion/spheronization process, a double screw coaxial extruder is used. The spheronization device comprises a hollow cylinder with a horizontal rotating plate. The filaments are broken in short segments which are transformed in spherical or quasi-spherical particles on the upper surface of the rotating plate at a velocity ranging from about 200 rpm to about 2,000 rpm, for example about 500 rpm, about 750 rpm, about 1,000 rpm, about 1,250 rpm, about 1,500 rpm, or about 1,750 rpm. The particles can be dried in any pharmaceutically acceptable way, such as for example by air drying in a static condition. The particles are used as they are or they are coated to obtain granules to use in tablets, capsules, packets or other pharmaceutical formulations.

An example of an extrusion/spheronization formulation of the present invention can be as follows: In this prophetic example, the drug can be present in an amount of from about 1% to about 80% w/w. In certain embodiments the drug is present in an amount of from about 1% to about 50% w/w; in other embodiments from about 10% to about 30%; and in still other embodiments at about 10% w/w. In this example the filler can be present in an amount of from about 0% to about 80% w/w. In certain embodiments the filler is present in an amount of from about 10% to about 60%; and in other embodiments at about 40% w/w. In this example microcrystalline cellulose can be present in an amount of from about 10% to about 90% w/w. In certain embodiments the microcrystalline cellulose is present in an amount of from about 10% to about 70%; and in other embodiments from about 20% to about 50% w/w. In this example the binder can be present in an amount of from about 0% to about 10% w/w. In certain embodiments the binder is present in an amount of from about 1% to about 8%; and in other embodiments from about 2% to about 4% w/w. In this example water can be present in an amount of from about 10% to about 80% w/w. In certain embodiments water is present in an amount of from about 15% to about 70%; and in other embodiments from about 20% to about 50% w/w. Suitable fillers in this example include but are not limited to calcium phosphate dibasic, tricalcium phosphate, calcium carbonate, starch (such as corn, maize, potato and rice starches), modified starches (such as carboxymethyl starch, etc.), microcrystalline cellulose, sucrose, dextrose, maltodextrins, lactose, and fructose. Suitable lubricants in this example include but are not limited to metal stearates (such as calcium, magnesium on zinc stearates), stearic acid, hydrogenated vegetable oils, talc, starch, light mineral oil, sodium benzoate, sodium chloride, sodium lauryl sulfate, magnesium lauryl sulfate, sodium stearyl fumarate, glyceryl behenate and polyethylene glycol (such as CARBOWAX™ 4000 and 6000). Suitable antiadherents in this example include but are not limited to colloidal silicon dioxide. Suitable binders in this example include but are not limited to ethyl cellulose, a polymethacrylate polymer, polyvinylalcohol, polyvinyl pyrrolidone, polyvinylpyrrolidone-vinylacetate copolymer (e.g. KOLLIDON™ VA64) hydroxyethylcellulose, low molecular weight hydroxypropylmethylcellulose (e.g. viscosity of from about 1 to about 50 cps at about 20° C., for example about 10 cps, about 20 cps, about 30 cps, or about 40 cps; from about 2 to about 12 cps at about 20° C., for example about 5 cps, about 7 cps, about 8 cps, or about 10 cps; or from about 4 to about 6 cps at about 20° C.), hydroxypropylcellulose polymethacrylates, and mixtures thereof.

The drug-containing microparticles formed by extrusion/spheronization in this prophetic example can be produced using cross-linked amphiphilic polymers by the following steps: (a) the mixing of one or more cross-linked amphiphilic polymers with the drug and optionally other pharmaceutical excipients in order to obtain a uniform mixture in the form of dry powder to which a suitable amount of liquid is added to obtain a pasty consistency; (b) the extrusion of the mixture obtained from step (a) through a perforated mesh in order to obtain cylindrical filaments having desired length and diameter; (c) the spheronization of the filaments in order to obtain a product in the form of spherical multiparticulates; (d) the drying of the product; and (e) the optional depositing of a drug on the surface of the microparticles. “Cross-linked amphiphilic polymer” in this example refers to polymers showing characteristics of swellability in the whole pH range of aqueous solutions and also in solvents or solvent mixtures having different polarity characteristics. The polymers can be cross-linked either physically through the interpenetration of the macromolecular meshes, or chemically, thus showing points of link among the macromolecular chains. Non-limiting examples of such polymers include cross-linked polyvinyl pyrrolidone, sodium carboxymethylcellulose, sodium glycolate starch and dextrans. Optional excipients include dispersing, emulsifying, wetting agents and colouring agents. The expression “uniform mixture” in this example means that the components of the mixture are uniformly dispersed in the formulation by a mixing process which provides the uniform distribution of each component. A reasonable mixing time in this example can range from about 1 to about 60 minutes, for example about 5 minutes, about 10 minutes, about 20 minutes, about 30 minutes, about 40 minutes, or about 50 minutes using one of the mixing equipments conventionally used for the dry mixing of the powders (e.g. “V”, fixed body, rotating body, sigma mixers). The term “liquid” in this example means any liquid substance or mix (solution or emulsion) of liquids of normal pharmaceutical use able to moisten the powder mix, as for example water, aqueous solutions having different pH, organic solvents of normal pharmaceutical use (e.g. alcohols, chlorinated solvents), and oils. Among the oils and surfactants which can be used in this example are: natural oils, either saturated or unsaturated (olive, peanut, soybean, corn, coconut, palm, sesame and similar oils); semisynthetic and synthetic mono-, di- and triglycerides containing saturated and/or unsaturated fatty acids and their polyhydroxyethylated derivatives (caprico-caprilic triglycerides [MYGLIOL™, CAPTEX™, LABRAFAC™, Lipo], saturated or unsaturated polyhydroxylated triglycerides of various kind [LABRAFIL™, LABRAFAC™ Hydro, GELUCIRE™]); liquid waxes (isopropyl myristate, isopropyl-caprinate, -caprylate, -laurate, -palmitate, -stearate); fatty acids esters (ethyl oleate, oleyl oleate); silicone oils; polyethylene glycols (PEG 200, PEG 400, PEG 600, PEG 1000, and so on); polyglycolic glycerides (for example LABRASOL™); polyglycols propylene glycol, tetraglycol, and ethoxydiglycol (TRANSCUTOL™), sorbitan-esters of fatty acids (for example SPAN®, ARLACEL®, BRIJ®), polyoxyethylenesorbitan esters of fatty acids (for example TWEEN®, CAPMUL®, LIPOSORB®), polypropylene oxide-polyethylene oxide (Poloxamer) copolymers, polyethylene glycol esters (PEG)-glycerol (LABRASOL®, LABRAFIL®), PEG esters and long chain aliphatic acids or alcohols (for example CREMOPHOR®), polyglycerid esters (PLUROL®), saccharide and fatty acid esters (sucro-esters). Moreover, anionic surfactants (for example sodium lauryl sulfate, sodium stearate, sodium oleate) or cationic surfactants (for example tricetol), can be used as well as lecithins, phospholipids and their semi-synthetic or synthetic derivatives. Moreover certain drugs and/or excipients can be dissolved, dispersed and/or emulsified in such liquids.

In certain embodiments formed by an extrusion/spheronization process from the example described above, the moistening liquid comprises an oil/surfactant system wherein the drug optionally emulsified with an aqueous phase is dissolved or dispersed. The amount of liquid with respect to the solid used in the preparation of the mixture can range from about 1% to about 80% by weight. In this example, a mixture of drug and KOLLIDON™ CL in a ratio equal to about ⅓ by weight can be comilled to obtain the mixture in the form of powder having about 100% of granulometry lower than about 50 micron. The mixture can be moistened using a liquid demineralized water containing KOLLIDON™ 25 (polyvinyl pyrrolidone, Basf) in a solution 3% w/w. The extrusion can be carried out forcing the moistened mass through a threader having diameter of the holes equal to about 1 mm. The operative parameters in this example can be as follows: powder flow rate: about 4.5 kg/h; liquid flow rate: about 4.1 kg/h; torsional stress: about 27%; head temperature: about 46° C.; and screw rotation velocity: about 140 rpm. The extrusion filaments are then processed in a spheronizator adjusted at a velocity equal to about 1,000 rpm for about 2 minutes. The obtained microparticles can then be dried in a fluid bed for about 2 hours to a maximum temperature equal to about 59° C. At the end of the drying the product can be discharged and mechanically screened to separate the fraction ranging from about 0.7 mm to about 1.2 mm

Another prophetic example of a drug-containing microparticle of the present invention formed by an extrusion/spheronization process, uses a charged resin, the steps of which can comprise: (a) Adding the charged resin, drug and other excipients, to a mixing vessel; (b) Mixing the ingredients to obtain a uniform mixture; (c) Adding a granulating solution—a liquid capable of wetting the dry mixture. Liquids resulting in conversion of the dry powder mixture into a wet granulation that supports subsequent extrusion and marumerization can be included. Water or aqueous solutions can be employed. Alcohols, (e.g. ethanol or isopropanol), can be included with the granulating water to enhance the workability of the granulation. In certain embodiments one or more of the components of the formulation is first dissolved in water and this solution can then be used to produce the wet granulation. A drug or an excipient which is present at very low concentration can initially be dissolved or suspended in the granulating solvent to provide more uniform distribution throughout the formulation. (d) Granulating the mixture until a uniform granulation results; (e) Extruding the wet granulation through a screen to produce strands of granulation; (f) Spheronizing the strands of granulation to produce spherical multiparticulates; and (g) Collecting and drying the spherical multiparticulates. “Charged resin” is meant to mean a polymer with ionizable functional groups that can become useful in this example of a drug-containing microparticle. This broadly encompasses any polymer that upon ionization, is capable of producing cationic or anionic polymeric chains and which support spheronization. For example, from about 10% to about 70% by weight of the spherical multiparticulate can be charged resin. Non limiting examples of these charged resins include sodium polystyrene sulfonate which is sold under the trade name AMBERLITE IRP-69™ by Rohm and Haas, Co., Philadelphia, Pa.; the chloride salt of cholestyramine resin USP, sold as AMBERLITE IRP-276™ by Rohm and Haas, Co., Philadelphia, Pa.; the acid form of methacrylic acid-divinyl benzene, sold as AMBERLITE IRP-64™ by Rohm and Haas Co., Philadelphia, Pa.; carboxypolymethylenes sold under the trade names CARBOPOL™ 974P and CARBOPOL™ 934P by B. F. Goodrich, Inc., Brecksville, Ohio, and sodium polyacrylate, sold under the trade name AQUAKEEP™ J-550 by Seitetsu Kagaku, Japan. In order for the resin to maintain the desired degree of ionization, agents which produce an acidic or basic environment during granulation and spheronization can be included within the formulation. Among the groups of compounds that can exert this effect are acids, bases, and the salts of acids and bases such as adipic acid, citric acid, fumaric acid, tartaric acid, succinic acid, sodium carbonate, sodium bicarbonate, sodium citrate, sodium acetate, sodium phosphates, potassium phosphates, ammonium phosphate, magnesium oxide, magnesium hydroxide, sodium tartrate, and tromethamine. Certain compounds can be added to the granulation to provide the proper degree of hydration of the charged resin, medicament and excipients. These hydrating agents include sugars such as lactose, sucrose, mannitol, sorbitol, pentaerythritol, glucose and dextrose. Polymers such as polyethylene glycol as well as surfactants and other organic and inorganic salts can also be used to modulate polymer hydration.

In another example, multiparticulates of certain embodiments of the present invention can be obtained using the following components: drug; disodium phosphate; monosodium phosphate; sodium dodecyl sulfate; sodium chloride; POVIDONE 29-32K™; AMBERLITE IRP-69™; and butylated hydroxyanisol. In this prophetic example, approximately 5.75 kg of the above formulation is mixed in a planetary mixer for about 15 minutes. The butylated hydroxyanisol is dissolved in about 60 cc of ethanol and water is added to bring the final solution to a volume of about 133 cc. This solution is added to the planetary mixer over about a two minute period. The mixer is then granulated with seven aliquots of about 250 cc of water added over about a fifteen minute period. The granulation thus formed is extruded through about a 1.0 mm screen and aliquots spheronized by marumerization at about 1200 rpm for about 10 minutes each. The spherical multiparticulates formed are then dried at about 50° C. for about 24 hours.

Other embodiments of this invention involve the production of drug containing microparticles in the form of ‘pearls’. Pearls can be manufactured by mixing drug with one or more pharmaceutical excipients in molten form. In a prophetic example the melt is forced to pass through a nozzle which is subjected to a vibration. The pearls formed are allowed to fall in a tower countercurrentwise to a gas, and the solid pearls are then collected in the bottom of the tower. In this example, the quantity of drug can vary from about 5% to about 95% by weight; and in certain embodiments from about 40% to about 60% by weight. The additives which enable the crystallization of the supercooled product to be induced in this example can be chosen from the following: fatty alcohols such as: cetyl alcohol, stearyl alcohol, fatty acids such as: stearic acid, palmitic acid, glycerol esters such as: glycerol palmitostearate, the glycerol stearate marketed under the mark PRECIROL™, the glycerol behenate marketed under the mark COMPRITOL™, hydrogenated oils such as: hydrogenated castor oil marketed under the mark CUTINA™ HR, fatty acid salts such as: magnesium or calcium stearate, polyols such as: mannitol, sorbitol, xylitol, waxes such as: white wax, carnauba wax, paraffin wax, polyoxyethylene glycols of high molecular weight, and esterified polyoxyethylenes such as: PEG-32 distearate, and PEG-150 distearate. To these crystallization additives it can be desirable in this example to add polymers which are soluble or dispersible in the melt, and which provide a controlled and adjustable dissolution of the pearls when they are used, examples of which include: cellulose derivatives (hydroxypropyl cellulose, hydroxypropyl methyl cellulose, hydroxyethyl cellulose, ethyl cellulose, carboxymethyl cellulose), acrylic resins (marketed under the mark EUDRAGIT®), polyvinyl acetates (marketed under the mark RHODOPAS®), polyalkylene (ethylene propylene), polylactic, maleic anhydride and silicone resins. In addition, inorganic additives can be added to accelerate the solidification of the drug, examples of which include: silicas, inorganic oxides such as titanium or iron oxide, phosphates, carbonates, clays, and talc. In addition, a surface-active agent can be added to improve the dispersion of the drug in the crystallization additive, examples of which include: sorbitol esters, the polyoxyethylene polysorbates marketed under the mark TWEEN®, and glycols such as glycerine or propylene glycol. The process for the preparation of pearls comprise preparing a melt of the drug with one or more excipients. This melt can be prepared by separately melting the various constituents and then mixing them or by melting the mixture of the constituents, possible insoluble compounds being added at the end of the melting so as to obtain a homogeneous mass. The nature of the constituents of the melt is considered as a function of the compatibility of the constituents, the viscosity of the mixture of constituents, the nozzle diameter, the hydrophilicity of the drug, the surface tension of the drug, the particle size of the insoluble additives, the flow rate of the nozzle, the temperature of the tower, its height, the size of the desired pearls, the proportion of drug to be included therein and the desired release time of the drug.

Alternative procedures (other than extrusion or spheronization) for manufacturing drug-containing microparticles of the present invention can include wet granulation, solvent granulation and melt granulation. All of these techniques involve the addition of an inactive binder to aggregate smaller particles into larger granules. For example, wet granulation and solvent granulation involve the addition of a liquid binder which aggregates the drug materials and excipients into granules. After granulation, the liquid can be removed by a separate drying step. Melt granulation is similar to wet granulation, but uses a low melting point solid material as a binder. The solid binder in melt granulation is melted and acts as a liquid binder thereby aggregating the powdered drug material and excipients into granules. The binder thereby, can be incorporated into the granules when the granules cool.

Certain embodiments of the present invention include microparticles manufactured by a process for producing granules by rotomelt granulation that comprises mixing the drug and a powdered excipient material that has a higher melting point than the drug in a zone wherein both powdered materials are maintained in a fluidized state by a rising stream of gas in an apparatus having a rapidly rotating horizontal-disk located within a vertical vessel having a bottom surface. The rapidly rotating disk can be located on the bottom surface of the vertical vessel wherein the gas is at a temperature sufficient to cause the drug to at least partially melt thereby causing the powdered materials to aggregate and form granules. Other embodiments of the present invention include microparticles manufactured by a process for producing granules by rotomelt granulation comprising mixing powdered binder material and drug, wherein the drug has a higher melting point than the powdered binder material in a zone wherein both powdered materials are maintained in a fluidized state by a rising stream of gas in an apparatus having a rapidly rotating horizontal-disk located within a vertical vessel having a bottom surface. The rapidly rotating disk can be located on the bottom surface of the vertical vessel wherein the gas is at a temperature sufficient to cause the powdered binder material to at least partially melt thereby causing the powdered materials to aggregate and form granules.

In rotomelt granulation, one of the feed powders, in order to serve as a binder, has a lower melting point than the other powder. The feed powders are introduced into a vertical vessel with rotatable horizontal-disk located in the bottom of the vessel. The powder is maintained in fluidized state by at least one stream of filtered air being circulated from the bottom of the vertical vessel through one or more inlets. The rotatable horizontal disk is then rotated while the air supplied to fluidize the powder is maintained at a temperature sufficient to soften or melt the lower melting point powder. The temperature to which the binder must be heated to soften can be empirically determined by observing the formation of granules at various temperatures for various binders. Temperatures from about 3° C. to about 5° C. below the melting point or melting range can provide sufficient softening to result in granule formation. The lower melting point powder then acts as a binding agent to promote the aggregation of powder particles into granules. Suitable powders for use in rotomelt granulation have a diameter size in the range of from about 5 micron to about 150 micron, for example about 10 micron, about 20 micron, about 30 micron, about 40 micron, about 50 micron, about 60 micron, about 70 micron, about 80 micron, about 90 micron, about 100 micron, about 110 micron, about 120 micron, about 130 micron, or about 140 micron; and in certain embodiments have a diameter size in the range of from about 35 micron to about 80 micron. The temperature which the components will be exposed to depends at least in part on the binder employed to aggregate the powders. Generally, the melting point of the binder is above about 30° C.; and in certain embodiments is below about 100° C.

The powders used in these microparticles manufactured by rotomelt granulation can be formed into granules by at least two alternative granulation mechanisms. The first mechanism for granule formation utilizes a larger particulate binder and a smaller particulate powder. The temperature during the rotomelt granulation is then elevated only to the point where the external surface of the binder particles become tacky. As the second powdered material of a smaller size is contacted with the tacky surface it forms a microlayer on the surface of the binder particle. This granulation mechanism results in granules which have size distribution similar to the original binder particles employed. Alternatively, the rotomelt granulation can be conducted at a temperature at which the binder acts as a cement bridging the gaps between the unmelted particles (this is referred to as agglomeration). This mechanism results in the formation of granules where the components are intermingled. For each binder used the mechanism can be controlled primarily by the temperature at which the rotomelt granulation is performed. The granules formed can be observed by electron microscopy to determine the type of granulation process occurring. If one particular type of granule is desired, the process conditions or starting materials can be varied to produce the desired granules.

In at least one embodiment of the present invention, the drug is melted to act as a binding agent in the rotomelt granulation process. Non-limiting examples of suitable excipients include those selected from the following: fillers, lubricants and antiadherents. Suitable fillers include but are not limited to calcium phosphate dibasic, tricalcium phosphate, calcium carbonate, starch (such as corn, maize, potato and rice starches), modified starches (such as carboxymethyl starch, etc.), microcrystalline cellulose, sucrose, dextrose, maltodextrins, lactose, and fructose. The amount of binder added to aggregate the particles into granules can be in the range of from about 10% w/w to about 80% w/w; and in certain embodiments is in the range of from about 30% w/w to about 70% w/w of the powdered materials in the rotomelt granulation. The remaining weight percentage to provide a total of 100% w/w can be one or more suitable powdered pharmaceutical drugs. Optionally the rotomelt granulation can also contain from about 0% to about 60% w/w, for example about 10% w/w, about 20% w/w, about 30% w/w, about 40% w/w/, or about 50% w/w of one or more powdered excipients wherein the total weight of all the powdered materials equals 100% w/w. The binder used in these embodiment of the invention can be a pharmaceutically acceptable dry powder having a particle size in the range of from about 5 μm to about 150 μm, for example about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, or about 140 μm; and in certain embodiments in the range of from about 35 μm to about 80 μm. Suitable binders for rotomelt granulation are low melting point powdered binders, non-limiting examples of which include: polyethylene glycol 4000, polyethylene glycol 6000, stearic acid, and low melting point waxes. Suitable low melting point waxes include but are not limited to glyceryl monostearate, hydrogenated tallow, myristyl alcohol, myristic acid, stearyl alcohol, substituted monoglycerides, substituted diglycerides, substituted triglycerides, white beeswax, carnauba wax, castor wax, japan wax, acetylate monoglycerides and combinations thereof. The binders can have a melting point of from about 30° C. to about 100° C.; and in certain embodiments from about 40° C. to about 85° C.

As a prophetic example of these embodiments that are manufactured by a rotomelt granulation process, about 320 g of drug and about 80 g PEG 8000 is dry blended and poured into a Glatt 1.1 chamber set-up as a rotary granulator with a longitudinal plate. Inlet air temperature is set to about 60° C. and the product chamber heated to about 50° C. The blend is fluidized at about 120 m3/hr and the frictional plate set to about 900 rpm. The product chamber temperature is raised to about 60° C. and then gradually reduced to about 20° C. over a period of about 20 minutes, during which spheronization is achieved.

Other embodiments of the invention involve the formation of a microparticle that has a core which includes the drug and a compound which is sweet in taste and which has a negative heat of solution. Non-limiting examples of compounds falling into this category include mannitol and sorbitol. Sugars or artificial sweeteners to which, for example, menthol have been added can also work as well. A binder and/or other excipient can also be disposed within the core. The amount of sweetening compound used can depend on a number of factors including the size of the resulting microparticles, the size or volume of the resulting tablet, the sturdiness of the microparticle-coated microparticulant, the speed at which the tablet will disintegrate in the mouth, the degree of sweetness imparted by the particular sweetener used, either in the microparticle or in the tablet, or both, the amount of drug used, and the like. For example, particularly rugged microparticles can be less likely to break during chewing and/or compression. Therefore, the amount of material provided to protect against the release of objectionably flavored material can be lessened. In other cases a greater relative amount of sweetening compound can be used. In certain embodiments the amount of sweetening material used can range from greater than zero to about 80%, for example about 10%, about 20%, about 30%, about 40%, about 50%, about 60%, or about 70% of the weight of the resulting microparticle. The sweetener and drug can be combined in any number of known ways, such as for example by wet granulation, dry granulation, agglomeration, or spray coating. For example, the sweetener can be used as an adsorbent for the drug. Alternatively, particles of each can also be simply mixed together. One or more binders, or other adjuvants can also be used in the formulation of a tablet as well. Binders in these embodiments include, for example: starch (for example, in an amount of from about 5% to about 10% as an aqueous paste); pregelatinized starch (for example, in an amount of from about 5% to about 10% added dry to powder); gelatin (for example, in an amount of from about 2% to about 10% as an aqueous solution, or about 2% in starch paste); polyvinylpyrrolidone (for example, in an amount of from about 2% to about 20% in an aqueous or alcoholic solution); methylcellulose (for example, in an amount of from about 2% to about 10% as an aqueous solution); sodium carboxy methylcellulose (for example, in an amount of from about 2% to about 10% as an aqueous solution); ethylcellulose (for example, in an amount of from about 5% to about 10% as an alcohol or hydroalcoholic solution); polyacrylamides (Polymer JR) (for example, in an amount of from about 2% to about 8% as an aqueous solution); polyvinyloxoazolidone (Devlex) (for example, in an amount of from about 5% to about 10% as an aqueous or hydroalcoholic solution); polyvinyl alcohols (for example, in an amount of from about 5% to about 20% in aqueous solutions); and mixtures thereof. Other adjuvants can also be used in forming the core of the microparticles of the present embodiments of the invention, non-limiting examples of which include: calcium sulfate NF, Dibasic Calcium phosphate NF, Tribasic calcium sulfate NF, starch, calcium carbonate, microcrystalline cellulose, modified starches, lactose, sucrose and the like, STA-RX™, AVICEL™, SOLKA-FLOC™ BW40, Alginic acid, EXPLOTAB™, AUTOTAB™, guar gum, kaolin, VECGUM™, bentonite, and mixtures thereof. These adjuvants can be used in up to about 20% w/w; and in certain embodiments are present in an amount of from about 3% to about 5% w/w.

As a prophetic example of these embodiments that have a core comprising drug and a compound which is sweet in taste, the drug can be granulated using the following procedure: Polyvinylpyrrolidone K-30 USP (e.g. about 240.0 gm) is dissolved into distilled water (e.g. about 1,890.0 gm) with agitation. Mannitol powder USP (e.g. about 11,160 gm) and drug (e.g. about 600.0 gm) are placed in a Zanchetta 50-liter granulator/processor. After an initial two-minute dry mix of the powders with the chopper on and the propeller adjusted to about 200 rpm, the polyvinylpyrrolidone K-30 solution is slowly sprayed into the mixing powder bed using an air-driven spray system. The total time of granulation including the time of solution addition is about eight minutes. The granulation end-point is determined visually and by the consistency of the resulting material. The material is then discharged onto trays and dried at about 80° C. utilizing supplied dry air for a period of about six hours to a moisture content of not more than about 0.08 percent. The dried material is then passed through a hammermill (knives forward) equipped with a U.S. #40 (e.g. about 420 micron) screen.

Other embodiments of this invention involve the combined granulation and coating of drug into microparticles in which the drug is located at least within the microparticle core but capable of immediate release. In these embodiments, the drug and a granular disintegrant are first dry-mixed; the powder obtained is then granulated, in the presence of a mixture of excipients comprising at least one binder capable of binding the particles together to give grains; the grains thus formed are then coated by spraying with a suspension comprising at least one coating agent and a membrane disintegrant; and then the coated granules obtained are dried. In these embodiments, the distinction between the actual granulation and coating steps can be relatively theoretical, insofar as, even though the primary function of the binder used in the granulation step is to bind together the particles, it nevertheless already partially coats the grains formed. Similarly, even though the primary function of the coating agent used in the actual coating step is to complete the final coating of each of the grains, it may, however, arbitrarily bind other coated grains by a mechanism of granular agglomeration. The binder and the coating agent can be chosen from the group comprising cellulose polymers and acrylic polymers. However, even though the binder and the coating agent can be chosen from the same group of compounds, they nevertheless differ from each other in their function as previously mentioned. Non-limiting examples of the cellulose polymers that can be advantageously chosen include ethylcellulose, hydroxypropylcellulose (HPC), carboxymethylcellulose (CMC) and hydroxypropylmethylcellulose (HPMC), and mixtures thereof. Non-limiting examples of the acrylic polymers that can be advantageously chosen include the ammonio-methacrylate copolymer (EUDRAGIT® RL or RS), the polyacrylate (EUDRAGIT® NE), the methacrylic acid copolymer (EUDRAGIT® L or S), and mixtures thereof. In at least one embodiment, the binder is of the same nature as the coating agent. To further accelerate the release of the drug, the coating suspension of certain embodiments can also comprise a permeabilizer which, on account of its intrinsic solubility properties, can cause perforation of the membrane coating, thus allowing the drug to be released. Non-limiting examples of permeabilizers include povidone and its derivatives, polyethylene glycol, silica, polyols, low-viscosity cellulose polymers, and mixtures thereof. Polymers such as hypromellose, whose viscosity is equal to about 6 centipoises, can be used, for example, as a low-viscosity cellulose polymer. In at least one embodiment, the dry-mixing of initial powder and the granulation, coating and drying steps are performed in a fluidized bed. In these embodiments the initial powder mixture is first fluidized before being granulated by spraying said powder with the excipient mixture comprising at least the binder; the grains obtained then being coated by spraying with the coating suspension; the coated granules formed finally being dried in the fluidized bed. In at least one embodiment, the mixture of excipients used during the granulation step and the coating suspension used during the coating step form a single mixture. In these embodiments the granulation step can be distinguished from the spraying step by varying different parameters, such as the rate of spraying of the mixture and the atomization pressure of said mixture. Thus in certain embodiments, only some of the mixture of excipients is used during the granulation step, while the other portion can be used during the coating step. Thus, the rate of spraying of the coating suspension is higher during the granulation step than during the coating step, whereas the atomization pressure of the coating suspension is lower during the granulation step than during the coating step. At the laboratory scale in a fluidized-bed device, for example of the type such as Glatt GPCG1, during the granulation step, the rate of spraying of the coating suspension is between about 10 grams/minute and about 25 grams/minute, for example about 15 grams/minute or about 20 grams/minute, and the atomization pressure is between about 1 bar and about 1.8 bar, for example about 1.2 bar, about 1.4 bar, or about 1.6 bar. During the coating step, the rate of spraying of the coating suspension is between about 5 grams/minute and about 15 grams/minute, for example about 7 grams/minute, about 9 grams/minute, about 11 grams/minute, or about 13 grams/minute while the atomization pressure is between about 1.5 bar and about 2.5 bar, for example about 1.7 bar, about 1.9 bar, about 2.1 bar, or about 2.3 bar. In at least one embodiment, between about 10% and about 20%, for example about 12%, about 14%, about 16%, or about 18% of the mixture of excipients is sprayed during the granulation step, the remainder being sprayed during the coating step.

As a prophetic example of these embodiments that involve the combined granulation and coating of drug into microparticles in which the drug is located at least within the microparticle core but capable of immediate release, the microparticles can be manufactured according to the following process: A granulation solution is first prepared by dissolving about 48 g of ethylcellulose in about 273 g of ethyl alcohol. A coating suspension is then prepared by mixing about 97 g of ethylcellulose, about 28.5 g of polyethylene glycol 6000, about 26 g of sodium croscarmellose, about 10 g of precipitated silica and about 27.5 g of aspartam in about 1900 g of ethyl alcohol, until a homogeneous suspension is obtained. The powder mixture comprising about 700 grams of drug and about 35 grams of Acdisol is then fluidized. The granulation is then started by spraying the granulation solution for about 15 to about 20 minutes at a spraying rate of about 25 grams/minute and a suspension atomization pressure of about 0.8 bar. The actual coating is then performed by spraying the coating suspension for about 1 hour 30 minutes at a spraying rate of from about 15 to about 20 grams/minute and a suspension spraying pressure of about 1.5 bar.

Certain other embodiments of the invention involve coating the drug material, thereby forming a drug-containing microparticle. One such process for achieving this involves: (i) Blending and fluidizing a powder mix of drug and an adjuvant in order to obtain individual grains; (ii) Separately liquifying under warm conditions a lipid matrix agent comprising either an ester of behenic acid and alcohol or an ester of palmitic/stearic acid and alcohol; (iii) Coating the fluidized powder mix under warm conditions by spraying the lipid matrix agent over the individual grains; and (iv) Lowering the temperature of the combined product in order to allow the lipid matrix agent to solidify. This process does not require an evaporation phase or a drying phase, since it does not require a wet-route or solvent-route granulation step, thus making it possible to be freed from any risk due to the presence of toxic residues in the final product. Furthermore, it is not necessary to carry out the quantitative determination of the traces of solvents, an analysis that can be very expensive. According to the process of these embodiments, the spraying conditions and thus the coating characteristics can be modified, in order to vary the release profile of the drug, by varying several parameters, the adjustment characteristics of which can remain simple. Thus, the spraying air pressure can be increased in order to promote the formation of a homogeneous film of lipid matrix agent around the grains. The rate of spraying of the lipid matrix agent can simultaneously be decreased. In this case, the drug release profile, that is to say a percentage of dissolution as a function of the time, is obtained which can be low, corresponding to a slow release of the drug. Conversely, the spraying air pressure can be decreased in order to promote the agglomeration of the grains with one another. The rate of spraying of the lipid matrix agent can simultaneously be increased. In this case, a release profile of the grains obtained can be obtained which is high, corresponding to a rapid release of drug. In these embodiments and according to the mass of powder employed, the value of the rate of spraying of the lipid matrix agent can be from two to four times higher when it is desired to promote the agglomeration of the grains with one another than when it is desired to promote the formation of a homogeneous film around the grains. On the other hand, the value of the spraying air pressure can be from one to two times lower when it is desired to promote the agglomeration of the grains with one another than when it is desired to promote the formation of a homogeneous film around the grains. It is possible, after having determined a given drug release profile, to vary the values of spraying air pressure and of spraying rate throughout the coating stage, making it possible to promote the formation of a homogeneous film around the grains or to promote the agglomeration of the grains. Once the sequence of the duration of the spraying air pressure and of the spraying rate has been determined, the coating operation can be carried out continuously and automatically. In certain embodiments the temperature of the mixture of liquefied matrix agent and of spraying air is greater by about 35° C. to about 60° C. than the melting temperature of the lipid matrix agent; and likewise, the temperature of the fluidization air and that of the powder is approximately equal to the melting temperature of the lipid matrix agent, plus or minus about 10° C. Furthermore, in order to obtain a mixture of individual grains, an air-operated fluidized bed device or a turbine device can be used. Furthermore, the lipid matrix agent can be sprayed by the air spray technique, that is to say liquid spraying under pressure in the presence of compressed air. According to at least one embodiment, use is made of a powder comprising the drug and the adjuvant. In other words, after mixing and fluidizing the combined constituents of the powder, the lipid matrix agent is sprayed over the individual grains obtained. In order to avoid adhesion of the coated grains obtained, whether in the case where all the grains are treated or whether in the case where only a portion of the grains is treated, a stage of lubrication of the grains is inserted between the coating stage and the stage of putting into a pharmaceutical form. Furthermore, in order to obtain greater stability (e.g. minimize modifications relating to the release of the drug over time) of the composition, the granules or tablets obtained in certain embodiments of this example can be subjected to a maturing stage in an oven, for at least about 8 hours, at a temperature of between about 45° C. and about 60° C.; and in certain embodiments at about 55° C.

As a prophetic example of these drug-containing microparticle embodiments that are formed by coating the drug material, the drug-containing microparticles can be manufactured according to the following process: A mixture of about 3 kg of powder is prepared comprising: about 1920 g of drug; about 90 g of dicalcium phosphate dehydrate; and about 90 g of polyvinylpyrrolidone. Batches of granules are prepared by a process comprising the following stages: the mixture of powder obtained is sieved; the said powder is mixed, heating while by means of an air-operated fluidized bed, in order to obtain individual grains; the lipid matrix agent (glyceryl behenate, sold under the trade name COMPRITOL® 880 ATO) is liquefied separately at about 120° C.; the lipid matrix agent is sprayed over the heated powder mixture, and, finally, the temperature is lowered in order to allow the lipid matrix agent to solidify. These stages are carried out while varying various parameters, either in order to promote the formation of a homogeneous film around the grains or in order to promote the agglomeration of the grains, in accordance with the following table:

| PARAMETERS | Batch 1 | Batch 2 | Batch 3 | Batch 4 |
|---|---|---|---|---|
| % by weight of lipid matrix | | | | |
| agent (COMPRITOL ® 888 ATO) | 5 | 4 | 4 | 5 |
| Fluidization air flow rate (m3/h) | 80 | 110 | 80 | 80 |
| Agglomeration | | | | |
| Atomization air pressure (bar) | 2 | | 1.5 | 1.5 |
| Temperature of the powder bed | 70 | | 70 | 74 |
| (° C.) | | | | |
| Spraying rate for COMPRITOL ® | 42 | | 40 | 40 |
| (g/min) | | | | |
| Coating | | | | |
| Atomization air pressure (bar) | 2.5 | 3.5 | 2 | 2 |
| Temperature of the powder bed | 70 | 66 | 71 | 70 |
| (° C.) | | | | |
| Spraying rate for COMPRITOL ® | 41 | 20 | 40 | 40 |
| (g/min) | | | | |

Another example of a process for coating the drug material, thereby forming a drug-containing microparticle, involves the formation of coated microcrystals that can subsequently be incorporated into a tablet. Through selection of the appropriate polymer the microcrystals can possess diversified features such as gastroresistance and modified release due to the fact that the coated or non-coated microcrystals and microgranules preserve, after having been shaped in the form of a multiparticulate tablet, their initial properties amongst which are included masking of taste, gastroresistance and controlled release of the drug. In certain embodiments the following non-limiting list shows examples of polymers that can be selected for coating of the drug in conventional fluidized based coating equipment: ethylcellulose (EC); hydroxypropylcellulose (HPC); hydroxypropylmethylcellulose (HPMC); gelatin; gelatin/acacia; gelatin/acacia/vinvylmethylether maleic anhydride; gelatin/acacia/ethylenemaleic anhydride; carboxymethyl cellulose; polyvinvylalcohol; cellulose acetate phthalate; nitrocellulose; shellac; wax; polymethacrylate polymers such as EUDRAGIT® RS; EUDRAGIT® RL or combinations of both, EUDRAGIT® E and EUDRAGIT® NE30D; KOLLICOAT® SR30D; and mixtures thereof.

The compositions of certain embodiments can each be coated with at least one taste-masking coating. Other embodiments do not include a taste-masking coating. The taste-masking coating can mask the taste of the drug(s) in the core or the drug(s) in a coating (e.g. additional overcoat). In at least one embodiment the taste-masking coating surrounds a microparticle core to mask the taste of the drug(s) in the core. In at least one embodiment the taste-masking coating surrounds an immediate release additional overcoat to mask the taste of the drug(s) in the overcoat.

In certain embodiments the taste-masking coating formulations contain polymeric ingredients. It is contemplated that other excipients consistent with the objects of the present invention can also be used in the taste-masking coating.

In at least one embodiment, the taste-masking coating includes a polymer such as ethylcellulose, which can be used as a dry polymer (such as ETHOCEL®, Dow Corning) solubilised in organic solvent prior to use, or as an aqueous dispersion. One example of a commercially-available aqueous dispersion of ethylcellulose is AQUACOAT® (FMC Corp., Philadelphia, Pa., U.S.A.). AQUACOAT® can be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. In at least one embodiment the plasticizer is not incorporated in the pseudolatex during the manufacturing phase. Thus, prior to using the same as a coating, the AQUACOAT® is intimately mixed with a suitable plasticizer prior to use. Another aqueous dispersion of ethylcellulose is commercially available as SURELEASE® Colorcon, Inc., West Point, Pa., U.S.A.). This product can be prepared by incorporating plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which can be applied directly onto substrates.

In certain other embodiments, polymethacrylate acrylic polymers can be employed as taste-masking polymers. In at least one embodiment, the taste-masking coating is an acrylic resin lacquer used in the form of an aqueous dispersion, such as that which is commercially available from Rohm Pharma under the tradename EUDRAGIT® or from BASE under the tradename KOLLICOAT®. In certain embodiments, the acrylic coating comprises a mixture of two acrylic resin lacquers commercially available from Rohm Pharma under the tradenames EUDRAGIT® RL and EUDRAGIT® RS, respectively. EUDRAGIT® RL and EUDRAGIT® RS are copolymers of acrylic and methacrylic esters with a low content of quaternary ammonium groups, the molar ratio of ammonium groups to the remaining neutral (meth)acrylic esters being 1:20 in EUDRAGIT® RL and 1:40 in EUDRAGIT® RS. The mean molecular weight is 150,000. The code designations RL (high permeability) and RS (low permeability) refer to the permeability properties of these agents. EUDRAGIT® RL/RS mixtures are insoluble in water and in digestive fluids. However, coatings formed from the same are swellable and permeable in aqueous solutions and digestive fluids. EUDRAGIT® RL/RS dispersions or other solutions of the present invention can be mixed together in any desired ratio in order to ultimately obtain a taste-masking coating having a desirable drug dissolution profile. Non-limiting examples of formulations that can be obtained from EUDRAGIT® RL and EUDRAGIT® RS include coatings derived from 100% EUDRAGIT® RL; 50% EUDRAGIT® RL with 50% EUDRAGIT® RS; and 10% EUDRAGIT® RL with 90% EUDRAGIT® RS.

In certain other embodiments, the taste-masking polymer can be an acrylic polymer which is cationic in character based on dimethylaminoethyl methacrylate and neutral methacrylic acid esters (such as EUDRAGIT® E, commercially available from Rohm Pharma). The hydrophobic acrylic polymer coatings of certain embodiments of the present invention can further include a neutral copolymer based on poly(meth)acrylates, such as EUDRAGIT® NE (NE=neutral ester), commercially available from Rohm Pharma. EUDRAGIT® NE 30D lacquer films are insoluble in water and digestive fluids, but permeable and swellable.

In certain other embodiments, the taste-masking polymer is a dispersion of poly(ethylacrylate, methyl methacrylate) 2:1 (KOLLICOAT® EMM 30 D, BASF).

In certain other embodiments, the taste-masking polymer can be a polyvinyl acetate stabilized with polyvinylpyrrolidone and sodium lauryl sulfate such as KOLLICAOT® SR30D (BASF).

Non-limiting examples of other taste-masking polymers include hydroxypropylcellulose (HPC); hydroxypropylmethylcellulose (HPMC); hydroxyethylcellulose; gelatin; gelatin/acacia; gelatin/acacia/vinvylmethylether maleic anhydride; gelatin/acacia/ethylenemaleic anhydride; carboxymethyl cellulose; polyvinvylalcohol; nitrocellulose; polyvinylalcohol-polyethylene glycol graft-copolymers; shellac; wax; and mixtures thereof.

In certain embodiments the taste-masking coatings can be applied from one or more organic or aqueous solvent solutions or suspensions. Non-limiting examples of the organic solvent that can be used to apply the taste-masking coating include acetone, lower alcohols such as ethanol, isopropanol and alcohol/water mixtures, chlorinated hydrocarbons, and mixtures thereof. Devices used to apply the taste-masking coating include those conventionally used in pharmaceutical processing, such as fluidized bed coating devices. The taste-masking coatings can contain ingredients other than the functional polymers. One or more colorants, flavorants, sweeteners, can also be used in the taste-masking coating.

In certain embodiments a pore former can be included into the taste-masking coat in order to influence the rate of release of drug from the composition. In other embodiments, a pore former is not included in the taste-masking coat. The pore formers can be inorganic or organic, and include materials that can be dissolved, extracted or leached from the coating in the environment of use. Upon exposure to fluids in the environment of use, the pore-formers can for example be dissolved, and channels and pores are formed that fill with the environmental fluid.

For example, the pore-formers of the taste-masking coat of certain embodiments can comprise one or more water-soluble hydrophilic polymers in order to modify the release characteristics of the formulation. Non-limiting examples of hydrophilic polymers used as pore-formers in certain embodiments include hydroxypropylmethylcellulose, cellulose ethers and protein-derived materials of these polymers, the cellulose ethers, especially hydroxyalkylcelluloses and carboxyalkylcelluloses, and mixtures thereof. Also, synthetic water-soluble polymers can be used, non-limiting examples of which include polyvinylpyrrolidone, cross-linked polyvinyl-pyrrolidone, polyethylene oxide, water-soluble polydextrose, saccharides and polysaccharides, such as pullulan, dextran, sucrose, glucose, fructose, mannitol, lactose, mannose, galactose, sorbitol and mixtures thereof. In at least one embodiment that includes a hydrophilic polymer as a pore former, the taste-masking coat comprises hydroxypropyl-methylcellulose.

Other non-limiting examples of pore-formers of the taste-masking coat include alkali metal salts such as lithium carbonate, sodium chloride, sodium bromide, potassium chloride, potassium sulfate, potassium phosphate, sodium acetate, sodium citrate and mixtures thereof. The pore-forming solids can also be polymers which are soluble in the environment of use, such as Carbowaxes, and Carbopol. In addition, other possible pore-formers include diols, polyols, polyhydric alcohols, polyalkylene glycols, polyglycols, poly(a-w)alkylenediols and mixtures thereof. Non-limiting examples of other pore-formers which can be useful in formulations of certain embodiments include starch, modified starch, and starch derivatives, gums, including but not limited to xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambdacarrageenan, gum karaya, biosynthetic gum, and mixtures thereof. Other possible pore-formers include materials useful for making microporous lamina in the environment of use, non-limiting examples of which include polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous hiomopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), and mixtures thereof.

In at least one embodiment the pore-former used comprises at least one of: hypromellose substitution type 2910 with a nominal viscosity of about 6 cP, hypromellose substitution type 2906 with a nominal viscosity of about 3 cP, polyvinylpyrrolidone with a molecular weight of about 30,000, and mixtures thereof.

The amount of pore-former that can be included in the taste-masking coatings of certain embodiments can be from about 0.1% to about 80%, by weight, relative to the combined weight of polymer and pore-former. The percentage of pore former as it relates to the dry weight of the taste-masking polymer, can have an influence on the drug release properties of the composition. In at least one embodiment that uses water-soluble pore formers such as hydroxypropylmethylcellulose, a taste masking polymer:pore former dry weight ratio of between about 10:1 and about 1:1 can be present. In certain embodiments the taste masking polymer:pore former dry weight ratio is from about 8:1 to about 1.5:1; and in other embodiments from about 6:1 to about 2:1. In at least one embodiment using EUDRAGIT® NE30D as the taste masking polymer and a Hydroxypropylmethylcellulose (about 5 cps viscosity (in about a 2% aqueous solution)) such as METHOCEL® E5, PHARMACOAT® 606G as the water soluble pore former, a taste masking polymer:pore former dry weight ratio of about 2:1 is present.

Colorants that can be used in the taste-masking coating of certain embodiments include food, drug and cosmetic colors (FD&C), drug and cosmetic colors (D&C) or external drug and cosmetic colors (Ext. D&C). These colors can be dyes, lakes, and certain natural and derived colorants. Useful lakes include dyes absorbed on aluminum hydroxide or other suitable carriers.

Flavorants that can be used in the taste-masking coating of certain embodiments include natural and synthetic flavoring liquids. Non-limiting examples of such flavorants include volatile oils, synthetic flavor oils, flavoring aromatics, oils, liquids, oleoresins and extracts derived from plants, leaves, flowers, fruits, stems and combinations thereof. A non-limiting representative list of these includes citric oils, such as lemon, orange, grape, lime and grapefruit, and fruit essences, including apple, pear, peach, grape, strawberry, raspberry, cherry, plum, pineapple, apricot, or other fruit flavors. Other useful flavorants include aldehydes and esters, such as benzaldehyde (cherry, almond); citral, i.e., alpha-citral (lemon, lime); neral, i.e., beta-citral (lemon, lime); decanal (orange, lemon); aldehyde C-8 (citrus fruits); aldehyde C-9 (citrus fruits); aldehyde C-12 (citrus fruits); tolyl aldehyde (cherry, almond); 2,6-dimethyloctanal (green fruit); 2-dodenal (citrus mandarin); and mixtures thereof.

Non-limiting examples of sweeteners that can be used in the taste-masking coating of certain embodiments include glucose (corn syrup), dextrose, invert sugar, fructose (when not used as a carrier); saccharin and its various salts (such as sodium salt); dipeptide sweeteners such as aspartame; dihydrochalcone compounds, glycyrrhizin; Steva Rebaudiana (Stevioside); chloro derivatives or sucrose such as sucralose; sugar alcohols such as sorbitol, mannitol, and xylitol; and mixtures thereof. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners can be used alone or in any combination thereof.

The taste-masking coating of certain embodiments can also include one or more pharmaceutically acceptable excipients such as lubricants, emulsifiers, anti-foaming agents, plasticisers, and solvents.

Lubricants can be included to help reduce friction of cores (e.g. microparticle cores) during manufacturing. Non-limiting examples of the lubricants that can be used in the taste masking coat of certain embodiments include adipic acid, magnesium stearate, calcium stearate, zinc stearate, calcium silicate, magnesium silicate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil, waxy fatty acid esters such as glyceryl behenate, (i.e. COMPRITOL™), STEAR-O-WET™, MYVATEX™ TL and mixtures thereof. In at least one embodiment, a lubricant is selected from magnesium stearate, talc and mixtures thereof. The lubricant(s) can be present in an amount of from about 1% to about 100% by weight, for example about 5% by weight, about 10% by weight, about 20% by weight, about 30% by weight, about 40% by weight, about 50% by weight, about 60% by weight, about 70% by weight, about 80% by weight, or about 90% by weight of the polymer dry weight in the taste-masking coat. For example, in certain embodiments wherein the taste masking polymer is EUDRAGIT® NE30D or EUDRAGIT® NE40D (Rohm America LLC) together with a hydrophilic pore former, the lubricant is present in an amount of from about 1% to about 30% by weight, for example about 5% by weight, about 10% by weight, about 15% by weight, about 20% by weight, or about 25% by weight of the polymer dry weight; in other embodiments from about 2% to about 20%; and in still other embodiments at about 10% by weight of the taste-masking coat dry weight. In other embodiments where the taste masking polymer is ethylcellulose (ETHOCEL™ PR100, PR45, PR20, PR10 or PR7 polymer, or a mixture thereof), the lubricant can be present in an amount of from about 10% to about 100%, for example about 10% by weight, about 20% by weight, about 30% by weight, about 40% by weight, about 50% by weight, about 60% by weight, about 70% by weight, about 80% by weight, or about 90% by weight of the taste masking coat dry weight; in another embodiment from about 20% to about 80%; and in still another embodiments at about 50% by weight of the taste masking coat dry weight. In other embodiments, the taste-masking coat does not include a pore former.

Emulsifying agent(s) (also called emulsifiers or emulgents) can be included in the taste-masking coat to facilitate actual emulsification during manufacture of the coat, and also to increase emulsion stability during the shelf-life of the product. Emulsifying agents useful for the taste-masking coat composition of certain embodiments include, but are not limited to naturally occurring materials and their semi synthetic derivatives, such as the polysaccharides, as well as glycerol esters, cellulose ethers, sorbitan esters (e.g. sorbitan monooleate or SPAN™ 80), and polysorbates (e.g. TWEEN™ 80). Combinations of emulsifying agents are operable. In at least one embodiment, the emulsifying agent is TWEEN™ 80. The emulsifying agent(s) can be present in an amount of from about 0.01% to about 5% by weight of the taste-masking polymer dry weight. For example, in certain embodiments the emulsifying agent is present in an amount of from about 0.05% to about 3%; in other embodiments from about 0.08% to about 1.5%, and in still other embodiments at about 0.1% by weight of the taste-masking polymer dry weight.

Anti-foaming agent(s) can be included in the taste-masking coat to reduce frothing or foaming during manufacture of the coat. Anti-foaming agents useful for the coat composition of certain embodiments include, but are not limited to simethicone, polyglycol, silicon oil, and mixtures thereof. In at least one embodiment the anti-foaming agent is Simethicone C. The anti-foaming agent can be present in an amount of from about 0.1% to about 10%, for example about 0.5%, about 1.0%, about 2%, about 3%, about 4%, about 5%, about 6%, about 7%, about 8%, or about 9% of the taste-masking coat dry weight. For example, in certain embodiments the anti-foaming agent is present in an amount of from about 0.2% to about 5%; in other embodiments from about 0.3% to about 1%, and in still other embodiments at about 0.6% by weight of the taste-masking polymer dry weight.

Plasticizer(s) can be included in the taste-masking coat to provide increased flexibility and durability during manufacturing. Non-limiting examples of plasticisers that can be used in the taste-masking coat of certain embodiments include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, and mixtures thereof. The plasticizer(s) can be present in an amount of from about 1% to about 80% of the taste-masking polymer dry weight. For example, in certain embodiments the plasticizer is present in an amount of from about 5% to about 50%, in other embodiments from about 10% to about 40%, and in still other embodiments at about 20% of the taste-masking polymer dry weight.

In certain embodiments where the taste-masking coating surrounds a microparticle, the taste-masking coating can be present in an amount of from about 1% to about 90% by weight, for example about 10% by weight, about 20% by weight, about 30% by weight, about 40% by weight, about 50% by weight, about 60% by weight, about 70% by weight, or about 80% by weight of the microparticle, depending upon the choice of polymer, the ratio of polymer:pore former (in certain embodiments where a pore former is present), and the total surface area of the microparticle formulation. Since a certain thickness of taste-masking coating has to be achieved in order to achieve effective taste masking, the amount of taste-masking polymer coating used during manufacture is related to the total surface area of the batch of uncoated microparticles that requires a coating. The taste-masking polymer surface area coverage can range from about 0.5 mg/cm² to about 20 mg/cm². For example, in certain embodiments the surface area coverage of the taste-masking polymer is from about 0.6 mg/cm² to about 10 mg/cm², and in other embodiments is from about 1 mg/cm² to about 5 mg/cm². In at least one embodiment of the invention, EUDRAGIT® E is employed as the taste-masking polymer at a surface area coverage of about 4 mg/cm². One approach in estimating the total surface area of a multiparticulate batch is the permeability method according to Blaine (ASTM Des. C 205-55), which is based upon the mathematical model of laminar flow through capillaries arranged in parallel.

In the absence of an accurate determination of total surface area of a microparticle, the amount of taste-masking polymer to be applied can be expressed as a percentage of the uncoated microparticle. For example, in certain embodiments the taste-masking coating is present in an amount of from about 5% to about 60%; in other embodiments from about 10% to about 40%; and in still other embodiments from about 15% to about 30% by weight of the microparticle. In at least one embodiment the taste-masking coating is present in an amount of about 25% by weight of the microparticle.

The diameter of the microparticles (with or without the taste-masking coating) can range from about 50 μm to 800 μm. For example, in certain embodiments the diameter of the microparticles range from about 100 μm to about 600 μm; in other embodiments from about 150 μm to about 500 μm; in still other embodiments from about 200 μm to about 300 μm; and in even still other embodiments from about 200 μm to about 250 μm.

Particle size evaluation of microparticles with a diameter range of approx 50 micron to approx 800 micron can be assessed in accordance with USP method 786 using US mesh numbers 20 and 270.

An osmotic subcoat partially or fully surrounds the core of the composition. In at least one embodiment the core is a microparticle core. The osmotic subcoat includes at least one osmotic deposition vehicle and at least one osmotic agent in an amount sufficient to achieve an osmotic pressure gradient across one or more outer membrane(s)/coating(s) for the transport of solvent (e.g. water) from the external environment of use into the core, and the transport of drug from the core into the external environment of use, which can lead to an increased release of the drug from the composition. In certain embodiments the outer membrane/coating is a modified release overcoat that surrounds the osmotic subcoat.

In certain embodiments the osmotic subcoat provides increased release of the drug from the core of the composition. In at least one embodiment the osmotic subcoat provides substantially full release of the drug from the core. In certain embodiments it is contemplated that the osmotic subcoat is able to provide increased stability to the composition.

The osmotic agent can be any ingredient that can generate an osmotic pressure gradient for the transport of drug solution from the core of the composition out to the external environment of use. The osmotic agent is soluble in aqueous and biological fluids. Non-limiting examples of osmotic agents include ionizing compounds, polar compounds, inorganic acids, organic acids, bases, salts and mixtures thereof. In at least one embodiment, the osmotic agent is a solid and dissolves to form a solution with fluids imbibed into the osmotic subcoat. A wide variety of osmotic agents can be used to provide the osmotic pressure gradient used to drive the drug from the microparticle core. Non-limiting examples of inorganic salts useful as osmotic agents include lithium chloride, lithium sulfate, lithium phosphate, magnesium chloride, magnesium sulfate, potassium chloride, potassium sulfate, potassium phosphate, potassium acid phosphate, sodium chloride, sodium sulfate, sodium phosphate, sodium sulfite, sodium nitrate, sodium nitrite, and mixtures thereof. Non-limiting examples of salts of organic acids useful as osmotic agents include sodium citrate, potassium acid tartrate, potassium bitartrate, sodium bitartrate, and mixtures thereof. Non-limiting examples of ionizable solid acids useful as osmotic agents include tartaric, citric, maliec, malic, fumaric, tartronic, itaconic, adipic, succinic, mesaconic acid, and mixtures thereof. Non-limiting examples of other compounds useful as osmotic agents include potassium carbonate, sodium carbonate, ammonium carbonate, calcium lactate, mannitol, urea, inositol, magnesium succinate, sorbitol, and carbohydrates such as raffinose, sucrose, glucose, lactose, lactose monohydrate, and a blend of fructose glucose. Combinations of these compounds are permissible. In at least one embodiment the osmotic agent includes sodium chloride, sodium bromide, sodium bisulfate, sodium citrate, potassium acid tartrate, citric acid, fumaric acid, mannitol, sucrose, or a mixture thereof. In at least one embodiment, the osmotic agent is sodium chloride. The osmotic agent can be present in an amount of from about 1% to about 99%, for example about 5%, about 10%, about 20%, about 30%, about 40%, about 50%, about 60%, about 70%, about 80%, or about 90% of the osmotic subcoat dry weight. For example, in certain embodiments the osmotic agent is present in an amount of from about 1% to about 66%, in other embodiments from about 1% to about 40%, in still other embodiments from about 25% to about 35%, and in even still other embodiments at about 33% of the osmotic subcoat dry weight.

The osmotic agent, being located in the osmotic subcoat, is separated from the drug located in the core of certain embodiments. In certain embodiments the osmotic agent is separated from both the drug located in the core, and the drug located in an additional overcoat. The osmotic subcoat provides for the substantial separation of the drug(s) from the osmotic agent(s), and the separation of the drug(s) from the polymer(s) and other excipients of the modified release overcoat, into separate compartments/layers. Without wishing to be bound to any particular theory, it is believed that in certain embodiments this separation can reduce unfavorable interactions between the drug(s) and the osmotic agent(s), and between the drug(s) and the polymer(s) and other excipients of the modified release overcoat. For example, the osmotic agent of certain embodiments can be generally hygroscopic in nature, and can tend to attract water that can lead to the degradation of nearby components. In certain embodiments where the osmotic agent is substantially separated from the drug, the drug can be less prone to degradation from the water drawn in by the osmotic agent. In certain embodiments unfavorable interactions between the drug(s) and the osmotic agent(s), and between the drug(s) and the polymer(s) and other excipients of the modified release overcoat can be reduced. In at least one embodiment the osmotic agent(s) and/or the polymer(s) and other excipients of the modified release overcoat do not substantially interact with and/or do not substantially affect the solubility of the drug(s).

The osmotic deposition vehicle functions as a carrier for the osmotic agent. In certain embodiments the osmotic deposition vehicle does not substantially affect the drug release. In certain embodiments the osmotic deposition vehicle does not act as a diffusion barrier to the release of the drug. In at least one embodiment the release profile of the osmotic agent is substantially the same as the release profile of the drug.

The osmotic deposition vehicle of certain embodiments can be any type of hydrophilic polymer. Non-limiting examples of polymers that can be used as the osmotic deposition vehicle include polyvinyl pyrrolidone, hydroxyethyl cellulose, hydroxypropyl cellulose, low molecular weight hydroxypropyl methylcellulose (HPMC), polymethacrylate, ethyl cellulose, and mixtures thereof. In at least one embodiment, the osmotic deposition vehicle is a low molecular weight and a low viscosity hydrophilic polymer. A wide variety of low molecular weight and low viscosity hydrophilic polymers can act as the osmotic deposition vehicle. In at least one embodiment the hydrophilic polymer used as the osmotic deposition vehicle is a low molecular weight hydroxypropylmethylcellulose (HPMC) polymer. Non-limiting examples of HPMC polymers that can be used as the osmotic deposition vehicle include PHARMACOAT® 606, PHARMACOAT® 606G, PHARMACOAT® 603, METHOCEL® E3, METHOCEL® E5, METHOCEL® E6, and mixtures thereof. Further non-limiting examples of polymers that can be used as the osmotic deposition vehicle include hypromellose substitution type 2910 with a nominal viscosity of about 6 cP, hypromellose substitution type 2906 with a nominal viscosity of about 3 cP, polyvinylpyrrolidone with a molecular weight of about 30,000, and mixtures thereof. The osmotic deposition vehicle can be present in an amount of from about 1% to about 99%, for example about 5%, about 10%, about 20%, about 30%, about 40%, about 50%, about 60%, about 70%, about 80%, or about 90% of the osmotic subcoat dry weight. For example, in certain embodiments the osmotic deposition vehicle can be present in an amount from about 20% to about 80%, in other embodiments from about 30% to about 75%, in still other embodiments from about 60% to about 70%, and in even still other embodiments at about 66% of the osmotic subcoat dry weight.

The osmotic pressure gradient and rate of release of the drug can be controlled in certain embodiments by varying the level of the osmotic agent and/or the level of the osmotic deposition vehicle in the osmotic subcoat. Certain embodiments do not require a sealcoat around the core to control the osmotic effect. In at least one embodiment, the osmotic dosage form does not include a sealcoat.

It is contemplated that in alternative embodiments, other excipients consistent with the objects of the present invention can also be used in the osmotic subcoat.

In at least one embodiment, the osmotic subcoat includes about 66.67% PHARMACOAT® 606, and about 33.33% sodium chloride by weight of the osmotic subcoat dry weight.

In certain embodiments, the osmotic deposition vehicle is present in an amount of from about 1% to about 30%, preferably from about 1% to about 20%, and more preferably from about 3% to about 10% of the osmotic subcoat formulation. In such embodiments, the osmotic agent is present in an amount of from about 0.1% to about 50%, preferably from about 0.1% to about 20%, and more preferably from about 0.5% to about 5% of the osmotic subcoat formulation. In such embodiments, water is present in an amount of from about 20% to about 98.9% of the osmotic subcoat formulation. In at least one embodiment, the osmotic subcoat formulation includes about 7% osmotic deposition vehicle; about 1.05% osmotic agent; and about 91.95% water. In at least one embodiment the osmotic deposition vehicle is PHARMACOAT® 606, and the osmotic agent is NaCl.

In certain embodiments the weight gain from the osmotic subcoat over the uncoated microparticle core is from about 0.5% to about 20%, preferably from about 1% to about 10%, and more preferably from about 3% to about 7%. In at least one embodiment the weight gain from the osmotic subcoat over the uncoated microparticle core is about 4.6%.

In certain embodiments the manufacturing process for the osmotic subcoat can be as follows: At least one osmotic agent is dissolved in water. In at least one embodiment the osmotic agent is NaCl. The solution of osmotic agent and water is then heated to about 60° C. The osmotic deposition vehicle is then added gradually to the solution containing the osmotic agent and water. In at least one embodiment the osmotic deposition vehicle is PHARMACOAT® 606. A magnetic stirrer can be used to aid in the mixing of the osmotic deposition vehicle to the solution of osmotic agent and water. The resultant osmotic subcoating solution can then be used to coat the microparticles in a fluidized bed granulator, such as a granulator manufactured by GLATT® (Germany) or AEROMATIC® (Switzerland). In certain embodiments the microparticles are coated with the osmotic subcoating solution in a GLATT®-Powder-Coater-Granulator (GPCG 1.1) to about a 4.6% weight gain with the following parameters: An inlet temperature of from about 10° C. to about 70° C., (for example in certain embodiments an inlet temperature from about 30° C. to about 55° C., and in other embodiments from about 40° C. to about 45° C.); an outlet temperature of from about 10° C. to about 70° C., (for example in certain embodiments an outlet temperature from about 20° C. to about 45° C., and in other embodiments from about 30° C. to about 35° C.); a product temperature of from about 10° C. to about 70° C., (for example in certain embodiments a product temperature from about 20° C. to about 45° C., and in other embodiments from about 30° C. to about 35° C.); an air flow of from about 10 c.m/h to about 180 c.m/h, (for example in certain embodiments an air flow from about 40 c.m/h to about 120 c.m/h, and in other embodiments from about 60 c.m/h to about 80 c.m/h); an atomizing pressure of from about 0.5 bar to about 4.5 bar, (for example in certain embodiments an atomizing pressure from about 1 bar to about 3 bar, and in other embodiments about 2 bar); a curing temperature of from about 10° C. to about 70° C., (for example in certain embodiments a curing temperature from about 20° C. to about 50° C., and in other embodiments from about 30° C. to about 40° C.); and a curing time of from about 5 minutes to about 720 minutes; (for example in certain embodiments a curing time from about 10 minutes to about 120 minutes, and in other embodiments about 30 minutes). In at least one embodiment the microparticles are coated with the osmotic subcoating solution in a GLATT®-Powder-Coater-Granulator (GPCG 1.1) to about a 4.6% weight gain with the following parameters: An inlet temperature of from about 40° C. to about 45° C.; an outlet temperature of from about 30° C. to about 35° C.; a product temperature of from about 30° C. to about 35° C.; an air flow of from about 60 c.m/h to about 80 c.m/h; an atomizing pressure of about 2 bar; a curing temperature of from about 30° C. to about 40° C.; and a curing time of about 30 minutes.

In certain other embodiments, the manufacturing process for the osmotic subcoat can for example be as follows: The osmotic agent and osmotic deposition vehicle are coated onto the drug-containing microparticle cores by a dry powder coating technique. Micronised sodium chloride and an aqueous 10% w/w HPMC 6 cps binder solution are fed through separate inlets onto drug-containing microparticle cores in a fluidised bed coater (GLATT® GPCG-1, Wurster insert), with the following parameters: An inlet air temperature of from about 55° C. to about 60° C.; a product temperature of from about 45° C. to about 47° C.; an outlet air temperature of from about 40° C. to about 41° C.; an air flow rate of from about 60 c.m/h to about 80 c.m/h; an atomizing air pressure of from about 1 bar to about 1.5 bar; a powder feed rate of from about 10 g/min to about 12 g/min; and a spray rate of from about 3 g/min to about 5 g/min. Any other manufacturing process and/or technology resulting in a coating formulation of the osmotic subcoat as described herein and consistent with the objects of the invention can also be used.

The core of the composition is further coated with at least one modified release overcoat. As used herein, the term “modified release overcoat” refers to the functional coat comprising at least one modified release polymer, that at least partially or fully_surrounds each osmotic subcoated core. In at least one embodiment the modified release overcoat surrounds an osmotic subcoated microparticle. As used herein, the terms “overcoat” and “coat” are used interchangeably when referring to the modified release overcoat. The modified release overcoat is designed to achieve a modified release of the drug from the core. For example, the modified release overcoat can be an enteric coat with low solubility at a gastric pH to reduce or minimize the drug release in the lumen of the stomach, while possessing pH-dependent solubility to facilitate drug release in the duodenum.

In certain embodiments, the modified release overcoat can be a delayed release coating that provides a delayed release of the drug with a predetermined lagtime that is independent of, or alternatively dependent on, the pH of the dissolution medium. For example, by increasing the thickness of the modified release overcoat using a pH independent diffusion polymer, lagtimes of about 1 hour, about 2 hours, about 3 hours, about 4 hours, about 5 hours, about 6 hours, about 7 hours, about 8 hours, about 9 hours, about 10 hours, about 11 hours, or about 12 hours can be achieved. In addition, in certain embodiments the modified release overcoat can include modified release polymers that become soluble above a certain pH, which could provide a further delay in drug release. Drug release from such a system can be reduced or delayed until the critical pH for the polymer of choice is reached. With either approach, following the predetermined lag, the drug is released, for example within about 1 hour for an immediate release pulse, or alternatively over a prolonged period of time, for example from about 3 to about 24 hours for other delayed release profiles.

In other embodiments, the modified release overcoat can provide a diffusion barrier that is independent of pH, thus facilitating a modified release profile, with substantially full release of the drug occurring in from about 3 to at least about 24 hours, for example about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, or about 23 hours. In at least one embodiment, the modified release overcoat provides a delayed and extended release of the drug from the core with substantially full release in about 24 hours. In certain embodiments the modified release overcoat provides a modified release of the drug from the core and substantially full release of the drug being achieved in, for example, about 16 hours, about 17 hours, about 18 hours, about 19 hours, about 20 hours, about 21 hours, about 22 hours, about 23 hours or about 24 hours. In certain embodiments full release of the drug is achieved in more than about 24 hours. In at least one embodiment the modified release overcoat provides modified release of the drug from a microparticle core, and substantially full release of the drug being achieved in about 24 hours.

In certain embodiments, the modified release overcoat can provide substantially full release of the drug from the core without requiring the use of any pore formers. In at least one embodiment, the modified release overcoat does not include any pore formers. Non-limiting examples of unnecessary pore formers that are not required in the modified release overcoat of these embodiments include alkali metal salts, alkaline earth metal salts, saccharides, aliphatic polyols, aromatic polyols, polyethylene glycol, sorbitol, glucose, hydrophilic polymers such as hydroxypropyl methylcellulose, and mixtures thereof.

The modified release overcoat of certain embodiments includes at least one modified release polymer in an amount sufficient to achieve a modified release of the drug. In at least one embodiment of the invention the modified release polymer is an acrylic polymer. Non-limiting examples of acrylic polymers include acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid, methacrylic acid alkylamine copolymer, poly(methyl methacrylate), poly(methacrylic acid) (anhydride), glycidyl methacrylate copolymers, and mixtures thereof.

In at least one embodiment the modified release overcoat comprises polymerizable quaternary ammonium compounds, of which non-limiting examples include quaternized aminoalkyl esters and aminoalkyl amides of acrylic acid and methacrylic acid, for example β-methacryl-oxyethyl-trimethyl-ammonium methosulfate, β-acryloxy-propyl-trimethyl-ammonium chloride, and trimethylaminomethyl-methacrylamide methosulfate, and mixtures thereof. The quaternary ammonium atom can also be part of a heterocycle, as in methacryloxyethylmethyl-morpholiniom chloride or the corresponding piperidinium salt, or it can be joined to an acrylic acid group or a methacrylic acid group by way of a group containing hetero atoms, such as a polyglycol ether group. Further non-limiting examples of polymerizable quaternary ammonium compounds include quaternized vinyl-substituted nitrogen heterocycles such as methyl-vinyl pyridinium salts, vinyl esters of quaternized amino carboxylic acids, styryltrialkyl ammonium salts, and mixtures thereof. Other polymerizable quaternary ammonium compounds useful in the present invention include acryl- and methacryl-oxyethyltrimethyl-ammonium chloride and methosulfate, benzyldimethylammoniumethyl-methacrylate chloride, diethylmethylammoniumethyl-acrylate and -methacrylate methosulfate, N-trimethylammoniumpropylmethacrylamide chloride, N-trimethylammonium-2,2-dimethylpropyl-1-methacrylate chloride, and mixtures thereof.

In certain embodiments the modified release polymer(s) of the modified release overcoat includes an acrylic polymer comprised of one or more ammonio methacrylate copolymers. Ammonio methacrylate copolymers (such as those sold under the Trade Mark EUDRAGIT® RS and RL) are described in NF XVII as fully polymerized copolymers of acrylic and methacrylic acid esters with a low content of quaternary ammonium groups. In at least one embodiment the modified release overcoat includes two or more ammonio methacrylate copolymers having differing physical properties. In such embodiments, the permeability properties of the modified release overcoat can be modified and a desired dissolution profile achieved, for example, by changing the molar ratio of the quaternary ammonium groups to the neutral (meth)acrylic esters.

In certain embodiments the acrylic polymer(s) of the modified release overcoat can include a polymer whose permeability is pH dependent. Non-limiting examples of such polymers include anionic polymers synthesized from methacrylic acid and methacrylic acid methyl ester. Such polymers are commercially available, e.g., from Rohm Pharma GmbH under the tradename EUDRAGIT® L and EUDRAGIT® S. The ratio of free carboxyl groups to the esters is known to be about 1:1 in EUDRAGIT®L and about 1:2 in EUDRAGIT®S. EUDRAGIT® L is insoluble in acids and pure water, but becomes increasingly permeable above about pH 5.0. EUDRAGIT® S is similar, except that it becomes increasingly permeable above about pH 7. In certain embodiments where the modified release overcoat is hydrophobic, the overcoat can also include a polymer which is cationic in character based on dimethylaminoethyl methacrylate and neutral methacrylic acid esters (such as EUDRAGIT® E, commercially available from Rohm Pharma). In addition the modified release overcoat of certain embodiments can include a neutral copolymer based on poly(meth)acrylates, such as EUDRAGIT® NE 30D (NE=neutral ester), commercially available from Rohm Pharma. EUDRAGIT® NE 30D lacquer films are insoluble in water and digestive fluids, but permeable and swellable.

In certain embodiments the modified release polymer(s) in the modified release overcoat include a dispersion of poly(ethylacrylate, methyl methacrylate) 2:1 (KOLLICOAT® EMM 30 D, BASF). In certain embodiments the modified release polymer can be a polyvinyl acetate stabilized with polyvinylpyrrolidone and sodium lauryl sulfate such as KOLLICOAT® SR30D (BASF). The dissolution profile of such embodiments can be altered by changing the relative amounts of different acrylic resin lacquers included in the coating. Also, by changing the molar ratio of polymerizable permeability-enhancing agent (e.g., the quaternary ammonium compounds) to the neutral (meth)acrylic esters, the permeability properties (and thus the dissolution profile) of the modified release overcoat of certain embodiments can be modified.

In at least one embodiment the modified release polymer is ethylcellulose, which can be used as a dry polmer (such as ETHOCEL®, Dow Corning) solubilised in organic solvent prior to use, or as an aqueous dispersion. One example of a commercially available aqueous dispersion of ethylcellulose is AQUACOAT® (FMC Corp., Philadelphia, Pa., U.S.A.). AQUACOAT® can be prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, the AQUACOAT® is intimately mixed with a suitable plasticizer prior to use. Another aqueous dispersion of ethylcellulose is commercially available as SURELEASE® (Colorcon, Inc., West Point, Pa., U.S.A.). This product can be prepared by incorporating a plasticizer into the dispersion during the manufacturing process. A hot melt of a polymer, plasticizer (e.g. dibutyl sebacate), and stabilizer (e.g. oleic acid) is prepared as a homogeneous mixture, which is then diluted with an alkaline solution to obtain an aqueous dispersion which can be applied directly onto substrates.

Other non-limiting examples of modified release polymers that can be used in the modified release overcoat include cellulose acetate phthalate, cellulose acetate trimaletate, hydroxy propyl methylcellulose phthalate, polyvinyl acetate phthalate, polyvinyl alcohol phthalate, polyvinyl acetaldiethylamino acetate, hydroxypropyl methylcellulose acetate succinate, shellac, hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, ethyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, pullulan, collagen, casein, agar, gum arabic, sodium carboxymethyl cellulose, (swellable hydrophilic polymers) poly(hydroxyalkyl methacrylate) (molecular weight from about 5 k to about 5000 k), polyvinylpyrrolidone (molecular weight from about 10 k to about 360 k), anionic and cationic hydrogels, zein, polyamides, polyvinyl alcohol having a low acetate residual, a swellable mixture of agar and carboxymethyl cellulose, copolymers of maleic anhydride and styrene, ethylene, propylene or isobutylene, pectin (molecular weight from about 30 k to about 300 k), polysaccharides such as agar, acacia, karaya, tragacanth, algins and guar, polyacrylamides, POLYOX® polyethylene oxides (molecular weight from about 100 k to about 5000 k), AQUAKEEP® acrylate polymers, diesters of polyglucan, crosslinked polyvinyl alcohol and poly N-vinyl-2-pyrrolidone, sodium starch glycolate (e.g. EXPLOTAB®; Edward Mandell C. Ltd.), hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. POLYOX®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. EUDRAGIT®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan, and mixtures thereof.

In at least one embodiment, the polymer(s) used in the modified release overcoat include SURELEASE®. The SURELEASE® polymer can be present in an amount of from about 5% to about 100% by weight, for example about 10% by weight, about 20% by weight, about 30% by weight, about 40% by weight, about 50% by weight, about 60% by weight, about 70% by weight, about 80% by weight, or about 90% by weight of the modified release overcoat dry weight, depending on the drug used and the modified release profile desired. For example, in certain embodiments the SURELEASE® polymer can be present in an amount of from about 20% to about 100%; and in other embodiments from about 40% to about 90% of the modified release overcoat dry weight. In at least one embodiment, the SURELEASE® polymer is present in an amount of about 100% of the modified release overcoat dry weight.

In certain embodiments, the polymer(s) used in the modified release overcoat include one or more acrylate dispersions such as EUDRAGIT® NE30D, EUDRAGIT® NE40D (Rohm America LLC), KOLLICOAT® SR 30D, and SURELEASE®. Combinations of these polymers is permissible. In at least one embodiment, the modified release polymer(s) used in the modified release overcoat include EUDRAGIT® NE30D. In such embodiments the polymer(s) can be present in an amount of from about 5% to about 99% by weight of the modified release overcoat dry weight, depending on the drug used and the modified release profile desired. For example, in certain of these embodiments the polymer(s) can be present in an amount of from about 20% to about 99%; in other embodiments from about 50% to about 95%, and in still other embodiments from about 60% to about 90% of the modified release overcoat dry weight. In at least one of such embodiments, the polymer(s) is present in an amount of about 75%, and in at least one other embodiment the polymer(s) is present in an amount of about 87.6% of the modified release overcoat dry weight.

In certain embodiments the modified release overcoat comprises polymers that can facilitate mucoadhesion within the gastrointestinal tract. Non-limiting examples of polymers that can be used for mucoadhesion include carboxymethylcellulose, polyacrylic acid, CARBOPOL™, POLYCARBOPHIL™, gelatin, and other natural or synthetic polymers. Combinations of such polymers are operable.

In at least one embodiment the modified release overcoat comprises at least one of the following types of polymers: poly(meth)acrylates neutral copolymer aqueous dispersions, polyvinyl acetate aqueous dispersions, ethylcellulose from which a 5% solution in a blend of about 80% toluene/about 25% ethanol has a viscosity of from about 50 cP to about 100 cP, dispersions of poly(ethylacrylate, methyl acrylate), and mixtures thereof.

In at least one embodiment of the modified release overcoat comprises at least one of the following: a poly(meth)acrylate neutral copolymer, a polyvinyl acetate, an ethylcellulose, a poly(ethyl acrylate, methyl acrylate), and combinations thereof.

In certain embodiments one or more pharmaceutically acceptable excipients consistent with the objects of the present invention can be used in the modified release overcoat, such as a lubricant, an emulsifier, an anti-foaming agent, a plasticiser, and/or a solvent.

In certain embodiments the modified release overcoat comprises:
(i) at least one film-forming polymer which is insoluble in the liquids of the digestive tract, present in an amount of from about 50% to about 90% (e.g. in at least one embodiment present in an amount of from about 50% to about 80%, or about 70% or about 60%) by weight of dry matter of the modified release overcoat composition, and including at least one non-hydrosoluble cellulose derivate, (e.g. ethylcellulose, cellulose acetate, or a mixture thereof);
(ii) at least one nitrogen-containing polymer, present in an amount of from about 2% to about 25% (e.g. in at least one embodiment present in an amount of from about 5% to about 15%, or about 20%) by weight of dry matter of the modified release overcoat composition, and including at least one polyacrylamide, poly-N-vinylaride, poly-N-vinyl-lactame, polyvinylpyrrolidone, or a mixture thereof;
(iii) at least one plasticizer present in an amount of from about 2% to about 20% (e.g. in at least one embodiment present in an amount of from about 4% to about 15%) by weight of dry matter of the modified release overcoat composition, and including at least one of the following compounds: glycerol esters, phtalates, citrates, sebacates, cetylalcohol esters, castor oil, cutin, or a mixture thereof;
(iv) at least one surface-active and/or lubricating agent, present in an amount of from about 2% to about 20% (e.g. in at least one embodiment present in an amount of from about 4% to about 15%) by weight of dry matter of the modified release overcoat composition, and chosen from anionic surfactants such as the alkali metal and alkaline-earth metal salts of fatty acids, (e.g. stearic acid, oleic acid, and mixtures thereof), and/or from nonionic surfactants such as polyoxyethylenated esters of sorbitan, polyoxyethylenated esters of sorbitan, polyoxyethylenated derivatives of castor oil, and/or from lubricants such as stearates (e.g. calcium, magnesium, aluminium, zinc stearate and mixtures thereof), stearylfumarates (e.g. sodium stearylfumarate, glyceryl behenate and mixtures thereof); and mixtures thereof;
   wherein the modified release overcoated composition can remain in the small intestine for a period of at least about 5 hours. For example in certain embodiments the modified release overcoated composition can remain in the small intestine for at least about 7 hours; and in other embodiments for a period of between about 8 hours and about 24 hours, for example about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, or about 23 hours; so as to allow absorption of the drug during at least part of its time in the small intestine.

In a prophetic example of at least one embodiment of the invention, microparticles are coated with a modified release overcoat in a fluidized bead coater with the following coating solution:

| | |
|---|---|
| Ethylcellulose | 44.7 g |
| PVP | 4.8 g |
| Castor oil | 4.8 g |
| Magnesium Stearate | 6.1 g |
| Acetone | 479 g |
| Isopranol | 53 g |

In certain embodiments, the release of the drug from a modified release formulation can be further influenced (i.e., adjusted to a desired rate) by the addition of one or more pore-formers to the modified release overcoat. In certain other embodiments the modified release overcoat does not include a pore-former. The pore-former(s) can be inorganic or organic, and can include materials that can be dissolved, extracted or leached from the modified release overcoat in the environment of use. Upon exposure to fluids in the environment of use, the pore-former(s) are, for example, dissolved, and channels and pores are formed that fill with the environmental fluid. For example, the pore-formers can include one or more water-soluble hydrophilic polymers in order to modify the release characteristics of the formulation. Non-limiting examples of suitable hydrophilic polymers include hydroxypropylmethylcellulose, cellulose ethers and protein-derived materials of these polymers, the cellulose ethers, (e.g. hydroxyalkylcelluloses and carboxyalkylcelluloses), and mixtures thereof. Also, synthetic water-soluble polymers can be used, such as polyvinylpyrrolidone, cross-linked polyvinyl-pyrrolidone, polyethylene oxide, water-soluble polydextrose, saccharides and polysaccharides, such as pullulan, dextran, sucrose, glucose, fructose, mannitol, lactose, mannose, galactose, sorbitol, and mixtures thereof. In at least one embodiment the hydrophilic polymer(s) used as pore-formers include hydroxypropyl-methylcellulose. Other examples of pore-formers include alkali metal salts such as lithium carbonate, sodium chloride, sodium bromide, potassium chloride, potassium sulfate, potassium phosphate, sodium acetate, sodium citrate, and mixtures thereof. The pore-forming solids can also be polymers which are soluble in the environment of use, such as CARBOWAXES®, CARBOPOL®, and the like. The pore-formers can include diols, polyols, polyhydric alcohols, polyalkylene glycols, polyglycols, poly(a-w)alkylenediols, and mixtures thereof. Other pore-formers which can be useful in other embodiments include starch, modified starch, and starch derivatives, gums, including but not limited to xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambdacarrageenan, gum karaya, biosynthetic gum, and mixtures thereof. Other pore-formers include materials useful for making microporous lamina in the environment of use, such as polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous hiomopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), and mixtures thereof.

In at least one embodiment the pore-former used comprises at least one of: hypromellose substitution type 2910 with a nominal viscosity of about 6 cP, hypromellose substitution type 2906 with a nominal viscosity of about 3 cP, polyvinylpyrrolidone with a molecular weight of about 30,000, and mixtures thereof.

The amount of pore-former that can be included in the modified release overcoats of certain embodiments can be from about 0.1% to about 80%, for example about 10%, about 20%, about 30%, about 40%, about 50%, about 60%, or about 70%, by weight, relative to the combined weight of polymer and pore-former. The percentage of pore former as it relates to the dry weight of the modified release polymer, can have an influence on the drug release properties of the composition. In at least one embodiment that uses water-soluble pore formers such as hydroxypropylmethylcellulose, a modified release polymer:pore former dry weight ratio of between about 10:1 and about 1:1 can be present. In certain embodiments the modified release polymer:pore former dry weight ratio is from about 8:1 to about 1.5:1; and in other embodiments from about 6:1 to about 2:1. In at least one embodiment using EUDRAGIT® NE30D as the modified release polymer and a Hydroxypropylmethylcellulose (about 5 cps viscosity (in about a 2% aqueous solution)) such as METHOCEL® E5, PHARMACOAT® 606G as the water-soluble pore former, a modified release polymer:pore former dry weight ratio of about 2:1 is present.

The modified release overcoat of certain embodiments can include one or more swelling agents (i.e., a pharmaceutically acceptable agent provided in an amount sufficient to facilitate the entry of the environmental fluid without causing the disruption of the impermeable coating). The swelling agents can include, but are not limited to hydrophilic pharmaceutically acceptable compounds with various swelling rates in water. The swelling agent can include, but is not limited to at least one pharmaceutically acceptable hydrophilic compound, having a swelling rate or swelling amount in water at about 25° C. that is: in certain embodiments greater than or equal to at least about 10% by weight (wt/wt); in other embodiments greater than or equal to at least about 15% by weight (wt/wt); or in still other embodiments greater than or equal to at least about 20% by weight (wt/wt). Non-limiting examples of swelling agents that can be used in the modified release overcoat of certain embodiments include crosslinked polyvinylpyrrolidones (e.g. polyplasdone, crospovidone and mixtures thereof), crosslinked carboxyalkylcelluloses, crosslinked carboxymethylcellulose (e.g. crosslinked sodium croscarmellose), hydrophilic polymers of high molar mass (i.e., which can be, but are not limited to being greater than or equal to about 100,000 Dalton) which can include, but are not limited to: polyvinylpyrrolidone(s), polyalkylene oxides (e.g. polyethylene oxide, polypropylene oxide, and mixtures thereof), hydroxyalkylcelluloses (e.g. hydroxypropylcellulose, hydroxypropylmethylcellulose and mixtures thereof), carboxyalkylcellulose (e.g. carboxymethylcellulose), modified starch (e.g. sodium glycolate), starch or natural starch (e.g. corn, wheat, rice, potato and mixtures thereof), cellulose (i.e. which can be, but is not limited to being in powder form or microcrystalline form), sodium alginate, potassium polacriline, and corresponding blends or mixtures thereof. In certain embodiments, non-limiting examples of the swelling agent can also include the following sub-set of compounds: crosslinked polyvinylpyrrolidone (e.g. polyplasdone, crospovidone or mixtures thereof), crosslinked carboxyalkylcelluloses (e.g. crosslinked carboxymethylcelluloses such as crosslinked sodium croscarmellose), and mixtures thereof. In other embodiments, the swelling agent can be a nitrogen containing polymer, non-limiting examples of which can include polyvinylpyrrolidone, crosslinked polyvinylpyrrolidone and mixtures thereof. The concentration of the swelling agent in the modified release overcoat of certain embodiments that comprise a microparticle core can be from about 3% to about 40% by weight of the microparticle. For example, in certain embodiments the concentration of the swelling agent in the modified release overcoat is from about 4% to about 30%, and in other embodiments from about 5% to about 25% by weight of the microparticle.

Glidant(s) can be included in the modified release overcoat to improve the flowability of powder and/or granules during manufacturing. Non-limiting examples of glidants that can be used in the modified release overcoat include magnesium stearate, calcium silicate, magnesium silicate, talc, glyceryl monostearate, and mixtures thereof. In at least one embodiment the glidants include magnesium stearate and talc. The glidant(s) can each be present in an amount of from about 0.1% to about 80%, for example about 10%, about 20%, about 30%, about 40%, about 50%, about 60% or about 70% of the modified release overcoat dry weight. For example in certain embodiments the glidants are each present in an amount of from about 0.5% to about 20%, in other embodiments from about 0.8% to about 10%, and still other embodiments at about 1.5% of the modified release overcoat weight. In at least one embodiment the glidants are present in an amount of about 5.8% of the modified release overcoat dry weight.

Antiadherent(s) (also known as antitacking agents) can be included in the modified release overcoat to reduce the adhesion between the powder or granules and the punch faces and thus prevent tablet sticking to the punches during manufacturing. Non-limiting examples of antiadherents include adipic acid, magnesium stearate, calcium stearate, zinc stearate, hydrogenated vegetable oils, sterotex, glyceryl monostearate, talc, sodium benzoate, sodium lauryl sulfate, magnesium lauryl sulfate, and mixtures thereof. In at least one embodiment the antiadherent is talc. Talc can also function as a wetting agent, glidant and/or lubricant.

Lubricant(s) can be included in the modified release overcoat to reduce friction during manufacturing. For example, a lubricant can reduce friction between the solid and the die wall during tablet formation and ejection. Lubricants can help prevent ingredients from clumping together and from sticking to the tablet punches or capsule filling machine. Non-limiting examples of lubricants that can be used in the modified release overcoat include adipic acid, magnesium stearate, calcium stearate, zinc stearate, calcium silicate, magnesium silicate, hydrogenated vegetable oils, sodium chloride, sterotex, polyoxyethylene, glyceryl monostearate, talc, polyethylene glycol, sodium benzoate, sodium lauryl sulfate, magnesium lauryl sulfate, sodium stearyl fumarate, light mineral oil, waxy fatty acid esters such as glyceryl behenate, (i.e. COMPRITOL™), STEAR-O-WET™ and MYVATEX™ TL. Combinations of these lubricants are operable. In at least one embodiment the lubricant is selected from magnesium stearate, talc and mixtures thereof.

Emulsifying agent(s) (also called emulsifiers, emulgents or surfactants) can be included in the modified release overcoat to reduce or overcome surface tension effects, facilitate actual emulsification during manufacture of the overcoat, and/or to ensure emulsion stability during the shelf-life of the product. The emulsifying agent(s) that can be used in the modified release overcoat of such embodiments can be anionic, cationic, nonionic, or amphoteric. Examples of emulsifying agents include naturally occurring materials and their semi synthetic derivatives, such as the polysaccharides, as well as glycerol esters, cellulose ethers, sorbitan esters (e.g. sorbitan monooleate or SPAN™ 80), and polysorbates (e.g. TWEEN™ 80). Further non-limiting examples of emulsifying agents include sodium lauryl sulfate, sodium dodecyl sulfate, sorbitan esters, polysorbates, pluronics, potassium laurate, and mixtures thereof. Combinations of emulsifying agents are operable. In at least one embodiment the emulsifying agent is TWEEN™ 80, polyoxyethylene sorbitan monooleate, or a mixture thereof. The emulsifying agent(s) can be present in an amount of from about 0.01% to about 5%, for example about 1%, about 2%, about 3% or about 4% by weight of the modified release overcoat dry weight. For example in certain embodiments the emulsifying agent is present in an amount of from about 0.05% to about 1.5%, in other embodiments from about 0.05% to about 0.5%, and in still other embodiments at about 0.11% by weight of the modified release overcoat dry weight.

Anti-foaming agent(s) can be included in the modified release overcoat to reduce frothing or foaming during manufacture of the modified release overcoat. Non-limiting examples of anti-foaming agents include simethicone, polyglycol and silicon oil. Combinations of anti-foaming agents are operable. In at least one embodiment the anti-foaming agent is Simethicone C. The anti-foaming agent can be present in an amount of from about 0.01% to about 10%, for example about 1%, about 2%, about 3%, about 4%, about 5%, about 6%, about 7%, about 8% or about 9% of the modified release overcoat dry weight. For example in certain embodiments the anti-foaming agent is present in an amount of from about 0.05% to about 5%, in other embodiments from about 0.1% to about 1%, and in still other embodiments at about 0.58% by weight of the modified release overcoat dry weight.

Plasticizer(s) can be included in the modified release overcoat to modify the properties and characteristics of the polymers used in the overcoat for convenient processing during manufacturing (e.g. provide increased flexibility and durability during manufacturing). As used herein, the term “plasticizer” includes any compound(s) capable of plasticizing or softening a polymer or binder used in the present invention. Certain plasticizers can increase the elasticity and/or pliability of a coat, thereby decreasing the coat's brittleness. Once the modified release overcoat has been manufactured, certain plasticizers can function to increase the hydrophilicity of the overcoat in the environment of use. During manufacture of the modified release overcoat, the plasticizer can lower the melting temperature or glass transition temperature (i.e. softening point temperature) of the polymer or binder. The addition of a plasticizer, such as a low molecular weight PEG, generally broadens the average molecular weight of a polymer in which they are included thereby lowering its glass transition temperature or softening point. Plasticizers can also generally reduce the viscosity of a polymer. Non-limiting examples of plasticisers that can be used in the modified release overcoat include acetylated monoglycerides, acetyltributyl citrate, butyl phthalyl butyl glycolate, dibutyl tartrate, diethyl phthalate, dimethyl phthalate, ethyl phthalyl ethyl glycolate, glycerin; propylene glycol, triacetin, tripropioin, diacetin, dibutyl phthalate, acetyl monoglyceride, acetyltriethyl citrate, polyethylene glycols, castor oil, rape seed oil, olive oil, sesame oil, triethyl citrate, polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, polyols (e.g. polyethylene glycol) of various molecular weights, and mixtures thereof. In at least one embodiment the plasticizer used in the modified release overcoat comprises acetyltributyl citrate, triacetin, dibutyl sebacate, and mixtures thereof. The plasticizer can be present in an amount of from about 1% to about 80%, for example about 5%, about 10%, about 20%, about 30%, about 40%, about 50%, about 60% or about 70% of the modified release overcoat dry weight. For example, in certain embodiments the plasticizer is present in an amount of from about 5% to about 50%, in other embodiments from about 10% to about 40%, and in still other embodiments at about 20% of the modified release overcoat dry weight.

In certain embodiments where the modified release overcoat surrounds a microparticle, the modified release overcoat can be present in an amount of from about 1% to about 99%, for example about 10%, about 20%, about 30%, about 40%, about 50%, about 60%, about 70%, about 80%, or about 90% by weight of the microparticle core dry weight, depending at least in part on the choice of polymer; the ratio of polymer:pore former (if a pore former is used); and the total surface area of the microparticle core. Since a certain thickness of modified release overcoating has to be achieved in order to achieve the desired dissolution profile, the amount of modified release polymer coating required during manufacture is related to the total surface area of the batch of microparticle cores that requires a modified release overcoating. The modified released polymer surface area coverage can range from about 0.5 mg/cm² to about 30 mg/cm², for example about 1 mg/cm², about 5 mg/cm², about 10 mg/cm², or about 20 mg/cm². For example in certain embodiments the surface area coverage of the modified release polymer is from about 0.6 mg/cm² to about 20 mg/cm², and in other embodiments from about 1 mg/cm² to about 5 mg/cm². In at least one embodiment, EUDRAGIT® NE30D is used as the modified release polymer at a surface area coverage of about 10 mg/cm². One approach to estimate the total surface area of a multiparticulate batch is the permeability method according to Blaine (ASTM Des. C 205-55), which is based upon the mathematical model of laminar flow through capillaries arranged in parallel. In the absence of an accurate determination of total surface area of a microparticle, the amount of modified release polymer to be applied can be expressed as a percentage of the uncoated microparticle.

In certain embodiments where the modified release overcoat surrounds a microparticle, the modified release polymer can be present in an amount of from about 1% to about 99% by weight, for example about 5% by weight, about 10% by weight, about 20% by weight, about 30% by weight, about 40% by weight, about 50% by weight, about 60% by weight, about 70% by weight, about 80% by weight, or about 90% by weight of the modified release overcoated microparticle, depending at least in part on the modified release profile desired. For example, in certain embodiments the modified release polymer is present in an amount of from about 5% to about 80%, and in other embodiments from about 10% to about 50% by weight of the overcoated microparticle. In at least one embodiment wherein the modified release polymer is EUDRAGIT® NE30D, EUDRAGIT® NE40D (Rohm America LLC), KOLLICOAT® SR 30D, or a mixture thereof, the polymer is present in an amount of from about 1% to about 50%; in other embodiments from about 5% to about 30%; and in still other embodiments at about 15% by weight of the modified release overcoated microparticle. In at least one embodiment wherein the modified release polymer is ethylcellulose, the polymer is present in an amount of from about 1% to about 99% by weight of the modified release overcoated microparticle; in other embodiments from about 5% to about 50%; and in still other embodiments at about 20% by weight of the overcoated microparticle. In at least one embodiment wherein the modified release polymer is ETHOCEL™, an ethyl cellulose grade PR100, PR45, PR20, PR10, PR7 polymer, or a mixture thereof, the polymer is present in an amount of from about 5% to about 30% by weight of the overcoated microparticle; in other embodiments from about 10% to about 25%; and in still other embodiments at about 20% by weight of the modified release overcoated microparticle.

In certain embodiments, the diameter of the microparticles (with or without the modified release overcoat) is from about 50 μm to about 800 μm for example about 100 μm, about 200 μm, about 300 μm, about 400 μm, about 500 μm, about 600 μm, or about 700 μm. For example, in certain embodiments the diameter of each of the microparticles range from about 100 μm to about 600 μm, and in other embodiments from about 150 μm to about 450 μm.

Particle size evaluation of microparticles with a diameter range of approx 50 micron to approx 800 micron can be assessed in accordance with USP method 786 using US mesh numbers 20 and 270.

It is contemplated that in alternative embodiments, other excipients consistent with the objects of the present invention can also be used in the modified release overcoat.

In at least one embodiment, the modified release overcoat includes about 96% EUDRAGIT® NE30D, about 1.9% Magnesium stearate, about 1.9% Talc, about 0.04% TWEEN® 80, and about 0.19% Simethicone C, when expressed as percentage by weight of the modified release overcoat composition dry weight. In at least one other embodiment, the modified release overcoat includes about 87.62% EUDRAGIT® NE30D, about 5.84% Magnesium stearate, about 5.84% Talc, about 0.12% TWEEN® 80, and about 0.58% Simethicone C by weight of the modified release overcoat dry weight. In at least one further embodiment, the microparticle modified release overcoat includes about 68% ethylcellulose, about 17% glyceryl monostearate and about 15% acetyl tributylcitrate when expressed as percentage by weight of the dry modified release overcoat composition dry weight.

In certain embodiments, the modified release polymer is present in an amount of from about 20% to about 99%, preferably from about 50% to about 95%, and more preferably from about 60% to about 90% of the modified release overcoat formulation. In such embodiments, an anti-foaming agent is present in an amount of from about 0.01% to about 10%, preferably from about 0.05% to about 1%, and more preferably from about 0.1% to about 0.3% of the modified release overcoat formulation. In such embodiments, an emulsifying agent is present in an amount of from about 0.01% to about 0.25%, preferably from about 0.01% to about 0.15%, and more preferably from about 0.01% to about 0.07% of the modified release overcoat formulation. In such embodiments, one or more lubricants is present, each in an amount of from about 0.1% to about 80%, preferably from about 0.5% to about 20% and more preferably from about 0.8% to about 10% of the modified release overcoat formulation. In such embodiments, water is present in an amount of from about 2% to about 70%, preferably from about 10% to about 40%, and more preferably from about 15% to about 30% of the modified release overcoat formulation. In at least one embodiment, the modified release overcoat suspension formulation includes about 75% modified release polymer dispersion; about 0.15% anti-foaming agent; about 0.03% emulsifying agent; about 3% lubricant; and about 21.82% water. In at least one embodiment the modified release polymer is a polyacrylate dispersion (e.g. EUDRAGIT® NE30D), the anti-foaming agent is Simethicone C, the emulsifying agent is TWEEN® 80, and the lubricant includes Magnesium Stearate and Talc.

In certain embodiments the weight gain from the modified release overcoat over the microparticle core is from about 5% to about 90%, preferably from about 10% to about 55%, and more preferably from about 15% to about 45%. In at least one embodiment the weight gain from the modified release overcoat over the microparticle core is about 35%.

In certain embodiments the modified release overcoat is a semi-permeable coat comprising modified release polymers that include a water-insoluble, water-permeable film-forming polymer, and a water-soluble polymer. Non-limiting examples of water-insoluble, water-permeable film-forming polymers useful for the modified release overcoat of these embodiments include cellulose ethers, cellulose esters, and polyvinyl alcohol. For example, these water-insoluble, water-permeable film forming polymers can be the ethyl celluloses, and can be selected from the following: ethyl cellulose grades PR100, PR45, PR20, PR10 and PR7 (ETHOCEL®, Dow), and any combination thereof. In at least one embodiment ethyl cellulose grade PR 100 is the water-insoluble, water-permeable film-forming polymer used in the modified release overcoat. In certain embodiments the amount of the water-insoluble water-permeable film-forming polymer can vary from about 1% to about 12% by weight of the dosage form (e.g. tablet) dry weight. For example, in certain embodiments the amount of the water-insoluble water-permeable film-forming polymer is present in an amount from about 5% to about 10%, and in other embodiments from about 6% to about 8% by weight of the dosage form dry weight. With respect to the modified release overcoat itself, the amount of water-insoluble water-permeable film-forming polymer in certain embodiments can be from about 35% to about 60% by weight of the modified release overcoat dry weight, and in other embodiments from about 40% to about 50% by weight of the modified release overcoat dry weight. In certain other embodiments the amount of water-insoluble water-permeable film-forming polymer is from about 2% to about 5% by weight of the dosage form dry weight, and in other embodiments from about 3% to about 4% by weight of the dosage form dry weight. With respect to the modified release overcoat itself, the water-insoluble water-permeable film-forming polymer in certain embodiments can be present in an amount of about 40% by weight of the modified release overcoat dry weight. Non-limiting examples of water-soluble polymers useful for certain embodiments of the modified release overcoat include polyvinylpyrrolidone, hydroxypropyl methylcellulose, hydroxypropyl cellulose and mixtures thereof. In at least one embodiment the water-soluble polymer is polyvinylpyrrolidone (POVIDONE® USP). The amount of water-soluble polymer can vary from about 1.5% to about 10% by weight of the dosage form dry weight. For example, in certain embodiments the amount of water-soluble polymer is from about 3% to about 8%, and in other embodiments at about 4% by weight of the dosage form dry weight. With respect to the modified release overcoat itself, in certain embodiments the amount of water-soluble polymer present is from about 25% to about 55% by weight, for example about 30% by weight, about 35% by weight, about 40% by weight, about 45% by weight, or about 50% by weight of the modified release overcoat dry weight. For certain embodiments the amount of water-soluble polymer can be from about 3% to about 5% by weight of the dosage form dry weight, and from about 25% to about 50% by weight of the modified release overcoat dry weight. For certain other embodiments the amount of water-soluble polymer present can be from about 2% to about 5% of the dosage form dry weight and about 40% to about 50% by weight of the modified release overcoat dry weight.

In certain embodiments where the modified release overcoat comprises a water-insoluble, water-permeable film-forming polymer and a water-soluble polymer, the modified release overcoat can further comprise a plasticizer. The use of plasticizers in the modified release overcoat is optional. In at least one embodiment the modified release overcoat does not include a plasticizer. Non-limiting examples of the plastizer that can be used in the modified release overcoat include polyethylene glycol 4000, dibutyl sebacate and mixtures thereof. The amount of plasticizer for the modified release overcoat of certain embodiments can vary in an amount of from about 0.5% to about 4% by weight of the dosage form (e.g. tablet) dry weight. For example, in certain embodiments the plasticizer can be present in an amount of from about 2% to about 3% by weight of the dosage form dry weight. For certain embodiments the amount of plasticizer present in the modified release overcoat can be from about 1% to about 4% by weight of the dosage form dry weight. For certain other embodiments the amount of plasticizer present can be from about 0.5% to about 4% by weight of the dosage form dry weight. In certain further embodiments the plasticizer can be present in an amount of from about 6% to about 30% by weight of the modified release overcoat dry weight. For example, in certain embodiments the plasticizer is present in an amount of about 12% by weight of the modified release overcoat dry weight. The ratio of water-insoluble water-permeable film forming polymer:plasticizer:water-soluble polymer for the modified release overcoat of certain embodiments of the invention described herein can vary from about 3:1:4 to about 5:1:2. For example, in certain embodiments the ratio of water-insoluble water-permeable film forming polymer:plasticizer:water-soluble polymer for the modified release overcoat is about 4:1:3. For certain other embodiments the ratio of the water-insoluble water-impermeable film-forming polymer:plasticizer:water-soluble polymer in the modified release overcoat is from about 7:2:6 to about 19:5:18. In at least one embodiment the ratio of water-insoluble water-permeable film forming polymer:plasticizer:water-soluble polymer for the modified release overcoat is about 13:4:12.

In certain embodiments the modified release overcoat can be a stable monolithic coating comprising an aqueous dispersion of a neutral ester copolymer without any functional groups, a poly glycol having a melting point greater than about 55° C., and one or more pharmaceutically acceptable excipients. The coat composition is coated onto the dosage form and cured at a temperature at least equal to or greater than the melting point of the poly glycol. The coating formulation of these embodiments is quite versatile in that it can be used to coat a variety of drug cores and can be easily manipulated to obtain the desired drug release profile. Non-limiting examples of neutral ester copolymers without any functional groups include EUDRAGIT® NE30D, EUDRAGIT® NE40D (Röhm America LLC), and mixtures thereof. In at least one embodiment the polymer is EUDRAGIT® NE30D, which can be present in an amount of from about 1% to about 35% by weight of the modified release overcoat, depending on the release profile desired. In at least one embodiment the modified release overcoat comprises an aqueous dispersion of an ethylcellulose. Non-limiting examples of aqueous dispersions of an ethylcellulose include SURELEASE® (Colorcon, Inc., West Point, Pa., U.S.A.), and AQUACOAT® (FMC Corp., Philadelphia, Pa., U.S.A.). In certain embodiments hydrophilic agents can also be included in the modified release overcoat to promote wetting of the coat when in contact with gastrointestinal fluids. Non-limiting examples of such hydrophilic agents include hydrophilic water-soluble polymers such as hydroxypropyl methylcellulose (HPMC), hydroxypropyl cellulose (HPC) and combinations thereof. In at least one embodiment, HPMC is the hydrophilic water-soluble polymer. If hydrophilic agents are to be included in the coat composition, the agents can be present in an amount from about 0.1% to about 10% by weight of the coating composition. For example, in certain embodiments the hydrophilic agents are present in an amount of from about 0.1% to about 5%, and in other embodiments from about 0.1% to about 3% by weight of the modified release overcoat composition. Non-limiting examples of the poly glycol with a melting point of greater than about 55° C. include polyethylene glycol 6000, polyethylene glycol 8000, polyethylene glycol 10000, polyethylene glycol 20000, and mixtures thereof. In at least one embodiment, the poly glycol is polyethylene glycol 8000. The poly glycol can be present in an amount of from about 0.1% to about 5% by weight of the coat. Other examples of suitable polyglycol derivatives having a melting point of at least about 55° C. include, but are not limited to, Poloxamer 188, Poloxamer 338, Poloxamer 407, Polyethylene Oxides, Polyoxyethylene Alkyl Ethers, Polyoxyethylene Stearates, and mixtures thereof. In addition to the copolymers and the poly glycol, certain embodiments of the modified release overcoat formulation can comprise at least one pharmaceutically acceptable excipient, such as an antiadherent, anti-foaming agent, and/or emulsifying agent. In certain embodiments antiadherents are also included in the modified release overcoat formulation. In at least one embodiment the antiadherent is talc. The amount of antiadherent in the modified release overcoat composition of certain embodiments can be in the range from about 1% to about 15% by weight of the modified release overcoat dispersion. For example, in certain embodiments the antiadherent is present in an amount of from about 1% to about 7% by weight of the modified release overcoat dispersion. In certain embodiments anti-foaming agents are also included in the modified release overcoat formulation. In at least one embodiment the anti-foaming agent is simethicone. The anti-foaming agent can be present in the modified release overcoat of certain embodiments in an amount from about 0.1% to about 0.4% by weight of the modified release overcoat composition. In certain embodiments emulsifying agents can be included in the modified release overcoat. In at least one embodiment the emulsifying agent is Polysorbate 80 (polyoxyethylene sorbitan mono-oleate) (TWEEN™ 80). The emulsifying agent can be present in the modified release overcoat of certain embodiments in an amount from about 0.1% to about 0.3% by weight of the modified release overcoat composition. The modified release overcoat of such embodiments can be quite versatile. For example, the length and time for a delay can be controlled by rate of hydration and the thickness of the coat. The drug release rate subsequent to the delay can be determined by the thickness and permeability of the hydrated coat. Thus, it is possible to regulate the rate of hydration and permeability of the modified release overcoat so that the desired controlled-release drug profile can be achieved. The coat thickness will depend on the controlled release profile desired. Other parameters in combination with the thickness of the coat include varying the concentrations of some of the ingredients of the overcoat composition described and/or varying the curing temperature and length of curing time.

The manufacturing process for the modified release overcoat can be as follows: Water is split into two portions of about 15% and about 85%. The anti-foaming agent and the emulsifying agent are then added to the about 15% water portion, and mixed at about 300 rpm to form portion A. In at least one embodiment, the anti-foaming agent is Simethicone C, and the emulsifying agent is TWEEN™ 80. A first lubricant is then added to the about 85% water portion and mixed at about 9500 rpm to form portion B. In at least one embodiment, the first lubricant is talc. Then portion A is mixed with portion B, a second lubricant is slowly added, and mixed at about 700 rpm overnight. In at least one embodiment, the second lubricant is magnesium stearate. Finally, an aqueous dispersion of a neutral ester copolymer is added and mixed for about 30 minutes at about 500 rpm. In at least one embodiment, the aqueous dispersion of a neutral ester copolymer is EUDRAGIT® NE30D. The resultant modified release overcoat solution can then be used to overcoat the osmotic subcoated microparticles to about a 35% weight gain (over the uncoated microparticle core) with the following parameters: An inlet temperature of from about 10° C. to about 60° C., preferably from about 20° C. to about 40° C., and more preferably from about 25° C. to about 35° C.; an outlet temperature of from about 10° C. to about 60° C., preferably from about 20° C. to about 40° C., and more preferably from about 25° C. to about 35° C.; a product temperature of from about 10° C. to about 60° C., preferably from about 15° C. to about 35° C., and more preferably from about 22° C. to about 27° C.; an air flow of from about 10 c.m/h to about 180 c.m/h, preferably from about 40 c.m/h to about 120 c.m/h, and more preferably from about 60 c.m/h to about 80 c.m/h; and an atomizing pressure of from about 0.5 bar to about 4.5 bar, preferably from about 1 bar to about 3 bar, and more preferably about 2 bar. The resultant overcoated microparticles can then be discharged from the coating chamber and ovencured with the following parameters: A curing temperature of from about 20° C. to about 65° C., preferably from about 30° C. to about 55° C., and more preferably about 40° C.; and a curing time of from about 2 hours to about 120 hours, preferably from about 10 hours to about 40 hours, and more preferably about 24 hours. Any other technology resulting in the coating formulation of the modified release overcoat consistent with the objects of the invention can also be used.

The manufacturing process for the modified release overcoat for certain other embodiments can be as follows: One or more water-insoluble polymer(s) (and possibly one or more other excipients) are applied onto the osmotic subcoated microparticles by a dry powder coating technique. A mixture of EUDRAGIT® RS and Talc powder (1:1 w/w) and an emulsion of triethyl citrate (about 45% w/w of total emulsion) as a plasticizer in an aqueous about 10% w/w HPMC binder solution (about 55% w/w of total emulsion) are fed/sprayed through separate inlets onto the subcoated microparticles in a fluidised bed (GLATT® GPCG-1, Wurster insert), according to the following parameters: inlet air temperature of from about 40° C. to about 45° C.; product temperature of from about 34° C. to about 36° C.; outlet air temperature of from about 32° C. to about 36° C.; air flow rate of from about 60c.m/h to about 80 c.m/h; atomizing air pressure of from about 1 bar to about 1.5 bar; powder feed rate of from about 10 g/min to about 12 g/min; and spray rate of from about 3 g/min to about 5 g/min.

In certain embodiments where the modified release overcoat comprises a water-insoluble, water-permeable film-forming polymer, a water-soluble polymer, and an optional plasticizer, the preparation and application of the modified release overcoat can be as follows: The water-insoluble water-permeable film-forming polymer (e.g. ethylcellulose), and the plasticizer (e.g. polyethylene glycol 4000), are dissolved in an organic solvent (e.g. a mixture of ethyl alcohol). In the manufacture of embodiments that do not require a plasticizer, the water-insoluble water-permeable film-forming polymer can be dissolved in the organic solvent without the plasticizer. The water-soluble polymer (e.g. polyvinyl pyrrolidone) is next added until a homogenous mixture is achieved. The resulting modified release overcoat solution is then sprayed onto the osmotic subcoated cores using a coater, fluidized bed apparatus or any other suitable coating apparatus known in the art until the desired weight gain is achieved. The cores coated with the modified release overcoat are subsequently dried. In the manufacture of embodiments that have a moisture barrier, the modified release overcoat is dried before the moisture barrier is applied.

A further example of the coating process for certain embodiments where the modified release overcoat comprises a water-insoluble, water-permeable film-forming polymer, a water-soluble polymer, and an optional plasticizer, is as follows: The modified release overcoat solution is prepared by dissolving the water insoluble polymer (e.g. ethylcellulose) and water soluble polymer (e.g. polyvinylpyrrolidone) and an ethyl alcohol mixture while mixing and is followed with the addition of the plasticizer(s) (e.g. polyethylene glycol 4000 and dibutyl sebacate). Once completely dissolved, the solution is homogenized to obtain a uniform mixture of appropriate viscosity. This procedure assures a complex mix of a water permeable film to control the release of the drug. The composition of the solution can be formulated to contain various levels of the water insoluble polymer and water soluble polymer and a mix of the plasticizer(s). The release function is further controlled by the film thickness applied and measured as weight gain of solids in the coating required. Dosage forms (e.g. tablets) are coated in a perforated coating pan with control of pan speed (e.g. from about 8 rpm to about 14 rpm, and in some cases about 12 rpm), spray rate (e.g. from about 150 gm/min to about 250 gm/min, and in some cases about 200 gm/min), atomization pressure (e.g. from about 15 psi to about 25 psi, and in some cases about 20 psi), supply volume (e.g. from about 800 to about 1000 cubic ft/min, and in some cases about 900 cubic ft/min), and air temperature (e.g. from about 50° C. to about 60° C., and in some cases about 55° C.), monitored through a bed temperature and/or outlet temperature of from about 38° C. to about 42° C., and in some cases about 40® C. On completion of the coating cycle, dosage forms are dried and unloaded into bulk containers. The printing process comprises the transfer of a print image from a print plate covered with edible black ink and transferred via a print roll or print pad onto the surface of the dosage forms (e.g. tablets). The printed dosage forms are transferred through a drying element prior to discharging into bulk containers. Samples for final testing are taken throughout the printing process. The skilled artisan will appreciate that controlling the permeability can control the release of the drug and/or the amount of coating applied to the cores. The permeability of the modified release overcoat, can be altered by varying the ratio of the water-insoluble, water-permeable film-forming polymer:plasticizer:water-soluble polymer and/or the quantity of coating applied to the core. A more extended release can be obtained with a higher amount of water-insoluble, water-permeable film forming polymer. The addition of other excipients can also alter the permeability of the modified release overcoat. For example, if it is desired that the dosage form further comprise an expanding agent, the amount of plasticizer in the modified release overcoat could be increased to make the coat more pliable, as the pressure exerted on a less pliable coat by the expanding agent could rupture the coat. Further, the proportion of the water-insoluble water-permeable film forming polymer and water-soluble polymer can also be altered depending on whether a faster or slower dissolution and/or release profile is desired. Depending on the dissolution or in-vivo release profile desired, the weight gained after coating the core with the modified release overcoat can vary from about 3% to about 30% of the weight of the dry dosage form. For example in certain embodiments, the weight gain is from about 10% to about 17% of the weight of the dry dosage form. In other embodiments the weight gain can vary from about 7% to about 10% of the weight of the dry dosage form.

In certain embodiments where the modified release overcoat comprises an aqueous dispersion of a neutral ester copolymer without any functional groups and a poly glycol having a melting point greater than about 55° C., the preparation and application of the modified release overcoat can for example be as follows: the modified release overcoat can be applied onto a core comprising an effective amount of the drug by a process which involves the atomization (spraying) of the coating solution or suspension onto a bed of the tablet cores. Some examples of equipment suitable for film coating include: Accela Cota (Manesty Machines, Liverpool, UK), Hi-Coater (Freund Company, Japan), Driacoater (Driam Metallprodukt GmbH, Germany), HTF/150 (GS, Italy), and IDA (Dumoulin, France). Examples of units that function on a fluidized-bed principle include: Aeromatic (Fielder, Switzerland and UK) and Glatt AG (Switzerland). In at least one embodiment, the apparatus used for film coating is the Accela Cota. The coating fluid can be delivered to the coating apparatus from a peristaltic pump at the desired rate and sprayed onto the rotating or fluidizing tablet cores. The tablet cores are pre-warned to about 30° C. During the coating process, the product temperature range is maintained between about 25° C. and about 35° C. by adjusting the flow rate of the inlet and outlet air, temperature of the inlet air and spray rate. A single layer of coat is applied and once spraying is complete, the coated tablet cores are dried between about 30° C. to about 40° C. for from about 3 to about 5 minutes at a low pan speed and low air flow. The pan is readjusted to jog speed, and drying continues for from about 12 to about 15 minutes. The coated tablet cores are placed onto a tray and cured (post coating thermal treatment) in an electrical or steam oven at a temperature above the temperature of the melting point of the polyethylene glycol or derivative thereof. The curing temperature is preferably greater than the melting point of the polyethylene glycol or derivative thereof. The curing time is preferably from about 2 to about 7 hours. The cured coated tablets are subsequently cooled to room temperature.

In certain embodiments, a moisture barrier coat is applied directly onto the modified release overcoat, and in other embodiments directly onto the additional overcoat. In other embodiments a moisture barrier coat is not included in the dosage form.

The moisture barrier coat of certain embodiments can impede or retard the absorption of moisture into the composition. The proportion of the components of the moisture barrier and the amount of the moisture barrier applied onto the composition is such that the moisture barrier does not fall within the USP definition and requirement for an enteric coat.

The moisture barrier coat can include an enteric polymer (e.g. acrylic polymer), a permeation enhancer, and an optional plasticizer. In certain embodiments, the enteric polymer is an acrylic polymer. For example, the acrylic polymer can be a methacrylic acid copolymer type C [poly(methacrylic acid, methyl methacrylate) 1:1] available commercially under the trade name EUDRAGIT® (e.g. EUDRAGIT® L 30 D-55). In certain embodiments the methacrylic acid copolymer can be present in an amount from about 1% to about 3% of the dosage form (e.g. tablet) dry weight. In at least one embodiment the amount of the methacrylic acid copolymer is present at about 2.5% of the dosage form dry weight. With respect to the moisture barrier coat itself, the amount of the methacrylic acid copolymer in certain embodiments can be present in an amount of from about 55% to about 70% by weight of the moisture barrier coat dry weight. In at least one embodiment the methacrylic acid copolymer is present in an amount of about 60% of the moisture barrier coat dry weight.

In certain embodiments the moisture barrier coat also comprises a plasticizer. Non-limiting examples of plasticizers useful for the moisture barrier coat described herein include acetylated monoglycerides; acetyltributyl citrate, butyl phthalyl butyl glycolate; dibutyl tartrate; diethyl phthalate; dimethyl phthalate; ethyl phthalyl ethyl glycolate; glycerin; propylene glycol; triacetin; tripropioin; diacetin; dibutyl phthalate; acetyl monoglyceride; acetyltriethyl citrate, polyethylene glycols; castor oil; rape seed oil, olive oil, sesame oil, triethyl citrate; polyhydric alcohols, glycerol, glycerin sorbitol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylphthalate, dibutylsebacate, glyceroltributyrate, and mixtures thereof, polyols (e.g. polyethylene glycol) of various molecular weights, and mixtures thereof. In certain embodiments, the plasticizer in the moisture barrier coat comprises a combination of triethyl citrate and polyethylene glycol 4000 (e.g. CARBOWAX® 4000). In certain of these embodiments, the ratio of triethyl citrate to polyethylene glycol 4000 is about 1:2. The plasticizer in the moisture barrier coat of certain embodiments can be present in an amount from about 0.2% to about 0.5% of the dosage form (e.g. tablet) dry weight. With respect to the moisture barrier coat itself, the plasticizer, if present, can be present in an amount of from about 1% to about 30% by weight of the moisture barrier coat dry weight. In at least one embodiment the plasticizer is present in an amount of from about 10% to about 14% of the moisture barrier coat dry weight.

The moisture barrier coat of certain embodiments can further comprise a permeation enhancer that can increase its hydrophilicity, and can also act as a glidant. The permeation enhancer can be a hydrophilic substance that allows water to enter without substantial physical disruption of the coating. Non-limiting examples of the permeation enhancer includes: hydrophilic polymers such as hydroxypropylmethylcellulose, cellulose ethers (e.g. hydroxyalkylcelluloses, and carboxyalkylcelluloses), and protein-derived materials of these polymers. Also, synthetic water-soluble polymers can be used as permeation enhancers in the moisture barrier coat, non-limiting examples of which include polyvinylpyrrolidone, cross-linked polyvinyl-pyrrolidone, polyethylene oxide, water-soluble polydextrose, saccharides and polysaccharides, such as pullulan, dextran, sucrose, glucose, lactose, fructose, mannitol, mannose, galactose, sorbitol and mixtures thereof. In at least one embodiment the permeation enhancer includes hydroxypropyl-methylcellulose. Other non-limiting examples of permeation enhancers include alkali metal salts such as aluminium oxidelithium carbonate, sodium chloride, sodium bromide, potassium chloride, potassium sulfate, potassium phosphate, sodium acetate, sodium citrate, and mixtures thereof. The permeation enhancers can also be polymers which are soluble in the environment of use, such as CARBOWAX®, CARBOPOL®, and mixtures thereof. The permeation enhancers embrace diols, polyols, polyhydric alcohols, polyalkylene glycols, polyglycols, poly(a-w)alkylenediols, and mixtures thereof. Other permeation enhancers which can be useful in the formulations of the present invention include starch, modified starch, and starch derivatives, gums, including but not limited to xanthan gum, alginic acid, other alginates, benitonite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, such as potassium polymethacrylate, carrageenan, kappa-carrageenan, lambda-carrageenan, gum karaya, biosynthetic gum, and mixtures thereof. Other permeation enhancers include materials useful for making microporous lamina in the environment of use, such as polycarbonates comprised of linear polyesters of carbonic acid in which carbonate groups reoccur in the polymer chain, microporous materials such as bisphenol, a microporous poly(vinylchloride), micro-porous polyamides, microporous modacrylic copolymers, microporous styrene-acrylic and its copolymers, porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous hiomopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), silicon dioxide, colloidal silica, microcrystalline cellulose and any combination thereof. In at least one embodiment of the invention the permeation enhancer of the moisture barrier coat is silicon dioxide (e.g. SYLOID® 244FP). The amount of permeation enhancer can vary from about 0.5% to about 2% by weight of the dosage form dry weight, and from about 20% to about 40% by weight of the moisture barrier coat dry weight. In at least one embodiment the ratio of the methacrylic acid copolymer:plasticizer:permeation enhancer in the moisture barrier coat is about 13:2:5.

The preparation and application of the moisture barrier coat can for example be as follows: The plasticizer (e.g. a combination of polyethylene glycol 4000 and triethyl citrate) can be first added to water and the mixture mixed to homogeneity. The methacrylic acid co-polymer (e.g. EUDRAGIT® L 30 D-55), is next sieved and added to the plasticizer mixture and mixed to homogeneity. In a separate container the permeation enhancer (e.g. silicon dioxide) is dissolved in water until a homogeneous mixture is achieved. The plasticizer and methacrylic acid copolymer mixture is then combined with the permeation enhancer solution and mixed to homogeneity. The resulting moisture barrier solution is then sprayed onto the coated cores (i.e. coated with the modified release overcoat and/or the additional overcoat) using a tablet coater, fluidized bed apparatus or any other suitable coating apparatus known in the art until the desired weight gain is achieved. The tablets coated with the moisture barrier coat are subsequently dried prior to packaging.

The moisture barrier coat of certain embodiments is applied to the coated tablet cores (i.e. coated with the modified release overcoat and/or the additional overcoat) such that the weight gain is not more than about 6% of the tablet dry weight. In at least one embodiment the weight gain is not more than about 3.5% of the tablet dry weight. The amount of the moisture barrier applied typically does not significantly render the modified release tablet described herein more resistant to gastric fluid. However, the moisture barrier can have an impact on the drug release characteristics.

The moisture barrier coat of certain embodiments does not function as an enteric coat. Even though the methacrylic acid copolymer, EUDRAGIT® L 30 D-55, can be used in enteric coating formulations, its functionality is formulation dependent and on the quantity of the material applied. As is known by the skilled artisan, an enteric coating is applied where a drug may be destroyed or inactivated by gastric juice or where the drug may irritate the gastric mucosa. To meet the requirements for an enteric coat, the test as described in the USP (method A or B) stipulates that after 2 hours in acidic media (0.1N HCl), no individual values of at least six experiments exceed 10% of the drug dissolved and not less than 75% dissolved at 45 minutes in pH 6.8. The moisture barrier coat of certain embodiments does not meet this requirement for the following reasons even though the drug may not be negatively affected in acidic media nor irritate the gastric mucosa: (1) to obtain enteric integrity with a film containing EUDRAGIT® L 30 D-55, a weight gain of between 6% to 8% based on the dry polymer per dosage unit is recommended. The amount of EUDRAGIT® L 30 D-55 solid applied onto the coated cores is not more than about 6%, and in at least one embodiment, is not more than about 3%, (2) if enteric integrity would be required, the dissolution test for the finished product (i.e., the moisture barrier coated tablet cores) at the 2 hour time point would not stipulate a limit of no more than 20%, and (3) analytical tests performed on these coatings indicate that the coatings do not meet all the test requirements as an enteric coated product as defined by USP test methods.

In certain embodiments, an enteric coat is included in the composition to surround the drug-containing core. In certain embodiments an enteric coat is applied directly onto the modified release overcoat, and in other embodiments directly onto the additional overcoat. In other embodiments an enteric coat is not included in the dosage form. Depending upon the composition and/or thickness, the enteric coat of certain embodiments can control the location in the digestive system where the drug is absorbed. For example, the enteric coat can be used to: (i) protect the drug from the destructive action of the enzymes or low pH environment of the stomach; (ii) prevent nausea or bleeding associated with the irritation of the gastric mucosa by the drug; and/or (iii) deliver the drug in an undiluted form in the intestine. The enteric coat can confine the release of the drug to a predetermined region of the gastrointestinal tract. The enteric coat of certain embodiments has a surface that is substantially stable at acidic pH, but breaks down at higher pH to allow release of the drug in the lower stomach or the small intestines. In certain embodiments the enteric coat will provide for protection of the drug at pH's less than about 5 (such as found in the stomach) but can permit drug release at a pH of about 5 or higher (such as found in the upper intestines).

The enteric coat includes at least one enteric polymer. The enteric polymer is a polymeric substance that when used in an enteric coat formulation, is substantially insoluble or stable under acidic conditions exhibiting a pH of less than about 5 (e.g. the upper portion of the gastrointestinal tract such as the stomach), and that is substantially soluble or decomposable under conditions exhibiting a pH of about 5 or more (e.g. intestinal juices such as within the small intestine); and therefore able to permit release of the drug in the regions of the small intestine and not in the stomach. The pH-solubility behavior of the enteric polymers of the enteric coat are such that substantial dissolution of the enteric coating will not occur until the dosage form has emptied from the stomach.

Non-limiting examples of such enteric polymers include carboxymethylethylcellulose, cellulose acetate phthalate, cellulose acetate succinate, methylcellulose phthalate, hydroxymethylethylcellulose phthalate, hydroxypropylmethylcellulose phthalate, hydroxypropylmethylcellulose acetate succinate, polyvinyl alcohol phthalate, polyvinyl butyrate phthalate, polyvinyl acetate phthalate, a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate, a copolymer of methacrylic acid/methyl methacrylate and mixtures thereof.

In at least one embodiment the enteric polymer is EUDRAGIT® L30D (methacrylic copolymer). EUDRAGIT® L30D is an aqueous acrylic resin dispersion, an anionic copolymer derived from methacrylic acid and ethyl acrylate with a ratio of free carboxyl groups to the ester of about 1:1, and a mean molecular weight of approximately 250,000, is supplied as an aqueous dispersion containing about 30% w/w of dry lacquer substance, and is marketed by Rohm-Pharma Co., Germany.

The amount of enteric polymer in the enteric coating is such that it is substantially dissolved during the dosage form's transit time within the small intestine.

In certain embodiments the enteric coat can also include a plasticizer. Non-limiting examples of plasticizers that can be used in the enteric coat include diethyl phthalate, triethyl citrate (Citroflex-2), triacetin, tributyl sebecate, polyethylene glycol, and mixtures thereof.

In certain embodiments the enteric coat can also include an anti-adherent (anti-agglomerant). Non-limiting examples of anti-adherents that can be used in the enteric coat include hydrophobic materials such as talc, magnesium stearate, fumed silica, and mixtures thereof.

In at least one embodiment the enteric coat comprises EUDRAGIT® L30D (methacrylic copolymer), talc and polyethyleneglycol.

In certain embodiments the enteric coat includes an enteric polymer (e.g. methacrylic acid copolymer) in an amount of from about 5% to about 30%; a plasticizer in an amount of from about 1% to about 6%; and an anti-adherent in an amount of from about 0.1% to about 4% by weight of the enteric coat dry weight.

The preparation and application of the enteric coat can for example be as follows: The drug-containing cores can be coated with an enteric film coating suspension comprising an enteric polymer (e.g. EUDRAGIT® L-30-D) and plasticizer (e.g. diethyl phthalate), using a fluid bed coater, such as a Wurster spray coating system or other suitable coating system, and then dried. During preparation of the film coating suspension, a NaOH solution can be added to the suspension until a pH of about 5.0±0.1 is obtained. To prevent clumping of the enteric coated cores, a hydrophobic anti-adherent (e.g. talc) is then added to the film coated beads and blended.

The present invention contemplates combinations of a first drug with at least a second drug, which may or may not be the same as the first drug. For example, in at least one embodiment a composition is provided which comprises a first component that includes a first drug, and a second component that includes at least one second drug (which may or may not be the same drug as the first drug), wherein the two components are present in an amount effective in the treatment or prevention of at least one disease, or for the treatment or mitigation of at least one symptom of the at least one disease, or slowing the progression of at least one disease, or to cause the disease to go into remission, either partially or fully, and to prolong survival as compared to expected survival if not receiving treatment, in a patient or patient in need thereof.

Certain embodiments of the present invention further provide a method for treating or preventing a disease or treating or preventing a symptom of a disease, comprising administering to a patient, or patient in need thereof, an effective amount of a first component that includes a first drug in combination with an effective amount of a second component that includes at least one second drug, which may or may not be the same as the first drug.

In certain embodiments, combination products can be made by providing an additional overcoat to at least partially or completely surround each modified-release overcoated composition. In certain embodiments, a pulsatile release of at least one drug is achieved from the coated compositions. For example this additional overcoat can be an immediate release overcoat that includes at least one drug and at least one low viscosity hydrophilic polymer. In certain embodiments the drug(s) in the additional overcoat can be the same as one or more of any of the drug(s) used in the core. In other embodiments the drug(s) in the additional overcoat is different from the drug(s) in the core. The low-viscosity polymer provides for the immediate release of the drug(s) from the additional overcoat. Non-limiting examples of low-viscosity polymers that can be used in the additional overcoat include polyvinyl pyrrolidone, hydroxyethyl cellulose, hydroxypropyl cellulose, low molecular weight hydroxypropyl methylcellulose (HPMC), polymethacrylate, ethyl cellulose, and mixtures thereof. In at least one embodiment, the low viscosity polymer is a low molecular weight hydrophilic polymer. A wide variety of low molecular weight and low viscosity hydrophilic polymers can be used in the additional overcoat. In at least one embodiment the hydrophilic polymer used in the additional overcoat is a low molecular weight hydroxypropylmethylcellulose (HPMC) polymer. Non-limiting examples of HPMC polymers that can be used in the additional overcoat include PHARMACOAT® 606, PHARMACOAT® 606G, PHARMACOAT® 603, METHOCEL® E3, METHOCEL® E5, METHOCEL® E6, and mixtures thereof. In at least one embodiment, the low-viscosity polymer used in the additional overcoat is PHARMACOAT® 606G. The low viscosity polymer of the additional overcoat can be present in an amount of from about 1% to about 99% of the additional overcoat dry weight. For example, in certain embodiments the low viscosity polymer is present in an amount of from about 15% to about 60%, in other embodiments from about 20% to about 40%, and in still other embodiments at about 30% of the additional overcoat dry weight.

In certain embodiments the additional overcoat can also include a lubricant such as talc. Talc can be present in an amount of from about 1% to about 80%, for example about 10%, about 20%, about 30%, about 40%, about 50%, about 60% or about 70% of the additional overcoat dry weight. Other lubricants that can be used in the additional overcoat of certain embodiments include any one or more of the lubricants used in the modified release overcoat. For example, in certain embodiments the lubricant is present in an amount of from about 5% to about 50%, and in still other embodiments at about 7.5% of the additional overcoat dry weight.

The drug in the additional overcoat can be present in an amount of from about 1% to about 90%, for example about 5%, about 10%, about 20%, about 30%, about 40%, about 50%, about 60%, about 70% or about 80% of the additional overcoat dry weight. For example, in certain embodiments a drug is present in an amount of from about 20% to about 80%, and in still other embodiments at about 62.5% of the additional overcoat dry weight.

In certain embodiments the additional overcoat is designed to achieve a pulsatile release of at least one drug from the coated composition. In at least one embodiment there is provided an immediate release of at least one drug from the additional overcoat in a first phase of drug release, and then a subsequent modified release of at least one drug from the core in a second phase of drug release. In certain embodiments the drug(s) released from the additional overcoat in the first phase of drug release is different from the drug(s) that is released from the core in the second phase of drug release. In certain other embodiments the drug(s) released from the additional overcoat in the first phase of drug release is the same drug(s) that is released from the core in the second phase of drug release.

In certain embodiments the manufacturing process for the additional overcoat can be as follows: Drug and lubricant are dissolved and/or dispersed in water. In at least one embodiment the lubricant is talc. A low viscosity polymer is then added gradually to the solution. In at least one embodiment the low viscosity polymer is PHARMACOAT® 606G. A magnetic stirrer can be used to aid in the mixing of the low viscosity polymer to the solution/dispersion of drug and lubricant. The resultant additional overcoat solution can then be used to coat the modified release microparticles in a fluidized bed granulator, such as a granulator manufactured by GLATT® (Germany) or AEROMATIC® (Switzerland). In at least one embodiment, the microparticles are coated with the additional overcoat solution in a GLATT®-Powder-Coater-Granulator (GPCG 1.1) to about a 20% weight gain (over the uncoated microparticle core) with the following parameters: An inlet temperature of from about 10° C. to about 70° C., preferably from about 30° C. to about 55° C., and more preferably from about 40° C. to about 45° C.; an outlet temperature of from about 10° C. to about 70° C., preferably from about 20° C. to about 45° C., and more preferably from about 30° C. to about 35° C.; a product temperature of from about 10° C. to about 70° C., preferably from about 20° C. to about 45° C., and more preferably from about 30° C. to about 35° C.; an air flow of from about 10 c.m/h to about 180 c.m/h; preferably from about 40 c.m/h to about 120 c.m/h; and more preferably from about 60 c.m/h to about 80 c.m/h; an atomizing pressure of from about 0.5 bar to about 4.5 bar, preferably from about 1 bar to about 3 bar, and more preferably about 2 bar; a curing temperature of from about 10° C. to about 70° C., preferably from about 20° C. to about 50° C., and more preferably from about 30° C. to about 40° C.; and a curing time of from about 5 minutes to about 720 minutes; preferably from about 10 minutes to about 120 minutes, and more preferably about 30 minutes. Any other technology resulting in the coating formulation of the additional overcoat consistent with the objects of the invention can also be used.

In at least one embodiment, the drug, lubricant and low viscosity polymer are coated onto the modified release microparticle cores by a dry powder coating technique. For example, a mixture of drug and talc powder about 89.3% and about 10.7% w/w) and an aqueous 10% w/w HPMC 6 cps binder solution are fed/sprayed through separate inlets onto modified release microparticles in a fluidised bed coater (GLATT® GPCG-1, Wurster insert), with the following parameters: inlet air temperature of from about 40° C. to about 45° C.; product temperature of from about 34° C. to about 36° C.; outlet air temperature of from about 32° C. to about 36° C.; air flow rate of from about 60c.m/h to about 80 c.m/h; atomizing air pressure of from about 1 bar to about 1.5 bar; powder feed rate of from about 10 g/min to about 12 g/min; and spray rate of from about 3 g/min to about 5 g/min.

In addition, certain other embodiments of the present invention allow for combinations of different groups of microparticles into a single dosage form, wherein each group of microparticles has a different release profile and/or functional coating. For example, by combining a first group of immediate release taste-masked or enteric coated microparticles with a second group of delayed release microparticles (e.g. having a modified release overcoat with a delayed release profile), a pulsatile drug release profile or chronotherapeutic profile can be achieved.

In certain embodiments, a dosage form is provided that includes one or more microparticle cores comprising a drug and a spheronization aid; an osmotic subcoat comprising an osmotic agent and an osmotic deposition vehicle that surrounds each microparticle core; a modified release overcoat that surrounds each osmotically subcoated microparticle; and at least one pharmaceutical excipient such as a binder (e.g. polyvinyl alcohol), a lubricant (e.g. glyceryl behenate), and/or filler, that is combined with the coated microparticles and compressed into a tablet. In at least one embodiment the dissolution profile of the coated multiparticles and the effect of the osmotic agent, are not affected by the compression of the microparticles into a tablet. Tablets can be manufactured by compressing the coated microparticles with suitable inert excipients using known compression techniques. The inert excipients can be added to facilitate the preparation and/or improve patient acceptability of the final modified release dosage form as described herein. The additional inert excipients are well known to the skilled artisan and can be found in the relevant literature, for example in the Handbook of Pharmaceutical Excipients.

In certain other embodiments the coated microparticles are filled into capsules.

The forms of administration suitable for the dosage forms of the present invention include oral administration. Non-limiting examples of such dosage forms include tablets and capsules. Other possible dosage forms include pellets, beads or microtablets, which can then be packaged into capsules or compressed into a unitary solid dosage form. Other solid oral dosage forms can be prepared by the skilled artisan, even if such other solid oral dosage forms may be more difficult to commercially manufacture.

The present invention also contemplates combinations of differently coated microparticles into a dosage form to provide a variety of different release profiles. For example, in certain embodiments, microparticles with a delayed release profile can be combined with other microparticles having a sustained release profile to provide a multiple component modified release formulation. In addition, other embodiments can include one or more further components having an immediate release profile. The immediate release component can take the form of uncoated microparticles or powders; microparticles coated with a highly soluble immediate release coating, such as an OPADRY® type coating, as are known to those skilled in the art, or a combination of any of the foregoing. The multiple components can then be blended together in the desired ratio and placed in a capsule, or formed into a tablet. Examples of multiple component modified release formulations can be found in U.S. Pat. No. 6,905,708.

The present invention also contemplates an oral delivery system for delivering drug-containing microparticles in admixture with a fluid. For example, an oral delivery system is provided which comprises a hollow drug formulation chamber. In at least one embodiment, the chamber has a first end and a second end, and contains a formulation in the form of osmotically subcoated microparticles. The system further comprises a fluid passing drug formulation retainer in the first end of the chamber. The retainer prevents release of the microparticles from the first end while permitting fluid entry into the chamber.

The present invention further provides a method for orally delivering osmotically subcoated microparticles containing at least one drug in admixture with a fluid. The method involves inserting osmotically subcoated microparticles into a hollow drug delivery chamber of a drug delivery device. The chamber has a first end and a second end. The first end of the chamber has a fluid passing drug formulation retainer. The drug delivery device has a first and second end. The first end of the drug delivering device is inserted into a fluid and the second end is inserted into the mouth of a patient. The patient then applies suction to the second end of the device to cause delivery of the fluid and microparticles into the patient's mouth.

The term “drug formulation retainer” as used herein, refers to a valve, plug or restriction, or the like that prevents passage of the drug formulation from the device. By “fluid passing drug formulation retainer” is intended a valve, plug or restriction or the like that allows for passage of fluids but does not allow for passage of other ingredients such as the drug formulation that is contained in the delivery device.

The dispensing device of this embodiment of the invention finds use where it is inconvenient or unsafe to use solid oral dosage forms such as capsules or tablets. The devices can be particularly useful in geriatric or pediatric patient populations but they can also be useful for those who have difficulty swallowing capsules or tablets. A single delivery device or several devices can be administered to a patient during a therapeutic program.

Generally the device is in prepared form prior to placement in a fluid. In at least one embodiment the dispensing device comprises a hollow drug formulation chamber with a first end and a second end. Contained within the chamber are drug formulation and fluid passing drug formulation retainers. The fluid passing drug formulation retainer comprises a restriction and a one-way plug. The diameter of the opening is smaller than the plug. In at least one embodiment the restriction is made by crimping an end of the chamber. The second end of the chamber has a drug formulation retainer for preventing release of the plug. In at least one embodiment the retainer is prepared by crimping the end of the chamber. Osmotically subcoated microparticles containing at least one drug are then placed in the chamber. An end-cap is placed over the second end of the chamber prior to use to prevent release of the drug formulation. In prepared form, the plug substantially seals the first end of the chamber, thereby preventing loss of the drug formulation from the first end.

The device can be formed from any suitable material that is physically and/or chemically compatible with both the drug and the liquid diluent to be mixed therein. In certain embodiments, representative materials for forming devices including the drug formulation chamber, the elongated tubular member, the end caps and tabs, include, without limitation, paper, plastic such as propylene/styrene copolymers, polyproylene, high density polyethylene, low density polyethylene and combinations thereof. The devices can have an inner diameter of between about 3 mm and about 8 mm, for example about 4 mm, about 5 mm, about 6 mm or about 7 mm and a wall thickness of between about 0.1 mm and about 0.4 mm, for example about 0.2 mm or about 0.3 mm. The devices can be between about 10 cm and about 30 cm in length, for example about 15 cm, about 20 cm, or about 25 cm in length.

The fluid passing drug formulation retainer permits the free flow of liquid medium but prohibits passage of the drug formulation from the device prior to delivery. Where the retainer comprises a one-way plug or valve, the plug or valve will seal the straw at atmospheric pressure. When suction is applied, fluid will be drawn around the plug and into the drug formulation chamber. Further, the plug has a density of less than one so that it will ascend to the top as the drug formulation is delivered into the oral cavity. When suction is no longer applied, the plug will remain in the highest position it reached during sipping. The plug can be prepared from closed cell polyethylene foam such as ETHAFOAM®. Other forms of one way plugs can be a balloon of elastomeric material, a one-way mechanical ball valve and the like.

Examples of fluid that can be used for suspending the drug formulation by sipping through the drug formulation chamber include any palatable liquid such as water, juice, milk, soda, coffee, tea etc. Care must be taken to ensure compatibility of the fluid with the drug formulation.

In at least one embodiment, a dose sipping delivery device according to the present invention can be prepared as follows. Jumbo size straws with an inside diameter of, for example, about 0.21 inches and a length of, for example, about 8 inches, are heat sealed at one end. The seal is partially cut off so that the “one-way” plug cannot escape. The partially sealed end is enclosed by half of a size 1 hard gelatin capsule. Osmotically subcoated microparticles are then placed inside the open end of the straw. A “one-way” plug made of closed cell polyethylene foam, MICROFOAM® (DuPont) is trimmed to snugly fit inside the straw. The plug is then placed inside the straw, on top of the microparticles. During operation, the plug end of the straw is placed into a glass of water and the protective gelatin capsule on the top of the straw is removed. By slowly applying suction through the partially sealed end of the straw, the microparticles are sucked into the mouth and easily swallowed.

While only specific combinations of the various features and components of the present invention have been discussed herein, it will be apparent to those of skill in the art that desired subsets of the disclosed features and components and/or alternative combinations of these features and components can be utilized as desired.

The above-described embodiments of the invention are intended to be examples of the present invention and alterations and modifications may be effected thereto by those of skill in the art without departing from the scope of the invention.

As will be seen from the non-limiting examples described below, the coatings of the invention are quite versatile. For example, the length and time for the lagtime can be controlled by the rate of hydration and the thickness of the modified release overcoat. The drug release rate subsequent to the lagtime can be determined at least in part by the thickness and permeability of the hydrated overcoat, and the level of osmotic agent in the osmotic subcoat. Thus, it is possible to regulate the rate of hydration and permeability of the overcoat and the level of osmotic agent in the osmotic subcoat so that the desired modified release drug profile can be achieved. There is no general preferred overcoat thickness, as this will depend on the drug being used in the core and also the modified release profile desired. Other parameters in combination with the thickness of the overcoat and the level of osmotic agent include varying the concentrations of some of the ingredients of the overcoat composition of the invention described and/or varying the curing temperature and length of curing the coated microparticles. The skilled artisan will know which parameters or combination of parameters to change for a desired modified release profile.

As exemplified in the examples below, dissolution profiles of the present invention are from quality control assays conducted according to instructions found in the United States Pharmacopoeia.

In the examples, where the percent of drug dissolved (i.e., % dissolved—defined as the percent of total drug in the composition dissolved into the external environment of use) data is presented, the data was obtained by taking a aliquot of the external environment of use (a 7 mL aliquot) at a time point measured from when the composition containing the drug was placed in the external environment of use (for example, at 2, 4, 8, 12, 14, 18, or 24 hours from when the composition was placed into the external environment of use) and HPLC (High Performance Liquid Chromatography) and a known standard were used to determine the concentration of the drug dissolved in the aliquot. The amount of drug dissolved in the aliquot, in turn, allowed for calculation of the drug dissolved in the external environment of use, using, at least, the following calculation at each time point:

Amount of drug per dissolution vessel at each time point:

$\frac{{A}_{sam} \times {C}_{std} \times VV}{{A}_{std}}$

| Abbreviation | Explanation | Units |
|---|---|---|
| Aₛₐₘ | Sample Response | μV/sec |
| A_{std} | Standard Response | μV/sec |
| C_{std} | Standard Concentration | mg/mL |
| VV | Vessel Volume | mL |

In certain embodiments of the examples, the osmotic subcoat and/or the outer coat contained substantially no water and/or no water.

### EXAMPLE 1

Pramipexole microparticle cores (2.5% drug loading)

| | |
|---|---|
| Pramipexole dihydrochloride | 50 g |
| Glyceryl monostearate (DMG 03VF) | 1950 g |

| Component | Function | Amount used (%) |
|---|---|---|
| Glyceryl monostearate | Spheronizing Agent | 97.5 |
| (DMG 03VF) | | |
| Pramipexole | Drug | 2.5 |
| dihydrochloride | | |

### EXAMPLE 2

Diltiazem microparticle cores (60% drug loading)

| | |
|---|---|
| Diltiazem hydrochloride | 1200 g |
| Glyceryl monostearate (DMG 03VF) | 800 g |

| Component | Function | Amount used (%) |
|---|---|---|
| Glyceryl monostearate | Spheronizing Agent | 40 |
| (DMG 03VF) | | |
| Diltiazem hydrochloride | Drug | 60 |

### EXAMPLE 3

Rivastigmine microparticle cores (10% drug loading)

| | |
|---|---|
| Rivastigmine tartrate | 60 g |
| Glyceryl monostearate (DMG 03VF) | 540 g |

| Component | Function | Amount used (%) |
|---|---|---|
| Glyceryl monostearate | Spheronizing Agent | 90 |
| (DMG 03VF) | | |
| Rivastigmine | Drug | 10 |

### EXAMPLE 4

Osmotic Subcoat

| Component | Function | Amount (%) |
|---|---|---|
| PHARMACOAT ® 606 | Osmotic deposition | 7.0 |
| | vehicle | |
| NaCl | Osmotic agent | 1.05 |
| Water | Medium | 91.95 |
| Weight Gain over the | N/A | 4.6 |
| uncoated microparticle | | |
| core | | |

### EXAMPLE 5

Modified Release Overcoat

| Component | Function | Amount (%) |
|---|---|---|
| Polyacrylate dispersion | Modified release | 75.0 |
| (e.g. EUDRAGIT ® | polymer | |
| NE30D) | | |
| Simethicone C | Anti-foaming agent | 0.15 |
| TWEEN ® 80 | Emulsifying agent | 0.03 |
| Magnesium Stearate | Lubricant | 1.5 |
| Talc | Lubricant | 1.5 |
| Water | Medium | 21.82 |
| Weight Gain over the | N/A | 35 |
| microparticle core | | |

### EXAMPLE 6

Modified Release Overcoat

| Component | Function | Amount (%) |
|---|---|---|
| Ethyl cellulose aqueous | Modified release | 60.0 |
| dispersion (e.g. | polymer | |
| SURELEASE ®) | | |
| Water | Medium | 40.0 |
| Weight Gain over the | N/A | 20.0 |
| microparticle core | | |

### EXAMPLE 7

Additional overcoat containing a drug

| Component | Function | Amount (%) |
|---|---|---|
| Rivastigmine | Drug | 8.33 |
| PHARMACOAT ® 606G | Low viscosity | 4.00 |
| | polymer | |
| Talc | Lubricant | 1.00 |
| Water | Medium | 86.67 |
| Weight Gain over the | N/A | 20.0 |
| microparticle core | | |

### EXAMPLE 8

### Pramipexole Dissolution Data

In certain embodiments, an amount of microspheres equivalent to 0.375 mg Pramipexole was sprinkled onto the media surface.

Dissolution Method: The dissolution tests were performed using a paddle apparatus (USP II, pH 6.8 Phosphate buffer) with a rotation of 50 rpm.

The external environment of use includes:
a dissolution medium,
the temperature of the dissolution medium was 37° C.+/−0.5° C.,
the volume of the dissolution medium was 500 ml,
the dissolution medium was a phosphate buffer having a pH of 6.8, and
the pressure of the atmosphere on the dissolution medium was 1 atmosphere.

See FIG. 1: Dissolution profiles of Pramipexole modified release microparticles with and without an osmotic agent (NaCl) in the subcoat.

Dissolution data:

| | With NaCl in Osmotic Subcoat | Without NaCl |
|---|---|---|
| Time (hours) | (% Dissolved) | (% Dissolved) |
| 2 | 8 | 6 |
| 4 | 24 | 13 |
| 6 | 47 | 22 |
| 8 | 67 | 31 |
| 12 | 86 | 48 |
| 18 | 94 | 66 |

FIG. 1 illustrates the effect of the osmotic agent in the osmotic subcoat on the rate and extent of drug release from Pramipexole modified release microparticles. Higher rate and extent of drug release (increased release and substantially full release) were observed with the Pramipexole modified release microparticles that had an osmotic subcoat containing an osmotic agent (NaCl), when compared to those without an osmotic agent in the subcoat.

See FIG. 2: Dissolution profiles of Pramipexole modified release microparticles in different dissolution media.

The external environment of use includes:
a dissolution medium,
the temperature of the dissolution medium was 37° C.+/−0.5° C.,
the volume of the dissolution medium was 500 ml,
the dissolution medium was a phosphate buffer having a pH of 6.8, or a phosphate buffer having a pH of 6.8 with 15.7 g NaCl added per Litre of buffer, and
the pressure of the atmosphere on the dissolution medium was 1 atmosphere.

Dissolution Method: The dissolution tests were performed using a paddle apparatus (USP II, pH 6.8 Phosphate buffer with and without added NaCl with a rotation of 50 rpm.

Dissolution data:

| | USP II, pH 6.8 | |
|---|---|---|
| | Phosphate buffer with | |
| | added NaCl* | USP II, pH 6.8 Phosphate buffer |
| Time (hours) | (% Dissolved) | (% Dissolved) |
| 2 | 6 | 15 |
| 4 | 33 | 63 |
| 6 | 59 | 92 |
| 8 | 76 | 101 |
| 12 | 92 | 107 |
| 18 | 98 | 110 |

*15.7 g NaCl per Litre

FIG. 2 compares release profiles of Pramipexole modified release microparticles in different dissolution media. The results showed a lower release (in terms of both rate and extent of drug release) of Pramipexole in the dissolution medium with added NaCl, as compared to the same Pramipexole modified release microparticles in dissolution medium without added NaCl. This demonstrates that drug release from coated microparticles is influenced by an osmotic gradient between the microparticle core and the external environment of use. The extra NaCl in the dissolution medium reduced the concentration differential between the outside medium and the osmotic subcoated microparticle, and thus reduced the osmotic pressure.

### EXAMPLE 9

### Diltiazem Dissolution Data:

Dissolution method: The dissolution tests were performed using a paddle apparatus (USP II, water) with a rotation of 100 rpm.

In certain embodiments, an amount of microspheres equivalent to 360 mg Diltiazem was sprinkled onto the media surface.

The external environment of use includes:
a dissolution medium,
the temperature of the dissolution medium was 37° C.+/−0.5° C.,
the volume of the dissolution medium was 900 ml,
the dissolution medium was water, and
the pressure of the atmosphere on the dissolution medium was 1 atmosphere.

See FIG. 3: Dissolution profiles of Diltiazem modified release microparticles with and without an osmotic subcoat.

Dissolution data:

| | With Osmotic Subcoat | Without Osmotic Subcoat |
|---|---|---|
| Time (hours) | (% Dissolved) | (% Dissolved) |
| 2 | 19 | N/A |
| 4 | 69 | N/A |
| 8 | 95 | 35 |
| 12 | 99 | N/A |
| 14 | 101 | 66 |
| 18 | 100 | N/A |
| 24 | 100 | 83 |

FIG. 3 illustrates the effect of the osmotic subcoat on the rate and extent of drug release from Diltiazem modified release microparticles. The results showed a higher rate and extent of drug release (increased release and substantially full release) of the Diltiazem modified release microparticles that had an osmotic subcoat containing an osmotic agent (NaCl), as compared to an otherwise similar composition without an osmotic subcoat.

### EXAMPLE 10

### Rivastigmine Dissolution Data:

Dissolution method: The dissolution tests were performed using a paddle apparatus (USP II) with a rotation of 50 rpm. Dissolution media used were 0.1N HCl; pH 4.5 acetate buffer and pH 6.8 phosphate buffer.

In certain embodiments, an amount of microspheres equivalent to 19.2 mg Rivastigmine Tartrate was sprinkled onto the media surface

The external environment of use includes:
a dissolution medium,
the temperature of the dissolution medium was 37° C.+/−0.5° C.,
the volume of the dissolution medium was 500 ml,
the dissolution medium was a 0.1 N HCl solution, a pH 4.5 acetate buffer solution, or a pH 6.8 phosphate buffer solution, and
the pressure of the atmosphere on the dissolution medium was 1 atmosphere.

See FIG. 4: Lagtime achieved from Rivastigmine delayed release microparticles in different dissolution media

Dissolution data:

| | | pH 4.5 acetate | |
|---|---|---|---|
| Time | 0.1N HCl | buffer | pH 6.8 phosphate buffer |
| (hours) | (% Dissolved) | (% Dissolved) | (% Dissolved) |
| 2 | 0 | 0 | 0 |
| 4 | 0 | 1 | 0 |
| 6 | 0 | 1 | 2 |
| 8 | 1 | 2 | 2 |
| 12 | 18 | 32 | 29 |
| 18 | 68 | 88 | 78 |
| 24 | 90 | 100 | 95 |

FIG. 4 illustrates lagtimes achieved from Rivastigmine delayed release microparticles in different dissolution media. The results demonstrate that lagtimes of the delayed release microparticles are substantially independent of the pH of the dissolution media.

See FIG. 5: Effect of NaCl level in the osmotic subcoat on dissolution profiles of Rivastigmine modified release microparticles.

The dissolution tests were performed using a paddle apparatus (USP II, 0.1N HCl) with a rotation of 50 rpm.

For the data associated with FIGS. 4-11, the amount of dissolution medium, in each case, was 500 ml and the temperature of the dissolution medium was, in each case, 37° C.+/−0.5° C.

The external environment of use includes:
a dissolution medium,
the temperature of the dissolution medium was 37° C.+/−0.5° C.,
the volume of the dissolution medium was 500 ml,
the dissolution medium was a 0.1 N HCl solution, and
the pressure of the atmosphere on the dissolution medium was 1 atmosphere.

Dissolution data:

| | % Dissolved | | | |
|---|---|---|---|---|
| | (15% NaCl in the osmotic | | (25% NaCl in the osmotic | |
| | subcoat) | | subcoat) | |
| Time | 25% weight | 35% weight | 25% weight | 35% weight |
| (hours) | gain | gain | gain | gain |
| 2 | 0 | 0 | 1 | 0 |
| 4 | 8 | 1 | 25 | 2 |
| 6 | 47 | 9 | 66 | 22 |
| 8 | 72 | 38 | 84 | 54 |
| 10 | 85 | 65 | 92 | 75 |
| 12 | 91 | 80 | 95 | 86 |
| 18 | 97 | 94 | 99 | 96 |
| 24 | 99 | 98 | 99 | 99 |

FIG. 5 illustrates the effect of the level of osmotic agent (NaCl) on the release profiles of Rivastigmine modified release microparticles. A faster release of Rivastigmine was seen in compositions that had a higher level of osmotic agent (NaCl) when compared to an otherwise similar composition that had a lower level of osmotic agent (regardless of weight gain).

See FIG. 6: Release profiles of NaCl and drug from Rivastigmine modified release microparticles.

Dissolution data:

| | Drug | NaCl |
|---|---|---|
| Time (hours) | (% Dissolved) | (% Dissolved) |
| 2 | 0 | 3 |
| 4 | 16 | 19 |
| 6 | 52 | N/A |
| 8 | 75 | 82 |
| 10 | 87 | 94 |
| 12 | 94 | 98 |
| 18 | 101 | 99 |
| 24 | 102 | 100 |

FIG. 6 compares release profiles of the osmotic agent (NaCl) and active drug from Rivastigmine modified release microparticles. The results demonstrate that the osmotic agent and the drug are released in a synchronised manner.

See FIG. 7: Pulsatile release of Rivastigmine from MODS beads

Dissolution data:

| | Time Zero |
|---|---|
| Time (hours) | (% Dissolved) |
| 2 | 55 |
| 4 | 56 |
| 6 | 58 |
| 8 | 64 |
| 10 | 71 |
| 12 | 78 |
| 18 | 90 |
| 24 | 95 |

FIG. 7 illustrates a pulsatile release profile of Rivastigmine from Rivastigmine delayed release microparticles coated with an additional overcoat containing Rivastigmine.

See FIG. 8: Drug release from Rivastigmine modified release microparticles (wherein modified release polymer is SURELEASE®) with osmotic subcoat vs. without osmotic subcoat

The dissolution tests were performed using a paddle apparatus (USP II, 0.1N HCl with added Sodium Chloride—14 g NaCl/Litre) with a rotation of 50 rpm.

The external environment of use includes:
a dissolution medium,
the temperature of the dissolution medium was 37° C.+/−0.5° C.,
the volume of the dissolution medium was 500 ml,
the dissolution medium was a 0.1 N HCl solution with sodium chloride added in an amount of 14 g NaCl per liter of solution, a pH 4.5 acetate buffer solution, or a pH 6.8 phosphate buffer solution, and
the pressure of the atmosphere on the dissolution medium was 1 atmosphere.

Dissolution data:

| | With subcoat | Without subcoat |
|---|---|---|
| Time (hours) | (% Dissolved) | (% Dissolved) |
| 2 | 1 | 3 |
| 4 | 6 | 6 |
| 6 | 19 | 7 |
| 8 | 36 | 9 |
| 10 | 50 | 11 |
| 12 | 60 | 12 |
| 18 | 78 | 14 |
| 24 | 88 | 16 |

FIG. 8 illustrates the effect of the osmotic subcoat on the rate and extent of drug release from Rivastigmine modified release microparticles, wherein the modified release polymer is SURELEASE®.

See FIG. 9: Dissolution profiles of Rivastigmine modified release microparticles (wherein modified release polymer is EUDRAGIT® NE30D) with and without an osmotic subcoat.

Dissolution data:

| | With Osmotic Subcoat | Without Osmotic Subcoat |
|---|---|---|
| Time (hours) | (% Dissolved) | (% Dissolved) |
| 2 | 0 | 0 |
| 4 | 1 | 2 |
| 6 | 9 | 10 |
| 8 | 37 | 27 |
| 12 | 79 | 57 |
| 18 | 98 | 77 |
| 24 | 100 | 86 |

FIG. 9 illustrates the effect of the osmotic subcoat on the rate and extent of drug release from Rivastigmine modified release microparticles, wherein the modified release polymer is EUDRAGIT® NE30D. The results showed a higher rate and extent of drug release (increased release and substantially full release) of the Rivastigmine modified release microparticles that had an osmotic subcoat containing an osmotic agent (NaCl), as compared to an otherwise similar composition without an osmotic subcoat.

See FIG. 10: Pulsatile drug release from Rivastigmine delayed release microparticles having an additional overcoat containing Rivastigmine—Dissolution data at time zero and after 1 month at 25° C./60% RH

Dissolution data:

| | | 1 month stored at |
|---|---|---|
| | Time Zero | 25° C./60% RH |
| Time (hours) | (% Dissolved) | (% Dissolved) |
| 2 | 55 | 55 |
| 4 | 56 | 56 |
| 6 | 58 | 59 |
| 8 | 64 | 65 |
| 10 | 71 | 72 |
| 12 | 78 | 78 |
| 18 | 90 | 89 |
| 24 | 95 | 95 |

FIG. 10 illustrates the stability of the Rivastigmine delayed release microparticles having an additional overcoat containing Rivastigmine. Pulsatile drug release is shown where approximately half of the drug was released immediately from an additional overcoat containing Rivastigmine, followed by a second phase of drug release from the microparticle core starting at approximately the 6 hour timepoint (lag time of 6 hours). The results demonstrate that there is no significant difference in the dissolution profiles at time zero and after 1 month storage at 25° C./60% RH.

See FIG. 11: Pulsatile drug release from mixture of Rivastigmine immediate release microparticles and Rivastigmine delayed release microparticles—Stability data at time zero and after 1 month at 25° C./60% RH

Dissolution data:

| | | 1 month stored at |
|---|---|---|
| | Time Zero | 25° C./60% RH |
| Time (hours) | (% Dissolved) | (% Dissolved) |
| 2 | 50 | 52 |
| 4 | 51 | 55 |
| 6 | 55 | 59 |
| 8 | 64 | 66 |
| 10 | 73 | 74 |
| 12 | 80 | 82 |
| 18 | 90 | 94 |
| 24 | 94 | 99 |

FIG. 11 illustrates the stability of a dosage form containing a mixture of Rivastigmine immediate release microparticles and Rivastigmine delayed release microparticles. Pulsatile drug release is shown where approximately half of the drug was released immediately from the immediate release microparticles, followed by a second phase of drug release from the delayed release microparticles starting at approximately the 6 hour timepoint (lag time of 6 hours). The results demonstrate that there is no significant difference in the dissolution profiles at time zero and after 1 month storage at 25° C./60% RH.

TABLE 1
Polyethoxylated Fatty Acids
Although polyethylene glycol (PEG) itself does not function as a
surfactant, a variety of PEG-fatty acid esters have useful surfactant
properties. Examples of polyethoxylated fatty acid monoester
surfactants commercially available
are shown here in Table 1.
PEG-Fatty Acid Monoester Surfactants

| Compound | Commercial Product (Supplier) | HLB |
|---|---|---|
| PEG 4-100 | Crodet L series (Croda) | >9 |
| monolaurate | | |
| PEG 4-100 | Crodet O series (Croda) | >8 |
| monooleate | | |
| PEG 4-100 | Crodet S series (Croda), Myrj Series | >6 |
| monostearate | (Atlas/ICI) | |
| PEG 400 distearate | Cithrol 4DS series (Croda) | >10 |
| PEG 100, 200, 300 | Cithrol ML series (Croda) | >10 |
| monolaurate | | |
| PEG 100, 200, 300 | Cithrol MO series (Croda) | >10 |
| monooleate | | |
| PEG 400 dioleate | Cithrol 4DO series (Croda) | >10 |
| PEG 400-1000 | Cithrol MS series (Croda) | >10 |
| monostearate | | |
| PEG-1 stearate | Nikkol MYS-IEX (Nikko), Coster KI | 2 |
| | (Condea) | |
| PEG-2 stearate | Nikkol MYS-2 (Nikko) | 4 |
| PEG-2 oleate | NikkoI MYO-2 (Nikko) | 4.5 |
| PEG-4 laurate | Mapeg ® 200 ML (PPG), | 9.3 |
| | Kessco ® PEG 200ML (Stepan), | |
| | LIPOPEG 2L (LIPO Chem.) | |
| PEG-4 oleate | Mapeg ® 200 MO (PPG), | 8.3 |
| | Kessco ® PEG200 MO (Stepan), | |
| PEG-4 stearate | Kessco ® PEG 200 MS (Stepan), | 6.5 |
| | Hodag 20 S (Calgene), Nikkol MYS-4 | |
| | (Nikko) | |
| PEG-5 stearate | Nikkol TMGS-5 (Nikko) | 9.5 |
| PEG-5 oleate | Nikkol TMGO-5 (Nikko) | 9.5 |
| PEG-6 oleate | Algon OL 60 (Auschem SpA), | 8.5 |
| | Kessco ® PEG 300 MO (Stepan), | |
| | Nikkol MYO-6 (Nikko), | |
| | Emulgante A6 (Condea) | |
| PEG-7 oleate | Algon OL 70 (Auschem SpA) | 10.4 |
| PEG-6 laurate | Kessco ® PEG300 ML (Stepan) | 11.4 |
| PEG-7 laurate | Lauridac 7 (Condea) | 13 |
| PEG-6 stearate | Kessco ® PEG300 MS (Stepan) | 9.7 |
| PEG-8 laurate | Mapeg ® 400 ML (PPG), | 13 |
| | LIPOPEG 4DL(Lipo Chem.) | |
| PEG-8 oleate | Mapeg ® 400 MO (PPG), | 12 |
| | Emulgante A8 (Condea); | |
| | Kessco PEG 400 MO (Stepan) | |
| PEG-8 stearate | Mapeg ® 400 MS (PPG), Myrj 45 | 12 |
| PEG-9 oleate | Emulgante A9 (Condea) | >10 |
| PEG-9 stearate | Cremophor 59 (BASF) | >10 |
| PEG-10 laurate | Nikkol MYL-10 (Nikko), Lauridac 10 | 13 |
| | (Croda) | |
| PEG-10 oleate | Nikkol MYO-10 (Nikko) | 11 |
| PEG-10 stearate | Nikkol MYS-10 (Nikko), Coster K100 | 11 |
| | (Condea) | |
| PEG-12 laurate | Kessco ® PEG 600ML (Stepan) | 15 |
| PEG-12 oleate | Kessco ® PEG 600MO (Stepan) | 14 |
| PEG-12 ricinoleate | (CAS #9004-97-1) | >10 |
| PEG-12 stearate | Mapeg ® 600 MS (PPG), | 14 |
| | Kessco ® PEG 600MS (Stepan) | |
| PEG-15 stearate | Nikkol TMGS-15 (Nikko), Koster K15 | 14 |
| | (Condea) | |
| PEG-15 oleate | Nikkol TMGO- 15 (Nikko) | 15 |
| PEG-20 laurate | Kessco ® PEG 1000 ML (Stepan) | 17 |
| PEG-20 oleate | Kessco ® PEG 1000 MO (Stepan) | 15 |
| PEG-20 stearate | Mapeg ® 1000 MS (PPG), | 16 |
| | Kessco ® PEG 1000 | |
| | MS (Stepan), Myrj 49 | |
| PEG-25 stearate | Nikkol MYS-25 (Nikko) | 15 |
| PEG-32 laurate | Kessco ® PEG 1540 ML (Stepan) | 16 |
| PEG-32 oleate | Kessco ® PEG 1540 MO (Stepan) | 17 |
| PEG-32 stearate | Kessco ® PEG 1540 MS (Stepan) | 17 |
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-40 stearate | Myrj 52, Emerest ® 2715 (Henkel), | >10 |
| | Nikkol MYS-40 (Nikko) | |
| PEG-45 stearate | Nikkol MYS-45 (Nikko) | 18 |
| PEG-50 stearate | Myrj 53 | >10 |
| PEG-55 stearate | Nikkol MYS-55 (Nikko) | 18 |
| PEG-100 oleate | Crodet 0-100 (Croda) | 18.8 |
| PEG-100 stearate | Myrj 59, Arlacel 165 (ICI) | 19 |
| PEG-200 oleate | Albunol 200 MO (Taiwan Surf.) | >10 |
| PEG-400 oleate | LACTOMUL (Henkel), Albunol 400 MO | >10 |
| | (Taiwan Surf.) | |
| PEG-600 oleate | Albunol 600 MO (Taiwan Surf) | >10 |

TABLE 2
PEG-Fatty Acid Diesters
Polyethylene glycol (PEG) fatty acid diesters are also suitable for use as
surfactants in the compositions of the present invention. Representative
PEG-fatty acid diesters are shown here in Table 2.
PEG-Fatty Acid Diester Surfactants

| Compound | Commercial Product (Supplier) | HLB |
|---|---|---|
| PEG-4 dilaurate | Mapeg ® 200 DL (PPG), | 7 |
| | Kessco ® PEG 200 DL (Stepan), | 6 |
| | LIPOPEG 2-DL (Lipo Chem.) | |
| PEG-4 dioleate | Mapeg ® 200 DO (PPG), | 6 |
| PEG-4 distearate | Kessco ® 200 DS (Stepan) | 5 |
| PEG-6 dilaurate | Kessco ® PEG 300 DL (Stepan) | 9.8 |
| PEG-6 dioleate | Kessco ® PEG 300 DO (Stepan) | 7.2 |
| PEG-6 distearate | Kessco ® PEG 300 DS (Stepan) | 6.5 |
| PEG-8 dilaurate | Mapeg ® 400 DL (PPG), | 11 |
| | Kessco ® PEG 400 DL | |
| | (Stepan), LIPOPEG 4 DL (Lipo Chem.) | |
| PEG-8 dioleate | Mapeg ® 400 DO (PPG), | 8.8 |
| | Kessco ® PEG 400 DO | |
| | (Stepan), LIPOPEG 4 DO (Lipo Chem.) | |
| PEG-8 distearate | Mapeg ® 400 DS (PPG), CDS 400 (Nikkol) | 11 |
| PEG-10 dipalmitate | Polyaldo 2PKFG | >10 |
| PEG-12 dilaurate | Kessco ® PEG 600 DL (Stepan) | 11.7 |
| PEG-12 distearate | Kessco ® PEG 600 DS (Stepan) | 10.7 |
| PEG-12 dioleate | Mapeg ® 600 DO (PPG), | 10 |
| | Kessco ® 600 DO(Stepan) | |
| PEG-20 dilaurate | Kessco ® PEG 1000 DL (Stepan) | 15 |
| PEG-20 dioleate | Kessco ® PEG 1000 DO (Stepan) | 13 |
| PEG-20 distearate | Kessco ® PEG 1000 DS (Stepan) | 12 |
| PEG-32 dilaurate | Kessco ® PEG 1540 DL (Stepan) | 16 |
| PEG-32 dioleate | Kessco ® PEG 1540 DO (Stepan) | 15 |
| PEG-32 distearate | Kessco ® PEG 1540 DS (Stepan) | 15 |
| PEG-400 dioleate | Cithrol 4DO series (Croda) | >10 |
| PEG-400 distearate | Cithrol 4DS series (Croda) | >10 |

TABLE 3
PEG-Fatty Acid Mono- and Di-ester Mixtures
In general, mixtures of surfactants are also useful in the present invention,
including mixtures of two or more commercial surfactant products. Several
PEG-fatty acid esters are marketed commercially as mixtures or mono-
and diesters. Representative surfactant mixtures are shown here in Table 3.
PEG-Fatty Acid Mono-and Diester Mixtures

| Compound | Commercial Product (Supplier) |
|---|---|
| PEG 4-150 mono, | Kessco ® PEG 200-6000 mono, dilaurate (Stepan) |
| dilaurate | |
| PEG 4-150 mono, | Kessco ® PEG 200-6000 mono, dioteate (Stepan) |
| dioleate | |
| PEG 4-150 mono, | Kessco ® 200-6000 mono, distearate (Stepan) |
| distearate | |

TABLE 4
Polyethylene Glycol Glycerol Fatty Acid Esters
Suitable PEG glycerol fatty acid esters are shown here in Table 4.
PEG Glycerol Fatty Acid Esters

| Compound | Commercial Product (Supplier) | HLB |
|---|---|---|
| PEG-20 glyceryl | Tagat ® L (Goldschmidt) | 16 |
| laurate | | |
| PEG-30 glyceryl | Tagat ® L2 (Goldschmidt) | 16 |
| laurate | | |
| PEG-15 glyceryl | Glycerox L series (Croda) | 15 |
| laurate | | |
| PEG-40 glyceryl | Glycerox L series (Croda) | 15 |
| laurate | | |
| PEG-20 glyceryl | Capmul ® EMG (ABITEC), | 13 |
| stearate | Aldo ® MS-20 KFG (Lonza) | |
| PEG-20 glyceryl | Tagat ® O (Goldschmidt) | >10 |
| oleate | | |
| PEG-30 glyceryl | Tagat ® O2 (Goldschmidt) | >10 |
| oleate | | |

TABLE 5
Alcohol--Oil Transesterification Products
A large number of surfactants of different degrees of lipophilicity or
hydrophilicity can be prepared by reaction of alcohols or polyalcohols
with a variety of natural and/or hydrogenated oils. In certain embodiments,
the oils used are castor oil or hydrogenated castor oil or an edible
vegetable oil such as corn oil, olive oil, peanut oil, palm kernel oil,
apricot kernel oil, or almond oil. Examples of alcohols include glycerol,
propylene glycol, ethylene glycol, polyethylene glycol, sorbitol, and
pentaerythritol. Representative surfactants of this class suitable for
use in the present invention are shown here in Table 5.
Transesterification Products of Oils and Alcohols

| Compound | Commercial Product (Supplier) | HLB |
|---|---|---|
| PEG-3 castor oil | Nikkol CO-3 (Nikko) | 3 |
| PEG-5, 9, and 16 | ACCONON CA series (ABITEC) | 6-7 |
| castor oil | | |
| PEG-20 castor oil | Emalex C-20 (Nihon Emulsion), Nikkol | 11 |
| | CO-20 TX (Nikko) | |
| PEG-23 castor oil | Emulgante EL23 | >10 |
| PEG-30 castor oil | Emalex C-30 (Nihon Emulsion), | 11 |
| | Alkamuls ® EL 620 | |
| | (Rhone-Poulenc), Incrocas 30 (Croda) | |
| PEG-35 castor oil | Cremophor EL and EL-P (BASF), | |
| | Emulphor EL, Incrocas-35 | |
| | (Croda), Emulgin RO 35 (Henkel) | |
| PEG-38 castor oil | Emulgante EL 65 (Condea) | |
| PEG-40 castor oil | Emalex C-40 (Nihon Emulsion), | 13 |
| | Alkamuls ® EL 719 | |
| | (Rhone-Poulenc) | |
| PEG-50 castor oil | Emalex C-50 (Nihon Emulsion) | 14 |
| PEG-56 castor oil | Eumulgin ® PRT 56 (Pulcra SA) | >10 |
| PEG-60 castor oil | Nikkol CO-60TX (Nikko) | 14 |
| PEG-100 castor oil | Thornley | >10 |
| PEG-200 castor oil | Eumulgin ® PRT 200 (Pulcra SA) | >10 |
| PEG-5 hydrogenated | Nikkol HCO-5 (Nikko) | 6 |
| castor oil | | |
| PEG-7 hydrogenated | Simusol ® 989 (Seppic), Cremophor | 6 |
| castor oil | WO7 (BASF) | |
| PEG-10 hydrogenated | Nikkol HCO-10 (Nikko) | 6.5 |
| castor oil | | |
| PEG-20 hydrogenated | Nikkol HCO-20 (Nikko) | 11 |
| castor oil | | |
| PEG-25 hydrogenated | Simulsol ® 1292 (Seppic), Cerex | 11 |
| castor oil | ELS 250 (Auschem SpA) | |
| PEG-30 hydrogenated | Nikkol HCO-30 (Nikko) | 11 |
| castor oil | | |
| PEG-40 hydrogenated | Cremophor RH 40 (BASF), Croduret | 13 |
| castor oil | (Croda), Emulgin HRE 40 (Henkel) | |
| PEG-45 hydrogenated | Cerex ELS 450 (Auschem Spa) | 14 |
| castor oil | | |
| PEG-50 hydrogenated | Emalex HC-50 (Nihon Emulsion) | 14 |
| castor oil | | |
| PEG-60 hydrogenated | Nikkol HCO-60 (Nikko), Cremophor | 15 |
| castor oil | RH 60 (BASF) | |
| PEG-80 hydrogenated | Nikkol HCO-80 (Nikko) | 15 |
| castor oil | | |
| PEG-100 | Nikkol HCO-100 (Nikko) | 17 |
| hydrogenated castor | | |
| oil | | |
| PEG-6 corn oil | Labrafil ® M 2125 CS (Gattefosse) | 4 |
| PEG-6 almond oil | Labrafil ® M 1966 CS (Gattefosse) | 4 |
| PEG-6 apricot kernel | Labrafil ® M 1944 CS (Gattefosse) | 4 |
| oil | | |
| PEG-6 olive oil | Labrafil ® M 1980 CS (Gattefosse) | 4 |
| PEG-6 peanut oil | Labrafil ® M 1969 CS (Gattefosse) | 4 |
| PEG-6 hydrogenated | Labrafil ® M 2130 BS (Gattefosse) | 4 |
| palm kernel oil | | |
| PEG-6 palm kernel oil | Labrafil ® M 2130 CS (Gattefosse) | 4 |
| PEG-6 triolein | Labrafil ® M 2735 CS (Gattefosse) | 4 |
| PEG-8 corn oil | Labrafil ® WL 2609 BS (Gattefosse) | 6-7 |
| PEG-20 corn | Crovol M40 (Croda) | 10 |
| glycerides | | |
| PEG-20 almond | Crovol A40 (Croda) | 10 |
| glycerides | | |
| PEG-25 trioleate | TAGAT ® TO (Goldschmidt) | 11 |
| PEG-40 palm kernel | Crovol PK-70 | >10 |
| oil | | |
| PEG-60 corn | Crovol M70(Croda) | 15 |
| glycerides | | |
| PEG-60 almond | Crovol A70 (Croda) | 15 |
| glycerides | | |
| PEG-4 caprylic/capric | Labrafac ® Hydro (Gattefosse), | 4-5 |
| triglyceride | | |
| PEG-8 caprylic/capric | Labrasol (Gattefosse), Labrafac CM 10 | >10 |
| glycerides | (Gattefosse) | |
| PEG-6 caprylic/capric | SOFTIGEN ® 767 (Huls), | 19 |
| glycerides | Glycerox 767 (Croda) | |
| Lauroyl macrogol-32 | GELUCIRE 44/14 (Gattefosse) | 14 |
| glyceride | | |
| Stearoyl macrogol | GELUCIRE 50/13 (Gattefosse) | 13 |
| glyceride | | |
| Mono, di, tri, tetra | SorbitoGlyceride (Gattefosse) | <10 |
| esters of vegetable oils | | |
| and sorbitol | | |
| Pentaerythrityl | Crodamol PTIS (Croda) | <10 |
| tetraisostearate | | |
| Pentaerythrityl | Albunol DS (Taiwan Surf.) | <10 |
| distearate | | |
| Pentaerythrityl | Liponate PO-4 (Lipo Chem.) | <10 |
| tetraoleate | | |
| Pentaerythrityl | Liponate PS-4 (Lipo Chem.) | <10 |
| tetrastearate | | |
| Pentaerythrityl | Liponate PE-810 (Lipo Chem.), Crodamol | <10 |
| tetracaprylate/ | PTC (Croda) | |
| tetracaprate | | |
| Pentaerythrityl | Nikkol Pentarate 408 (Nikko) | |
| tetraoctanoate | | |

TABLE 6
Polyglycerized Fatty Acids
Polyglycerol esters of fatty acids are also suitable surfactants
for the present invention.
Examples of suitable polyglyceryl esters are shown here in Table 6.
Polyglycerized Fatty Acids

| Compound | Commercial Product (Supplier) | HLB |
|---|---|---|
| Polyglyceryl-2 stearate | Nikkol DGMS (Nikko) | 5-7 |
| Polyglyceryl-2 oleate | Nikkol DGMO (Nikko) | 5-7 |
| Polyglyceryl-2 | Nikkol DGMIS (Nikko) | 5-7 |
| isostearate | | |
| Polyglyceryl-3 oleate | Caprol ® 3G0 (ABITEC), Drewpol 3-1-O | 6.5 |
| | (Stepan) | |
| Polyglyceryl-4 oleate | Nikkol Tetraglyn 1-O (Nikko) | 5-7 |
| Polyglyceryl-4 stearate | Nikkol Tetraglyn 1-S (Nikko) | 5-6 |
| Polyglyceryl-6 oleate | Drewpol 6-1-O (Stepan), Nikkol | 9 |
| | Hexaglyn 1-O (Nikko) | |
| Polyglyceryl-10 | Nikkol Decaglyn 1-L (Nikko) | 15 |
| laurate | | |
| Polyglyceryl-10 oleate | Nikkol Decaglyn 1-O (Nikko) | 14 |
| Polyglyceryl-10 | Nikkol Decaglyn 1-S (Nikko) | 12 |
| stearate | | |
| Polyglyceryl-6 | Nikkol Hexaglyn PR-15 (Nikko) | |
| ricinoleate | | |
| Polyglyceryl-10 | Nikkol Decaglyn I-LN (Nikko) | 12 |
| linoleate | | |
| Polyglyceryl-6 | Nikkol Hexaglyn S-O (Nikko) | <10 |
| pentaoleate | | |
| Polyglyceryl-3 | Cremophor G032 (BASF) | <10 |
| dioleate | | |
| Polyglyceryl-3 | Cremophor GS32 (BASF) | <10 |
| distearate | | |
| Polyglyceryl-4 | Nikkol Tetraglyn 5-O (Nikko) | <10 |
| pentaoleate | | |
| Polyglyceryl-6 | Caprol ® 6G20 (ABITEC); Hodag | 8.5 |
| dioleate | PGO-62 (Calgene), PLUROL OLEIQUE | |
| | CC 497 (Gattefosse) | |
| Polyglyceryl-2 | Nikkol DGDO (Nikko) | 7 |
| dioleate | | |
| Polyglyceryl-10 | Nikkol Decaglyn 3-O (Nikko) | 7 |
| trioleate | | |
| Polyglyceryl-10 | Nikkol Decaglyn 5-O (Nikko) | 3.5 |
| pentaoleate | | |
| Polyglyceryl-10 | Nikkol Decagtyn 7-O (Nikko) | 3 |
| septaoleate | | |
| Polyglyceryl-10 | Caprol ® 10G40 (ABITEC); Hodag | 6.2 |
| tetraoleate | PGO-62 (CALGENE), Drewpol | |
| | 10-4-O (Stepan) | |
| Polyglyceryl-10 | Nikkol Decaglyn 10-IS (Nikko) | <10 |
| decaisostearate | | |
| Polyglyceryl-10 | Drewpol 10-10-O (Stepan), Caprol | 3.5 |
| decaoleate | 10G10O (ABITEC), Nikkol Decaglyn | |
| | 10-O | |
| Polyglyceryl-10 mono, | Caprol ® PGE 860 (ABITEC) | 11 |
| dioleate | | |
| Polyglyceryl | Polymuls (Henkel) | 3-20 |
| polyricinoIeate | | |

TABLE 7
Propylene Glycol Fatty Acid Esters
Esters of propylene glycol and fatty acids are suitable surfactants
for use in the present invention. Examples of surfactants
of this class are given here in Table 7.
Propylene Glycol Fatty Acid Esters

| Compound | Commercial Product (Supplier) | HLB |
|---|---|---|
| Propylene glycol | Capryol 90 (Gattefosse), Nikkol Sefsol 218 | <10 |
| monocaprylate | (Nikko) | |
| Propylene glycol | Lauroglycol 90 (Gattefosse), Lauroglycol | <10 |
| monolaurate | FCC (Gattefosse) | |
| Propylene glycol | Lutrol OP2000 (BASF) | <10 |
| oleate | | |
| Propylene glycol | Mirpyl | <10 |
| myristate | | |
| Propylene glycol | ADM PGME-03 (ADM), LIPO PGMS (Lipo | 3-4 |
| monostearate | Chem.), Aldo ® PGHMS (Lonza) | |
| Propylene glycol | | <10 |
| hydroxy stearate | | |
| Propylene glycol | PROPYMULS (Henkel) | <10 |
| ricinoleate | | |
| Propylene glycol | | <10 |
| isostearate | | |
| Propylene glycol | Myverol P-06 (Eastman) | <10 |
| monooleate | | |
| Propylene glycol | Captex ® 200 (ABITEC), Miglyol ® 840 | >6 |
| dicaprylate/dicaprate | (Huls), Neobee ® M-20 (Stepan) | |
| Propylene glycol | Captex ® 800 (ABITEC) | |
| dioctanoate | | |
| Propylene glycol | LABRAFAC | >6 |
| caprylate/caprate | PG (Gattefosse) | |
| Propylene glycol | | >6 |
| dilaurate | | |
| Propylene glycol | Kessco ® PGDS (Stepan) | >6 |
| distearate | | |
| Propylene glycol | Nikkol Sefsol 228 (Nikko) | >6 |
| dicaprylate | | |
| Propylene glycol | Nikkol PDD (Nikko) | >6 |
| dicaprate | | |

TABLE 8
Mixtures of Propylene Glycol Esters--Glycerol Esters
In general, mixtures of surfactants are also suitable for use in the
present invention. In particular, mixtures of propylene glycol fatty
acid esters and glycerol fatty acid esters are suitable and are
commercially available. Examples of these surfactants are
shown here in Table 8.
Glycerol/Propylene Glycol Fatty Acid Esters

| Compound | Commercial Product (Supplier) | HLB |
|---|---|---|
| Oleic | ATMOS 300, ARLACEL 186 (ICI) | 3-4 |
| Stearic | ATMOS 150 | 3-4 |

TABLE 9
Mono- and Diglycerides
Another class of surfactants is the class of mono- and diglycerides. These surfactants
are generally lipophilic. Examples of these surfactants are given here in Table 9.
Mono- and Diglyceride Surfactants

| Compound | Commercial Product (Supplier) | HLB |
|---|---|---|
| Monopalmitolein | (Larodan) | <10 |
| (C16:1) | | |
| Monoelaidin | (Larodan) | <10 |
| (C18:1) | | |
| Monocaproin | (Larodan) | <10 |
| (C6) | | |
| Monocaprylin | (Larodan) | <10 |
| Monocaprin | (Larodan) | <10 |
| Monolaurin | (Larodan) | <10 |
| Glyceryl | Nikkol MGM (Nikko) | 3-4 |
| monomyristate (C14) | | |
| Glyceryl monooleate | PECEOL (Gattefosse), Hodag GMO-D, Nikkol | 3-4 |
| (C18:1) | MGO (Nikko) | |
| Glyceryl monooleate | RYLO series (Danisco), DIMODAN series | 3-4 |
| | (Danisco), EMULDAN (Danisco), ALDO ® MOFG | |
| | (Lonza), Kessco GMO (Stepan), | |
| | MONOMULS ® series (Henkel), TEGIN O, | |
| | DREWMULSE GMO (Stepan), Atlas G-695 | |
| | (ICI), GMOrphic 80 (Eastman), ADM DMG-40, | |
| | 70, and 100 (ADM), Myverol (Eastman) | |
| Glycerol monooleate/ | OLICINE (Gattefosse) | 3-4 |
| linoleate | | |
| Glycerol | Maisine (Gattefosse), MYVEROL 18-92, | 3-4 |
| monolinoleate | Myverol 18-06 (Eastman) | |
| Glyceryl ricinoleate | Softigen ® 701 (Huls), HODAG GMR-D | 6 |
| | (Calgene), ALDO ® MR (Lonza) | |
| Glyceryl monolaurate | ALDO ® MLD (Lonza), Hodag GML (Calgene) | 6.8 |
| Glycerol | Emalex GMS-P (Nihon) | 4 |
| monopalmitate | | |
| Glycerol monostearate | Capmul ® GMS. (ABITEC), Myvaplex | 5-9 |
| | (Eastman), IMWITOR ® 191 (Huls), CUTINA | |
| | GMS, Aldo ® MS (Lonza), Nikkol MGS series | |
| | (Nikko) | |
| Glyceryl mono-, | Capmul ® GMO-K (ABITEC) | <10 |
| dioleate | | |
| Glyceryl | CUTINA MD-A, ESTAGEL-G18 | <10 |
| palmitic/stearic | | |
| Glyceryl acetate | Lamegin ® EE (Grunau GmbH) | <10 |
| Glyceryl laurate | Inwitor ® 312 (Huls), Monomuls ® 90-45 (Grunau | 4 |
| | GmbH), Aldo ® MLD (Lonza) | |
| Glyceryl citrate/ | Imwitor ® 375 (Huls) | <10 |
| lactate/oleate/linoieate | | |
| Glyceryl caprylate | Imwitor ® 308 (Huls), Capmul ® MCMC8 | 5-6 |
| | (ABITEC) | |
| Glyceryl | Capmul ® MCM (ABITEC) | 5-6 |
| caprylate/caprate | | |
| Caprylic acid mono, | Imwitor ® 988 (Huls) | 5-6 |
| diglycerides | | |
| Caprylic/capric | Imwitor ® 742 (Huls) | <10 |
| glycerides | | |
| Mono-and diacetylated | Myvacet ® 9-45, Myvacet ® 9-40, Myvacet ® 9-08 | 3.8-4 |
| monoglycerides | (Eastman), Lamegin ® (Grunau) | |
| Glyceryl monostearate | Aldo ® MS, Arlacel 129 (ICI), LIPO GMS (Lipo | 4.4 |
| | Chem.), Imwitor ® 191 (Huls), Myvaplex | |
| | (Eastman) | |
| Lactic acid esters of | LAMEGIN GLP (Henkel) | <10 |
| mono, diglycerides | | |
| Dicaproin (C6) | (Larodan) | <10 |
| Dicaprin (C10) | (Larodan) | <10 |
| Dioctanoin (C8) | (Larodan) | <10 |
| Dimyristin (C14) | (Larodan) | <10 |
| Dipalmitin (C16) | (Larodan) | |
| Distearin | (Larodan) | <10 |
| Glyceryl dilaurate | Capmul ® GDL (ABITEC) | 3-4 |
| (C12) | | |
| Glyceryl dioleate | Capmul ® GDO (ABITEC) | 3-4 |
| Glycerol esters of fatty | GELUCIRE 39/01 (Gattefosse), | 1 |
| acids | GELUCIRE 43/01 (Gattefosse) | |
| | GELUCIRE 37/06 (Gattefosse) | 6 |
| Dipalmitolein (C16:1) | (Larodan) | <10 |
| 1,2 and 1,3-diolein | | |
| (C18:1) | | |
| Dielaidin (C18:1) | (Larodan) | <10 |
| Dilinolein (C18:2) | (Larodan) | <10 |

TABLE 10
Sterol and Sterol Derivatives
Sterols and derivatives of sterols are suitable surfactants for use in the
present invention. These surfactants can be hydrophilic or lipophilic.
Examples of surfactants of this class are shown here in Table 10.
Sterol and Sterol Derivative Surfactants

| Compound | Commercial Product (Supplier) | HLB |
|---|---|---|
| Cholesterol, sitosterol, | | <10 |
| lanosterol | | |
| PEG-24 cholesterol | Solulan C-24 (Amerchol) | >10 |
| ether | | |
| PEG-30 cholestanol | Nikkol DHC (Nikko) | >10 |
| Phytosterol | GENEROL series (Henkel) | <10 |
| PEG-25 phyto sterol | Nikkol BPSH-25 (Nikko) | >10 |
| PEG-5 soya sterol | Nikkol BPS-S (Nikko) | <10 |
| PEG-10 soya sterol | Nikkol BPS-10 (Nikko) | <10 |
| PEG-20 soya sterol | Nikkol BPS-20 (Nikko) | <10 |
| PEG-30 soya sterol | Nikkol BPS-30 (Nikko) | >10 |

TABLE 11
Polyethylene Glycol Sorbitan Fatty Acid Esters
A variety of PEG-sorbitan fatty acid esters are available and are suitable
for use as surfactants in the present invention. In general, these
surfactants are hydrophilic, although several lipophilic surfactants
of this class can be used. Examples of these surfactants are
shown here in Table 11.
PEG-Sorbitan Fatty Acid Esters

| Compound | Commercial Product (Supplier) | HLB |
|---|---|---|
| PEG-10 sorbitan | Liposorb L-10 (Lipo Chem.) | >10 |
| laurate | | |
| PEG-20 sorbitan | Tween-20 (Atlas/ICI), Crillet 1 (Croda), | 17 |
| monolaurate | DACOL MLS 20 (Condea) | |
| PEG-4 sorbitan | Tween-21 (Atlas/ICI), Crillet 11 (Croda) | 13 |
| monolaurate | | |
| PEG-80 sorbitan | Hodag PSML-80 (Calgene); T-Maz 28 | >10 |
| monolaurate | | |
| PEG-6 sorbitan | Nikkol GL-1 (Nikko) | 16 |
| monolaurate | | |
| PEG-20 sorbitan | Tween-40 (Atlas/ICI), Crillet 2 (Croda) | 16 |
| monopalmitate | | |
| PEG-20 sorbitan | Tween-60 (Atlas/ICI), Crillet 3 (Croda) | 15 |
| monostearate | | |
| PEG-4 sorbitan | Tween-61 (Atlas/ICI), Crillet 31 (Croda) | 9.6 |
| monostearate | | |
| PEG-8 sorbitan | DACOL MSS (Condea) | >10 |
| monostearate | | |
| PBG-6 sorbitan | Nikkol TS106 (Nikko) | 11 |
| monostearate | | |
| PEG-20 sorbitan | Tween-65 (Atlas/ICI), Crillet 35 (Croda) | 11 |
| tristearate | | |
| PEG-6 sorbitan | Nikkol GS-6 (Nikko) | 3 |
| tetrastearate | | |
| PEG-60 sorbitan | Nikkol GS-460 (Nikko) | 13 |
| tetrastearate | | |
| PEG-5 sorbitan | Tween-81 (Atlas/ICI), Crillet 41 (Croda) | 10 |
| monooleate | | |
| PEG-6 sorbitan | Nikkol TO-106 (Nikko) | 10 |
| monooleate | | |
| PEG-20 sorbitan | Tween-80 (Atlas/ICI), Crillet 4 (Croda) | 15 |
| monooleate | | |
| PEG-40 sorbitan | Emalex ET 8040, (Nihon Emulsion) | 18 |
| oleate | | |
| PEG-20 sorbitan | Tween-85 (Atlas/ICI), Crillet 45 (Croda) | 11 |
| trioleate | | |
| PEG-6 sorbitan | Nikkol GO-4 (Nikko) | 8.5 |
| tetraoleate | | |
| PEG-30 sorbitan | Nikkol GO-430 (Nikko) | 12 |
| tetraoleate | | |
| PEG-40 sorbitan | Nikkol GO-440 (Nikko) | 13 |
| tetraoleate | | |
| PEG-20 sorbitan | Tween-120 (Atlas/ICI), Crillet 6 (Croda) | >10 |
| monoisostearate | | |
| PEG sorbitol | Atlas G-1086 (ICI) | 10 |
| hexaoleate | | |
| PEG-6 sorbitol | Nikkol GS-6 (Nikko) | 3 |
| hexastearate | | |

TABLE 12
Polyethylene Glycol Alkyl Ethers
Ethers of polyethylene glycol and alkyl alcohols are suitable
surfactants for use in the present invention. Examples of these
surfactants are shown here in Table 12.
Polyethylene Glycol Alkyl Ethers

| Compound | Commercial Product (Supplier) | HLB |
|---|---|---|
| PEG-2 oleyl ether, | Brij 92/93 (Atlas/ICI) | 4.9 |
| oleth-2 | | |
| PEG-3 oleyl ether, | Volpo 3 (Croda) | <10 |
| oleth-3 | | |
| PEG-5 oleyl ether, | Volpo 5 (Croda) | <10 |
| oleth-5 | | |
| PEG-10 oleyl ether, | Volpo 10 (Croda), Brij 96/97 (Atlas/ICI) | 12 |
| oleth-10 | | |
| PEG-20 oleyl ether, | Volpo 20 (Croda), Brij 98/99 (Atlas/ICI) | 15 |
| oleth-20 | | |
| PEG-4 lauryl ether, | Brij 30 (Atlas/ICI) | 9.7 |
| laureth-4 | | |
| PEG-9 lauryl ether | | >10 |
| PEG-23 lauryl ether, | Brij 35 (Atlas/ICI) | 17 |
| laureth-23 | | |
| PEG-2 cetyl ether | Brij 52 (ICI) | 5.3 |
| PEG-10 cetyl ether | Brij 56 (ICI) | 13 |
| PEG-20 cetyl ether | Brij 58 (ICI) | 16 |
| PEG-2 stearyl ether | Brij 72 (ICI) | 4.9 |
| PEG-10 stearyl ether | Brij 76 (ICI) | 12 |
| PEG-20 stearyl ether | Brij 78 (ICI) | 15 |
| PEG-100 stearyl ether | Brij 700 (ICI) | >10 |

TABLE 13
Sugar Esters
Esters of sugars are suitable surfactants for use in the present
invention. Examples of such surfactants are shown here in Table 13.
Sugar Ester Surfactants

| Compound | Commercial Product (Supplier) | HLB |
|---|---|---|
| Sucrose distearate | SUCRO ESTER 7 (Gattefosse), Crodesta | 3 |
| | F-10 (Croda) | |
| Sucrose distearate/ | SUCRO ESTER 11 (Gattefosse), | 12 |
| monostearate | Crodesta F-110 (Croda) | |
| Sucrose dipalmitate | | 7.4 |
| Sucrose monostearate | Crodesta F-160 (Croda) | 15 |
| Sucrose | SUCRO ESTER 15 (Gattefosse) | >10 |
| monopalmitate | | |
| Sucrose monolaurate | Saccharose monolaurate 1695 | 15 |
| | (Mitsubishi-Kasei) | |

TABLE 14
Polyethylene Glycol Alkyl Phenols
Several hydrophilic PEG-alkyl phenol surfactants are available,
and are suitable for use in the present invention. Examples of
these surfactants are shown here in Table 14.
Polyethylene Glycol Alkyl Phenol Surfactants

| Compound | Commercial Product (Supplier) | HLB |
|---|---|---|
| PEG-10-100 | Triton X series (Rohm & Haas), Igepal CA series | >10 |
| nonyl phenol | (GAF, USA), Antarox CA series (GAF, UK) | |
| PEG-15-100 | Triton N-series (Rohm & Haas), Igepal CO series | >10 |
| octyl phenol | (GAF, USA), Antarox CO series (GAF, UK) | |
| ether | | |

TABLE 15
Polyoxyethylene-Polyoxypropylene Block Copolymers
The POE-POP block copolymers are a unique class of polymeric
surfactants. The unique structure of the surfactants, with hydrophilic POE
and lipophilic POP moieties in well-defined ratios and positions, provides
a wide variety of surfactants suitable for use in the present invention.
These surfactants are available under various trade names, including
Synperonic PE series (ICI); Pluronic ® series (BASF), Emkalyx,
Lutrol (BASF), Supronic, Monolan, Pluracare, and Plurodac. The generic
term for these polymers is “poloxamer” (CAS 9003-11-6).
These polymers have the formula: HO(C₂H₄O)ₐ(C₃H₆O)_{b}(C₂H₄O)ₐH
where “a” and “b” denote the number of polyoxyethylene and
polyoxypropylene units, respectively.
Examples of suitable surfactants of this class are shown here in Table 15.
Since the compounds are widely available, commercial sources are not
listed in the Table. The compounds are listed by generic name, with the
corresponding “a” and “b” values.
POE-POP Block Copolymers

| | a, b values in | | |
|---|---|---|---|
| Compound | HO(C₂H₄O)ₐ(C₃H₆O)_{b}(C₂H₄O)ₐH | | HLB |
| Poloxamer 105 | a = 11 | b = 16 | 8 |
| Poloxamer 108 | a = 46 | b = 16 | >10 |
| Poloxamer 122 | a = 5 | b = 21 | 3 |
| Poloxamer 123 | a = 7 | b = 21 | 7 |
| Poloxamer 124 | a = 11 | b = 21 | >7 |
| Poloxamer 181 | a = 3 | b = 30 | |
| Poloxamer 182 | a = 8 | b = 30 | 2 |
| Poloxamer 183 | a = 10 | b = 30 | |
| Poloxamer 184 | a = 13 | b = 30 | |
| Poloxamer 185 | a = 19 | b = 30 | |
| Poloxamer 188 | a = 75 | b = 30 | 29 |
| Poloxamer 212 | a = 8 | b = 35 | |
| Poloxamer 215 | a = 24 | b = 35 | |
| Poloxamer 217 | a = 52 | b = 35 | |
| Poloxamer 231 | a = 16 | b = 39 | |
| Poloxamer 234 | a = 22 | b = 39 | |
| Poloxamer 235 | a = 27 | b = 39 | |
| Poloxamer 237 | a = 62 | b = 39 | 24 |
| Poloxamer 238 | a = 97 | b = 39 | |
| Poloxamer 282 | a = 10 | b = 47 | |
| Poloxamer 284 | a = 21 | b = 47 | |
| Poloxamer 288 | a = 122 | b = 47 | >10 |
| Poloxamer 331 | a = 7 | b = 54 | 0.5 |
| Poloxamer 333 | a = 20 | b = 54 | |
| Poloxamer 334 | a = 31 | b = 54 | |
| Poloxamer 335 | a = 38 | b = 54 | |
| Poloxamer 338 | a = 128 | b = 54 | |
| Poloxamer 401 | a = 6 | b = 67 | |
| Poloxamer 402 | a = 13 | b = 67 | |
| Poloxamer 403 | a = 21 | b = 67 | |
| Poloxamer 407 | a = 98 | b = 67 | |

TABLE 16
Sorbitan Fatty Acid Esters
Sorbitan esters of fatty acids are suitable surfactants
for use in the present invention.
Examples of these surfactants are shown here in Table 16.
Sorbitan Fatty Acid Ester Surfactants

| Compound | Commercial Product (Supplier) | HLB |
|---|---|---|
| Sorbitan monolaurate | Span-20 (Atlas/ICI), Crill 1 (Croda), | 8.6 |
| | Arlacel 20 (ICI) | |
| Sorbitan | Span-40 (Atlas/ICI), Crill 2 (Croda), | 6.7 |
| monopalmitate | Nikkol SP-10 (Nikko) | |
| Sorbitan monooleate | Span-80 (Atlas/ICI), Crill 4 (Croda), | 4.3 |
| | Crill 50 (Croda) | |
| Sorbitan monostearate | Span-60 (Atlas/ICI), Crill 3 (Croda), | 4.7 |
| | Nikkol SS-10 (Nikko) | |
| Sorbitan trioleate | Span-85 (Atlas/ICI), Crill 45 (Croda), | 4.3 |
| | Nikkol SO-30 (Nikko) | |
| Sorbitan sesquioleate | Arlacel-C (ICI), Crill 43 (Croda), | 3.7 |
| | Nikkol SO-15 (Nikko) | |
| Sorbitan tristearate | Span-65 (Atlas/ICI) Crill 35 (Croda), | 2.1 |
| | Nikkol SS-30 (Nikko) | |
| Sorbitan | Crill 6 (Croda), Nikkol SI-10 (Nikko) | 4.7 |
| monoisostearate | | |
| Sorbitan sesquistearate | Nikkol SS-15 (Nikko) | 4.2 |

TABLE 17
Lower Alcohol Fatty Acid Esters
Esters of lower alcohols (C₂ to C₄) and fatty acids (C₈ to C₁₈)
are suitable surfactants for use in the present invention.
Examples of these surfactants are shown here in Table 17.
Lower Alcohol Fatty Acid Ester Surfactants

| Compound | Commercial Product (Supplier) | HLB |
|---|---|---|
| Ethyl oleate | Crodamol EO (Croda), Nikkol EOO (Nikko) | <10 |
| Isopropyl myristate | Crodamol IPM (Croda) | <10 |
| Isopropyl palmitate | Crodamol IPP (Croda) | <10 |
| Ethyl linoleate | Nikkol VF-E (Nikko) | <10 |
| Isopropyl linoleate | Nikkol VF-IP (Nikko) | <10 |

TABLE 18
Ionic Surfactants
Ionic surfactants, including cationic, anionic and zwitterionic surfactants,
are suitable hydrophilic surfactants for use in the present invention.
Preferred anionic surfactants include fatty acid salts and bile salts.
Preferred cationic surfactants include carnitines. Specifically, preferred
ionic surfactants include sodium oleate, sodium lauryl sulfate, sodium
lauryl sarcosinate, sodium dioctyl sulfosuccinate, sodium cholate, sodium
taurocholate; lauroyl carnitine; palmitoyl carnitine; and myristoyl
carnitine. Examples of such surfactants are shown here in Table 18. For
simplicity, typical counterions are shown in the entries in the Table. It will
be appreciated by one skilled in the art, however, that any bioacceptable
counterion can be used. For example, although the fatty acids are shown
as sodium salts, other cation counterions can also be used, such as alkali
metal cations or ammonium. Unlike typical non-ionic surfactants, these
ionic surfactants are generally available as pure compounds, rather than
commercial (proprietary) mixtures. Because these compounds are readily
available from a variety of commercial suppliers, such as Aldrich, Sigma,
and the like, commercial sources are not generally listed in the Table.
Ionic Surfactants

| Compound | HLB |
|---|---|
| FATTY ACID SALTS | >10 |
| Sodium caproate | |
| Sodium caprylate | |
| Sodium caprate | |
| Sodium laurate | |
| Sodium myristate | |
| Sodium myristolate | |
| Sodium palmitate | |
| Sodium palmitoleate | |
| Sodium oleate | 18 |
| Sodium ricinoleate | |
| Sodium linoleate | |
| Sodium linolenate | |
| Sodium stearate | |
| Sodium lauryl sulfate (dodecyl) | 40 |
| Sodium tetradecyl sulfate | |
| Sodium lauryl sarcosinate | |
| Sodium dioctyl sulfosuccinate [sodium docusate (Cytec)] | |
| BILE SALTS | >10 |
| Sodium cholate | |
| Sodium taurocholate | |
| Sodium glycocholate | |
| Sodium deoxycholate | |
| Sodium taurodeoxycholate | |
| Sodium glycodeoxycholate | |
| Sodium ursodeoxycholate | |
| Sodium chenodeoxycholate | |
| Sodium taurochenodeoxycholate | |
| Sodium glycol cheno deoxycholate | |
| Sodium cholylsarcosinate | |
| Sodium N-methyl taurocholate | |
| Sodium lithocholate | |
| PHOSPHOLIPIDS | |
| Egg/Soy lecithin [Epikuron ™ (Lucas Meyer), | |
| Ovothin ™ (Lucas Meyer)] | |
| Lyso egg/soy lecithin | |
| Hydroxylated lecithin | |
| Lysophosphatidylcholine | |
| Cardiolipin | |
| Sphingomyelin | |
| Phosphatidylcholine | |
| Phosphatidyl ethanolamine | |
| Phosphatidic acid | |
| Phosphatidyl glycerol | |
| Phosphatidyl serine | |
| PHOSPHORIC ACID ESTERS | |
| Diethanolammonium polyoxyethylene-10 oleyl ether phosphate | |
| Esterification products of fatty alcohols or fatty alcohol | |
| ethoxylates with phosphoric acid or anhydride | |
| CARBOXYLATES | |
| Ether carboxylates (by oxidation of terminal OH group of | |
| fatty alcohol ethoxylates) | |
| Succinylated monoglycerides [LAMEGIN ZE (Henkel)] | |
| Sodium stearyl fumarate | |
| Stearoyl propylene glycol hydrogen succinate | |
| Mono/diacetylated tartaric acid esters of mono- and diglycerides | |
| Citric acid esters of mono-, diglycerides | |
| Glyceryl-lacto esters of fatty acids (CFR ref. 172.852) | |
| Acyl lactylates: | |
| lactylic esters of fatty acids calcium/sodium stearoyl-2-lactylate | |
| calcium/sodium stearoyl lactylate | |
| Alginate salts | |
| Propylene glycol alginate | |
| SULFATES AND SULFONATES | |
| Ethoxylated alkyl sulfates | |
| Alkyl benzene sulfones | |
| α-olefin sulfonates | |
| Acyl isethionates | |
| Acyl taurates | |
| Alkyl glyceryl ether sulfonates | |
| Octyl sulfosuccinate disodium | |
| Disodium undecylenamideo-MEA-sulfosuccinate | |
| CATIONIC Surfactants | >10 |
| Lauroyl carnitine | |
| Palmitoyl carnitine | |
| Myristoyl carnitine | |
| Hexadecyl triammonium bromide | |
| Decyl trimethyl ammonium bromide | |
| Cetyl trimethyl ammonium bromide | |
| Dodecyl ammonium chloride | |
| Alkyl benzyldimethylammonium salts | |
| Diisobutyl phenoxyethoxydimethyl benzylammonium salts | |
| Alkylpyridinium salts | |
| Betaines (trialkylglycine): | |
| Lauryl betaine (N-lauryl,N,N-dimethylglycine) | |
| Ethoxylated amines: | |
| Polyoxyethylene-15 coconut amine | |

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. In the case of inconsistencies, the present disclosure will prevail.

Where a numerical limit or range is stated, the endpoints are included. Also, all values and subranges within a numerical limit or range are specifically included as if explicitly written out.

Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A pharmaceutical composition, comprising:
at least one microparticle comprising at least one core which is at least partially coated with at least one osmotic subcoat, and at least one outer coat which at least partially coats the at least one osmotic subcoat,
wherein
said at least one core comprises at least one drug, and at least one excipient, and
said at least one osmotic subcoat comprises at least one osmotic agent and at least one osmotic deposition vehicle.

2. The composition of claim 1, wherein the at least one drug is selected from the group consisting of bupropion, carvedilol, citalopram, diltiazem, fluoxetine, metroprolol, pramipexole, quetiapine, ramipril, rivastigmine, rosiglitazone, sumatriptan, topiramate, tramadol, venlafaxine, zolpidem, memantine, dexmethylphenidate, dimebon, salts of these drugs, and combinations thereof.

3. The composition of claim 1, wherein the at least one drug is dexmethylphenidate or its salt.

4. The composition of claim 1, wherein the at least one drug is rivastigmine or its salt.

5. The composition of claim 1, wherein the at least one drug is pramipexole or its salt.

6. The composition of claim 1, wherein the at least one drug is diltiazem or its salt.

7. The composition of claim 1, wherein the at least one drug is tramadol or its salt.

8. The composition of claim 1, wherein the at least one drug is bupropion hydrochloride.

9. The composition of claim 1, wherein the at least one drug is a member of a drug class selected from the group consisting of ace-inhibitors, alkaloids, anabolic agents, analgesics, antacids, anti-allergy agents, anti-Alzheimer's Disease agents, anti-anginal drugs, antianxiety agents, anti-arrhythmia agents, antiasthmatics, antibacterial agents, anti-bipolar agents, antifungal agents, antibiotics, anticholesterolemics, anticlotting agents, anticonvulsants, anticoagulants, antidepressants, antidiarrheal preparations, anti-emetics, antihistamines, antihyperglycemic agents, antihypertensives, anti-impotence agents, anti-infectives, anti-inflammatories, antilipid agents, antimanics, anti-migraine agents, antinauseants, antineoplastics, antiobesity agents, antiparasitics, anti-Parkinsonism agents, antipsychotics, antipyretics, antispasmodics, antistroke agents, antithrombotics, antithyroid preparations, antitumor agents, antitussives, antiulcer agents, anti-uricemic agents, antiviral agents, anxiolytic agents, appetite stimulants, appetite suppressants, autoimmune disorders agents, barbiturates, beta-blocking agents, blood glucose-lowering agents, bronchodilators, cardiovascular agents, cerebral dilators, chelating agents, cholecystekinin antagonists, chemotherapeutic agents, cholesterol-reducing agents, cognition activators, cognitive enhancers, contraceptives, coronary dilators, cough suppressants, decongestants, deodorants, dermatological agents, diabetes agents, diuretics, emollients, enzymes, erythropoietic drugs, expectorants, fertility agents, fungicides, gastrointestinal agents, growth regulators, anti-headache agents, anti-cluster headache agents, hormone replacement agents, hyperglycemic agents, hypnotic agents, hypoglycemic agents, ion-exchange resins, laxatives, migraine treatments, mineral supplements, mucolytics, narcotics, neuroleptics, neuromuscular drugs, non-steroidal anti-inflammatories (NSAIDs), nutritional additives, peripheral vasodilators, polypeptides, prostaglandins, psychotropics, renin inhibitors, respiratory stimulants, anti-restless leg syndrome agents, sedatives, steroids, stimulants, sympatholytics, thyroid preparations, tranquilizers, uterine relaxants, vaginal preparations, vasoconstrictors, vasodilators, vertigo agents, vitamins, wound healing agents, and combinations thereof.

10. The composition of claim 9, comprising at least one drug selected from the group consisting of anti-Alzheimer's Disease agents.

11. The composition of claim 9, comprising at least one drug selected from the group consisting of anti-arrhythmia agents.

12. The composition of claim 9, comprising at least one drug selected from the group consisting of anti-Parkinsonism agents.

13. The composition of claim 9, comprising at least one drug selected from the group consisting of anti-restless leg syndrome agents.

14. The composition of claim 9, comprising at least one drug selected from the group consisting of anti-cluster headache agents.

15. The composition of claim 9, comprising at least one drug selected from the group consisting of anti-bipolar agents.

16. The composition of claim 9, comprising at least one drug selected from the group consisting of antihypertensives.

17. The composition of claim 9, comprising at least one drug selected from the group consisting of analgesics.

18. The composition of claim 1, wherein the at least one drug is selected from the group consisting of acetazolamide, acetaminophen, acetic acid, acetohexamide, acetylsalicylic acid, buffered acetylsalicylic acid; acrivastine, acyclovir, albuterol, albuterol sulfate, alcohol, alfaxalone, alkaline phosphatase, allantoin, aloe, alprostadil, aluminum acetate, aluminum carbonate, aluminum chlorohydrate, aluminum hydroxide, alprozolam, amino acids, aminobenzoic acid, amlodipine besylate, amoxicillin, ampicillin, amsacrine, amsalog, anethole, apomorphine, ascorbic acid, aspartame, aspirin, astemizole, atenolol, atorvastatin calcium, azatidine, azatidine maleate, azithromycin, bacitracin, balsam peru, BCNU (carmustine), becampicillin hydrochloride, beclomethasone diproprionate, benzalkonium chloride, benzocaine, benzoic acid, benzophenones, benzoyl peroxide, benzquinamide, benzquinamide hydrochloride, betamethasone, bethanechol, biotin, bisacodyl, bismuth subsalicylate, bomyl acetate, bromopheniramine, bromopheniramine maleate, bupropion hydrochloride, buspirone, caffeine, calamine, calcium carbonate, calcium casinate, calcium hydroxide, camphor, captopril, carbenicillin indanyl sodium, carvedilol, cascara sagrada, castor oil, cefaclor, cefadroxil, celicoxib, cephalexin, centrizine, centrizine hydrochloride, cetirizine, cetyl alcohol, cetylpyridinium chloride, chelated minerals, chlorambucil, chloramphenicol, chlorcyclizine hydrochloride, chlordiazepoxide, chlorhexidine gluconate, chloroxylenol, chloropentostatin, chlorpheniramine, chlorpheniramine maleate, chlorpheniramine tannate, chlorpromazine, chlorpropamide, chlorthalidone, chlorzolamide, cholestyramine resin, choline bitartrate, chondrogenic stimulating protein, cimetidine, cimetidine hydrochloride, cinnamedrine hydrochloride, cinnarizine, cisapride, citalopram, citric acid, clarithromycin, clemastine, clemastine flumarate, clonidine, clonidine hydrochloride, clorfibrate, cocoa butter, cod liver oil, codeine, codeine fumarate, codeine phosphate, cortisone acetate; cotrimoxazole, ciprofloxacin HCl, cyanocobalamin, cyclizine hydrochloride, cyproheptadine, cyproheptadine hydrochloride, dexmethylphenidate, danthron, dexbromopheniramine maleate, dextromethorphan, dextromethorphan hydrohalide, diazepam, dibucaine, dichloralphenazone, diclofen, alkali metal salts of diclofen, diclofenac sodium, dicumarol, digitoxin, digoxin, dihydroergotamine, hydrogenates of dihydroergotamine, mesylates of dihydroergotamine, diltiazem, dimebon, dimenhydrinate, dimethicone, dioxybenzone, diphenhydramine, diphenhydramine citrate, diphenhydramine hydrochloride, divalproex, alkali metal salts of divalproex, docusate calcium, docusate potassium, docusate sodium, donepezil, doxazosin, doxepin, doxepin hydrochloride, doxycycline hydrate, doxylamine succinate, dronabinol, echinomycin, econazole, efaroxan, enalapril, enalaprilic acid, enoxacin, ephedrine, epinephrine bitartrate, ergotamine, ergotamine tartrate, erythromycin, erythropoietin, estropipate, ethinyl estradiol, etomidate, eucalyptol, famotidine, fenoprofen, metal salts of fenoprofen, ferrous fumarate, ferrous gluconate, ferrous sulfate, fluconazole, fluoxetine, fluoxymesterone, folic acid, fosphenytoin, 5-fluorouracil (5-FU), fluoxetine, fluoxetine hydrochloride, flurbiprofen, fluspirilene, furosemide, gabapentan, gentamicin, gemfibrozil, glipizide, glycerine, glyceryl stearate, granisetron, granisetron hydrochloride, griseofulvin, guafenesin, hexylresorcinol, hydrochlorothiazide, hydrocodone, tartrates of hydrocodone, hydrocortisone, hydrocortisone acetate, 8-hydroxyquinoline sulfate, hydroxyzine, hydroxyzine pamoate, hydrochloride salts of hydroxyzine, ibuprofen, indomethacin, inositol, insulin, iodine, ipecac, iron, iroxicam, isosorbide, monoand dinitrates of isosorbide, isoxicam, kaolin, ketamine, ketanserin, ketoprofen, lactic acid, lanolin, L-DOPA, lecithin, leuprolide acetate, levocabastine, lidocaine, lidocaine hydrochloride, lifinopril, liotrix, lisinopril, lomustine, loperamide, loratadine, lovastatin, magnesium carbonate, magnesium hydroxide, magnesium salicylate, magnesium trisilicate, meclizine, meclizine hydrochloride, mefenamic acid, meclofenamic acid, meclofenamate sodium, medroxyprogesterone acetate, meloxicam, memantine, methenamine mandelate, menthol, meperidine hydrochloride, metaproterenol sulfate, methanstenolone, methscopolamine, nitrates of methscopolamine, methsergide, methsergide maleate, methyl nicotinate, methyl salicylate, methyl cellulose, methsuximide, 17-methyltestosterone, metoclopramide, halides of metoclopramide, hydrates of metoclopramide, metronidazole, metronidazole hydrochloride, metoprolol, metoprotol tartrate, mianserin, miconazole nitrate, mineral oil, minocycline, minoxidil, mioflazine, morphine, nadolol, naproxen, sodium salts of naproxen, alkali metal salts of naproxen, nifedipine, neomycin sulfate, niacin, niacinamide, nicotine, nicotinamide; nimesulide; nitroglycerine; nonoxynol-9; norethindrone and its acetate; nystatin, octoxynol, octoxynol-9, octyl dimethyl PABA, octyl methoxycinnamate, omega-3 polyunsaturated fatty acids, omeprazole, ondansetron, ondansetron hydrochloride, oxfendazole, oxolinic acid, oxybenzone, oxtriphylline, para-aminobenzoic acid (PABA), padimate-O, paramethadione, paroxetine, penfluridole, penicillin G, pentastatin, peppermint oil, pentaerythritol tetranitrate, pentobarbital sodium, perphenazine, phenelzine sulfate, phenindamine, phenindamine tartrate, pheniramine maleate, phenobarbital, phenol, phenolphthalein, phenylephrine, tannates of phenylephrine, hydrochlorides of phenylephrine, phenylpropanolamine, phenylpropanolamine hydrochloride, phenyloin, pirmenol, piroxicam, salts of piroxicam, polymicin B sulfate, potassium chloride, potassium nitrate, pramipexole, pramiracetin, pramoxine, pramoxine hydrochloride, prazepam, prazosin, prednisolone, procainamide hydrochloride, procaterol, promethazine, promethazine hydrochloride, propoxyphene, propoxyphene hydrochloride, napsylate, prochlorperazine, prochlorperazine maleate, propanolol, propanolol hydrochloride, promethazine, promethazine hydrochloride, propanolol, prostacyclin, pseudoephedrine, sulfates of pseudoephedrine, hydrochlorides of pseudoephedrine, pyridoxine, pyrolamine, hydrochlorides of pyrolamine, tannates of pyrolamine, quetiapine, quinapril, quinidine gluconate, quinidine sulfate, quinestrol, ralitoline, ramipril, ranitidine, resorcinol, retinol, riboflavin, rivastigmine, rosiglitazone, salicylic acid, scopolamine, sertraline, sesame oil, shark liver oil, sildenafil citrate, simethicone, sodium bicarbonate, sodium citrate, sodium fluoride, sodium monofluorophosphate, spiramycin, spironolactone, sucralfate, sulfanethoxazole, sulfasalazine, sulfur, sulpiride, sumatriptan, sumatriptan succinate, tacrine, tacrine hydrochloride, terconazole, terfenadine, testosterone, tetracycline, tetracycline hydrochloride, tetrahydroaminoacridine, theophylline, thiabendazole, thiethylperazine, thiethylperazine maleate, thioperidone, thiothixene hydrochloride, timolol, timolol maleate, tolmetin, tolnaftate, topiramate, tramadol, tretinoin, triazolam, trimetrexate, trimazosin, triclosan, trimethobenzamide, trimethobenzamide hydrochloride, tripelennamine, tripelennamine hydrochloride, tripolidine hydrochloride, troleandomycin, tubulazole, undecylenic acid, valdecoxib, vancomycin, venlafaxine, verapamil HCl, vidaribine phosphate, virazole, vitamin A, vitamin C, vitamin D, vitamin B1, vitamin B2, vitamin B3, vitamin B4, vitamin B5, vitamin B6, vitamin B7, vitamin B9, vitamin B12, vitamin E, vitamin K, witch hazel, xylometazoline hydrochloride, zinc, zinc sulfate, zinc undecylenate, ziprasidone, zolpidem, salts thereof, and combinations thereof.

19. The composition of claim 1, wherein the at least one drug is in a salt form.

20. The composition of claim 1, wherein the at least one drug is an enantiomer.

21. The composition of claim 1, wherein the at least one drug is a mixture of enantiomers.

22. The composition of claim 1, wherein the at least one drug is a diastereomer.

23. The composition of claim 1, wherein the at least one drug is a prodrug.

24. The composition of claim 1, wherein the shape of the at least one microparticle is spherical.

25. The composition of claim 1, wherein the at least one microparticle is pearl shaped.

26. The composition of claim 1, wherein, in the at least one core, the at least one drug is enveloped by the at least one excipient.

27. The composition of claim 1, wherein, in the at least one core, the at least one drug is dispersed in a matrix with the at least one excipient.

28. The composition of claim 27, wherein the matrix is an immediate release matrix.

29. The composition of claim 27, wherein the matrix is a modified release matrix.

30. The composition of claim 1, wherein the at least one osmotic subcoat fully surrounds the at least one core.

31. The composition of claim 1, wherein the at least one outer coat fully surrounds the at least one osmotic subcoat.

32. The composition of claim 1, wherein the at least one osmotic subcoat fully surrounds the at least one core, and wherein the at least one outer coat fully surrounds the at least one osmotic subcoat.

33. The composition of claim 1, wherein the at least one drug has a solubility such that ≦about 10 parts water will dissolve one part of the drug, so long as the total amount of water to dissolve the at least one drug is not 0 parts.

34. The composition of claim 1, wherein the at least one drug has a solubility such that ≧30 parts of water will dissolve one part of the drug.

35. The composition of claim 1, wherein the at least one drug has a solubility such that from more than about 10 parts water to less than about 30 parts water will dissolve one part of the drug.

36. The composition of claim 1, wherein the total amount of the at least one drug present in the composition ranges from about 200 mg to about 10,000 mg.

37. The composition of claim 1, wherein the total amount of the at least one drug present in the composition ranges from more than about 20 mg to less than about 200 mg.

38. The composition of claim 1, wherein the total amount of the at least one drug present in the composition ranges from less than about 20 mg to greater than 0 mg.

39. The composition of claim 1, wherein the at least one microparticle has a diameter ranging from about 50 μm to about 800 μm.

40. The composition of claim 1, wherein the at least one drug is present in a positive amount of ≦20 mg, wherein at least 30 parts of water is used to dissolve 1 part of the drug, and wherein ≧90% of the drug is released from the composition within 24 hours of placing the composition into an external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

41. The composition of claim 1, wherein the drug is present in a positive amount of ≦20 mg, and wherein ≧90% of the drug is released from the composition within 24 hours of placing the composition into an external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

42. The composition of claim 41,
wherein the at least one drug is pramipexole,
wherein the volume of the dissolution medium is 500 ml,
wherein the dissolution medium is stirred at 50 rotations per minute, and
wherein the dissolution medium is the phosphate buffer having a pH of 6.8.

43. The composition of claim 41,
wherein the at least one drug is rivastigmine,
wherein the volume of the dissolution medium is 500 ml,
wherein the dissolution medium is stirred at 50 rotations per minute, and
wherein the dissolution medium is the 0.1N HCl aqueous solution.

44. The composition of claim 41,
wherein the at least one drug is rivastigmine,
wherein the volume of the dissolution medium is 500 ml,
wherein the dissolution medium is stirred at 50 rotations per minute, and
wherein the dissolution medium is the acetate buffer having a pH of 4.5.

45. The composition of claim 41,
wherein the at least one drug is rivastigmine,
wherein the volume of the dissolution medium is 500 ml,
wherein the dissolution medium is stirred at 50 rotations per minute, and
wherein the dissolution medium is the phosphate buffer having a pH of 6.8.

46. The composition of claim 1,
wherein the at least one drug is present in a positive amount of ≦20 mg,
wherein there is increased release of the at least one drug from the composition within 24 hours of placing the composition into a first external environment of use compared to an otherwise identical or similar second composition comprising the at least one drug but not containing an osmotic subcoat placed into a second external environment of use,
wherein the first external environment of use and the second external environment of use are identical or similar, and
wherein each external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

47. The composition of claim 1, wherein at least thirty parts of water is used to dissolve one part of the drug, and wherein ≧90% of the drug is released from the composition within 24 hours of placing the composition into an external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

48. The composition of claim 1, wherein at least thirty parts of water is required to dissolve one part of the drug, wherein there is increased release of the at least one drug from the composition within 24 hours of placing the composition into a first external environment of use compared to an otherwise identical or similar composition comprising the at least one drug but not containing an osmotic subcoat placed into a second external environment of use,
wherein the first external environment of use and the second external environment of use are identical or similar, and,
wherein each external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

49. The composition of claim 1, wherein the at least one core comprises from about 0.1% by weight to about 99.9% by weight of the at least one excipient and from about 0.1% by weight to about 99.9% by weight of the at least one drug.

50. The composition of claim 1, wherein the at least one osmotic subcoat comprises from about 1% by weight to about 99% by weight of the at least one osmotic deposition vehicle and from about 1% by weight to about 99% by weight of the at least one osmotic agent.

51. The composition of claim 1, wherein the weight of the least one outer coat is equal to from about 1% to about 99% of the weight of the at least one core.

52. The composition of claim 49, wherein the total weight of the at least one core is the weight of the at least one drug plus the weight of the at least one excipient.

53. The composition of claim 50, wherein the total weight of the at least one osmotic subcoat is the weight of the at least one osmotic agent plus the weight of the at least one osmotic deposition vehicle.

54. The composition of claim 1, wherein the at least one core comprises 97.5% by weight of the at least one excipient and 2.5% by weight of the at least one drug, and wherein the total % by weight of the at least one drug and the at least one excipient is 100% of the weight total of the at least one core.

55. The composition of claim 54, wherein the at least one drug is pramipexole and wherein the at least one excipient is glyceryl monostearate.

56. The composition of claim 1, wherein the at least one core comprises 40% by weight of the at least one excipient and 60% by weight of the at least one drug and wherein the total % by weight of the at least one drug and the at least one excipient is 100% of the weight total of the at least one core.

57. The composition of claim 56, wherein the at least one drug is diltiazem and wherein the at least one excipient is glyceryl monostearate.

58. The composition of claim 1, wherein the at least one core comprises 90% by weight of the at least one excipient and 10% by weight of the at least one drug and wherein the total % by weight of the at least one drug and the at least one excipient is 100% of the weight total of the at least one core.

59. The composition of claim 58, wherein the at least one drug is rivastigmine and the at least one excipient is glyceryl monostearate.

60. The composition of claim 1, wherein the at least one excipient is selected from the group consisting of a spheronization aid, a solubility enhancer, a disintegrating agent, a diluent, a lubricant, a binder, a filler, a suspending agent, an emulsifying agent, an anti-foaming agent, a falvouring agent, a colouring agent, a chemical stabilizer, a pH modifier, a swelling agent, and mixtures thereof.

61. The composition of claim 60, wherein the at least one excipient comprises a spheronization aid, and wherein the spheronization aid is selected from the group consisting of a distilled monoglyceride, a hydrogenated oil, a fatty acid salt, a polyol, a polyoxyethylene ether, an esterified polyoxyethylene, a wax, a wax like material, a thermo-plastic polymer, a thermo-softening polymer, and combinations thereof.

62. The composition of claim 61, wherein the spheronization aid is selected from the group consisting of glyceryl monostearate, glyceryl behenate, glyceryl dibehenate, glyceryl palmitostearate, hydrogenated castor oil, magnesium stearate, calcium stearate, mannitol, sorbitol, xylitol, stearic acid, palmitic acid, sodium lauryl sulfate, PEG-32 distearate, PEG-150 distearate, cetostearyl alcohol, carnauba wax, white wax, paraffin wax, povidone, a cellulose ether, a polyvinylalcohol, and combinations thereof.

63. The composition of claim 62, wherein the spheronization aid comprises glyceryl monostearate.

64. The composition of claim 62, wherein the spheronization aid comprises glyceryl behenate.

65. The composition of claim 62, wherein the spheronization aid comprises glyceryl palmitostearate.

66. The composition of claim 60, wherein the at least one excipient comprises a filler, and wherein the filler is selected from the group consisting of calcium phosphate dibasic, tricalcium phosphate, calcium carbonate, a starch, a modified starch, microcrystalline cellulose, sucrose, dextrose, a maltodextrin, lactose, fructose, and combinations thereof.

67. The composition of claim 66, wherein the filler comprises microcrystalline cellulose.

68. The composition of claim 66, wherein the filler comprises lactose.

69. The composition of claim 60, wherein the at least one excipient comprises a solubility enhancer, and wherein the solubility enhancer is selected from the group consisting of a macrogol fatty acid ester, a polyethylene glycol, a polyethylene-polypropylene glycol, sorbitol, a propylene glycol, a pentaerythritol, and mixtures thereof.

70. The composition of claim 69, wherein the at least one excipient comprises at least one polyethylene-polypropylene glycol, and wherein the average molecular weight of the polyethylene-propylene glycol ranges from 9,480-14,600.

71. The composition of claim 69, wherein the at least one excipient comprises at least one polyethylene glycol, and wherein the average molecular weight of the polyethylene glycol ranges from 3,000-4,800.

72. The composition of claim 69, wherein the at least one excipient comprises at least one macrogel fatty acid ester, wherein the melting point of the macrogel fatty acid ester ranges from 40° C. to 60° C., and wherein the hydrophilic-lipophilic balance value of the macrogel fatty acid ester is 13.

73. The composition of claim 60, wherein the at least one excipient comprises a binder, and wherein the binder is selected from the group consisting of polyethylene glycol, stearic acid, a low melting point wax, and mixtures thereof.

74. The composition of claim 1, wherein the at least one osmotic agent is selected from the group consisting of sodium chloride, sodium bromide, sodium bisulfate, potassium acid tartrate, citric acid, sodium citrate, fumaric acid, mannitol, sucrose, and mixtures thereof.

75. The composition of claim 74, wherein the at least one osmotic agent comprises sodium chloride.

76. The composition of claim 74, wherein the at least one osmotic agent comprises sodium citrate.

77. The composition of claim 74, wherein the at least one osmotic agent comprises fumaric acid.

78. The composition of claim 74, wherein the at least one osmotic agent comprises mannitol.

79. The composition of claim 1, wherein said at least one osmotic deposition vehicle is selected from the group consisting of a polyvinyl pyrrolidone, a hydroxyethyl cellulose, a hydroxypropyl cellulose, a low molecular weight hydroxypropyl methylcellulose (HPMC), a polymethacrylate, an ethyl cellulose and mixtures thereof.

80. The composition of claim 79, wherein the at least one osmotic deposition vehicle comprises a low molecular weight hydroxypropyl methylcellulose (HPMC).

81. The composition of claim 79, wherein the at least one osmotic deposition vehicle comprises hypromellose substitution type 2910 with a nominal viscosity of 6 cP.

82. The composition of claim 79, wherein the at least one osmotic deposition vehicle comprises hypromellose substitution type 2906 with a nominal viscosity of 3 cP.

83. The composition of claim 79, wherein the at least one osmotic deposition vehicle comprises polyvinylpyrrolidone with a molecular weight of 30,000.

84. The composition of claim 1, wherein said at least one microparticle is partially coated with the at least one outer coat.

85. The composition of claim 1, wherein the at least one core is partially coated with the at least one osmotic subcoat.

86. The composition of claim 1, wherein the at least one outer coat comprises at least one polymer, polymeric material, or polymeric dispersion.

87. The composition of claim 86, wherein the at least one polymer, polymeric material, or polymeric dispersion is selected from the group consisting of a poly (meth)acrylate neutral copolymer aqueous dispersion, a (meth)acrylate neutral copolymer, a polyvinyl acetate aqueous dispersion, a polyvinyl acetate, an ethylcellulose, an ethylcellulose aqueous dispersion, a dispersion of a poly(ethyl acrylate and methyl acrylate), a poly(ethyl acrylate and methyl acrylate) and combinations thereof.

88. The composition of claim 87, wherein the at least one polymer, polymeric material, or polymeric dispersion comprises a poly(meth)acrylate neutral copolymer.

89. The composition of claim 87, wherein the at least one polymer, polymeric material, or polymeric dispersion comprises a polyvinyl acetate.

90. The composition of claim 87, wherein the at least one polymer, polymeric material, or polymeric dispersion comprises an ethylcellulose.

91. The composition of claim 87, wherein the at least one polymer, polymeric material, or polymeric dispersion comprises a poly(ethyl acrylate and methyl acrylate).

92. The composition of claim 1, wherein the at least one outer coat comprises at least one glidant.

93. The composition of claim 92, wherein the at least one glidant is selected from the group consisting of talc, magnesium stearate, calcium silicate, glyceryl monostearate, and combinations thereof.

94. The composition of claim 93, wherein the at least one glidant comprises talc.

95. The composition of claim 93, wherein the at least one glidant comprises glyceryl monostearate.

96. The composition of claim 93, wherein the at least one glidant comprises calcium silicate.

97. The composition of claim 93, wherein the at least one glidant comprises glyceryl monostearate.

98. The composition of claim 1, wherein the at least one outer coat comprises at least one emulsifier.

99. The composition of claim 98, wherein the at least one emulsifier comprises polyoxethylene sorbitan monooleate.

100. The composition of claim 1, wherein the outer coat comprises at least one anti-foaming agent.

101. The composition of claim 100, wherein the at least one anti-foaming agent comprises simethicone.

102. The composition of claim 1, wherein the outer coat comprises at least one plasticizer.

103. The composition of claim 102, wherein the plasticizer is selected from the group consisting of acetyltributyl citrate, triacetin, dibutyl sebacate, and mixtures thereof.

104. The composition of claim 103, wherein the plasticizer comprises acetyltributyl citrate.

105. The composition of claim 103, wherein the plasticizer comprises triacetin.

106. The composition of claim 103, wherein the plasticizer comprises dibutyl sebacate.

107. The composition of claim 1, wherein the outer coat does not comprise a pore former.

108. The composition of claim 1, wherein the at least one drug is present in a positive amount of ≦20 mg, wherein ≧90% of the drug is released from the composition within 24 hours of placing the composition into an external environment of use, wherein the at least one outer coat does not comprise a pore former,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

109. The composition of claim 1, wherein the at least one outer coat does not comprise a pore former, wherein the at least one drug is present in a positive amount of ≦20 mg, wherein there is increased release of the at least one drug from the composition within 24 hours of placing the composition into a first external environment of use compared to an otherwise identical or similar second composition comprising the at least one drug but not containing an osmotic subcoat placed into a second external environment of use,
wherein the first external environment of use and the second external environment of use are identical or similar, and
wherein each external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

110. The composition of claim 1, wherein at least 30 parts of water is required to dissolve one part of the drug, wherein ≧90% of the drug is released from the composition within 24 hours of placing the composition into an external environment of use, wherein the at least one outer coat does not comprise a pore former, and
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

111. The composition of claim 1, wherein at least 30 parts of water is required to dissolve one part of the drug, wherein the at least one outer coat does not comprise a pore former, wherein there is increased release of the at least one drug from the composition within 24 hours of placing the composition into a first external environment of use compared to an otherwise identical or similar second composition comprising the at least one drug but not containing an osmotic subcoat placed into a second external environment of use,
wherein the first external environment of use and the second external environment of use are identical or similar, and
wherein each external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

112. The composition of claim 1, wherein the at least one outer coat comprises a pore former.

113. The composition of claim 112, wherein the pore former comprises at least one material selected from the group consisting of hypromellose substitution type 2910 having a viscosity ranging from 5 cP to 7 cP, hypromellose substitution type 2906 having a viscosity ranging from 2 cP to 4 cP, a polyvinylpyrrolidione having a molecular weight ranging from 20,000 to 40,000, and mixtures thereof.

114. The composition of claim 1, wherein the at least one outer coat comprises at least one drug, and wherein the at least one drug of the at least one outer coat may be the same or different from the drug in the core.

115. The composition of claim 1, wherein the composition is an extended release composition.

116. The composition of claim 1, wherein the composition is a delayed release composition.

117. The composition of claim 116, wherein the composition, when placed into an external environment of use, provides a drug release profile wherein a predetermined lag time is substantially independent of the pH of the external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

118. The composition of claim 1, wherein the composition is a sustained release composition.

119. The composition of claim 1, wherein the composition, when placed into an external environment of use, provides a drug release profile that does not include a lag time,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

120. The composition of claim 1, wherein the at least one outer coat does not comprise a seal coat.

121. The composition of claim 1, wherein the at least one osmotic agent and the at least one osmotic deposition vehicle are present in the at least one osmotic subcoat in amounts sufficient to achieve an osmotic pressure gradient across the at least one outer coat for the transport of a solvent or aqueous fluid from an external environment of use into said core, and transport of said drug from said core to said external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

122. The composition of claim 121, wherein the release of the drug from the core to the external environment of use is effected by both the osmotic pressure gradient and passive diffusion.

123. The composition of claim 1, wherein the at least one osmotic deposition vehicle does not effect the rate and/or extent of release of the at least one drug.

124. A method of administering at least one drug, the method comprising:
administering to a subject in need thereof a pharmaceutical composition, comprising:
at least one microparticle comprising at least one core which is at least partially coated with at least one osmotic subcoat, and at least one outer coat which at least partially coats the at least one osmotic subcoat,
wherein
said at least one core comprises at least one drug, and at least one excipient, and
said at least one osmotic subcoat comprises at least one osmotic agent and at least one osmotic deposition vehicle.

125. The method of claim 124, wherein the composition is in the form of a tablet.

126. The method of claim 124, wherein the composition is in the form of a capsule.

127. The method of claim 124, wherein the composition is administered with a dose sipping technology.

128. The method of claim 124, wherein the composition is in the form of an orally disintegrating tablet.

129. A method of treating a disease in a subject in need thereof, said method comprising:
administering to a subject in need thereof an effective amount of a pharmaceutical composition, said pharmaceutical composition comprising:
at least one microparticle comprising at least one core which is at least partially coated with at least one osmotic subcoat, and at least one outer coat which at least partially coats the at least one osmotic subcoat,
wherein
said at least one core comprises at least one drug, and at least one excipient, and
said at least one osmotic subcoat comprises at least one osmotic agent and at least one osmotic deposition vehicle
said at least one drug is effective for treating said disease in the subject in need thereof.

130. A method of making a pharmaceutical composition, said pharmaceutical composition comprising:
at least one microparticle comprising at least one core which is at least partially coated with at least one osmotic subcoat, and at least one outer coat which at least partially coats the at least one osmotic subcoat,
wherein
said at least one core comprises at least one drug, and at least one excipient, and
said at least one osmotic subcoat comprises at least one osmotic agent and at least one osmotic deposition vehicle,
wherein said method comprises
(1) forming the at least one core which comprises said at least one drug and at least said one excipient;
(2) coating, at least partially, said at least one core with said at least one osmotic subcoat, and
(3) coating, at least partially, said at least one osmotic subcoat with said at least one outer coat.

131. The method of claim 130, wherein the at least one osmotic subcoat fully surrounds the at least one core, and wherein the at least one outer coat fully surrounds the at least one osmotic subcoat.

132. The method of claim 130, wherein said at least one outer coat does not contain a pore former.

133. A pharmaceutical composition, which is prepared by a method comprising:
(1) forming at least one core which comprises at least one drug and at least one excipient;
(2) coating, at least partially, said at least one core with at least one osmotic subcoat, and
(3) coating, at least partially, said at least one osmotic subcoat with at least one outer coat.

134. The composition of claim 133, wherein the at least one osmotic subcoat fully surrounds the at least one core, and wherein the at least one outer coat fully surrounds the at least one osmotic subcoat.

135. The composition of claim 133, wherein the at least one outer coat does not comprise a pore former.

136. A method of controlling the rate and/or extent of release of at least one drug from the core of a microparticle comprising at least one core into an external environment of use which is at least partially coated with at least one osmotic subcoat, and at least one outer coat at least partially coats the at least one osmotic subcoat, wherein
said at least one core comprises at least one drug and at least one excipient,
said at least one osmotic subcoat comprises at least one osmotic agent and at least one osmotic deposition vehicle,
the method comprising
controlling the thickness of the at least one outer coat, thereby controlling the release rate of the at least one drug from the core of the microparticle into the external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

137. The method of claim 136, wherein the at least one outer coat comprises a stable monolithic coating comprising an aqueous dispersion of a neutral ester copolymer without any functional groups, a poly glycol having a melting point greater than 55° C., and at least one pharmaceutically acceptable excipient.

138. The method of claim 136, wherein the at least one outer coat comprises a water insoluble, water permeable polymer, a water soluble polymer, and a plasticizer.

139. The method of claim 136, wherein the at least one osmotic subcoat fully surrounds the at least one core, and wherein the at least one outer coat fully surrounds the at least one osmotic subcoat.

140. The method of claim 136, wherein the at least one outer coat does not comprise a pore former.

141. A pharmaceutical composition, comprising
at least one core which is at least partially coated with at least one osmotic subcoat,
at least one outer coat which at least partially coats the at least one osmotic subcoat,
wherein the at least one core comprises at least one drug and at least one excipient,
wherein the at least one osmotic subcoat comprises at least one osmotic agent and at least one osmotic deposition vehicle, and
a means for releasing the at least one drug from the composition.

142. The composition of claim 141, wherein the at least one osmotic subcoat fully surrounds the at least one core, and wherein the at least one outer coat fully surrounds the at least one osmotic subcoat.

143. The composition of claim 141, wherein the at least one outer coat does not comprise a pore former.

144. A method for controlling the rate and/or extent of release of at least one drug from a composition, into an external environment of use, the composition comprising
at least one microparticle comprising at least one core which is at least partially coated with at least one osmotic subcoat, and at least one outer coat which at least partially coats the at least one osmotic subcoat,
wherein
said at least one core comprises at least one drug, and at least one excipient, and
said at least one osmotic subcoat comprises at least one osmotic agent and at least one osmotic deposition vehicle,
wherein the method comprises at least one of the following:
controlling the amount of the at least one osmotic agent in the at least one osmotic subcoat, and
controlling the thickness of the at least one outer coat, thereby controlling the release rate of the at least one drug from the core of the microparticle into the external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

145. The method of claim 144, wherein the at least one outer coat comprises a plasticizer, and wherein the method further comprises:
controlling the amount of the plasticizer in the at least one outer coat.

146. The composition of claim 1, wherein the drug is present in an amount of more than about 200 mg, and wherein ≧90% of the drug is released from the composition within 24 hours of placing the composition into an external environment of use,
wherein the external environment of use is a dissolution medium,
wherein the temperature of the dissolution medium is 37° C.+/−0.5° C.,
wherein the volume of the dissolution medium is selected from the group consisting of 500 ml and 900 ml,
wherein the dissolution medium is selected from the group consisting of water, a 0.1N HCl aqueous solution, a 0.1N HCl aqueous solution with sodium chloride added in an amount of 15.75 g/litre of the solution, a 0.1N HCl aqueous solution with added 0.1 wt % Cetrimide wherein the wt % is based on the weight of the solution, USP Buffer having a pH of 1.5, an acetate buffer having a pH of 4.5, a phosphate buffer having a pH of 6.5, a phosphate buffer having a pH of 6.8, a phosphate buffer having a of pH 7.4, and a 0.1N HCl aqueous solution with sodium chloride added in an amount of 14 g/litre of the solution,
wherein the dissolution medium is stirred by a USP type II paddle at 50 rotations per minute or 100 rotations per minute, and
wherein the pressure of the atmosphere on the dissolution medium is 1 atmosphere.

147. The composition of claim 146,
wherein the at least one drug is diltiazem,
wherein the volume of the dissolution medium is 900 ml,
wherein the dissolution medium is stirred at 100 rotations per minute, and
wherein the dissolution medium is water.
